(19) United States
(12) Patent Application Publication
(10) Pub. No.: US 2024/0218081 A1
(43) Pub. Date: Jul. 4, 2024

(54) METHODS FOR THE ADMINISTRATION OF ADAMTS BINDING IMMUNOGLOBULINS

(71) Applicants: NOVARTIS AG, Basel (CH); Merck Patent GmbH, Darmstadt (DE)

(72) Inventors: Gérard FLESCH, Wittenheim (FR); Hans GUEHRING, Geisenheim (DE); Matthias Klaus SCHIEKER, Muenchen (DE)

(21) Appl. No.: 18/557,721

(22) PCT Filed: Apr. 29, 2022
(86) PCT No.: PCT/IB2022/054001

§ 371 Date: Oct. 27, 2023

Related U.S. Application Data
(60) Provisional application No. 63/362,372, filed on Apr. 1, 2022.
(60) Provisional application No. 63/182,561, filed on Apr. 30, 2021.

(51) Int. Cl. C07K 16/40; A61K 39/00

(52) U.S. Cl. CPC C07K 16/40; A61K 2039/505; A61K 2039/545; C07K 2317/31; C07K 2317/565

(57) ABSTRACT

Disclosed herein are methods and dosage regimens for the treatment of osteoarthritis (e.g., knee osteoarthritis). The methods and dosage regimens comprise administration of an ADAMTS5 inhibiting polypeptide in a therapeutically effective amount.

### CROSS-REFERENCE TO RELATED APPLICATIONS

This application claims priority to U.S. Provisional Application Ser. No. 63/362,372, filed Apr. 1, 2022, and U.S. Provisional Application Ser. No. 63/182,561, filed Apr. 30, 2021, each of which is incorporated by reference herein in its entirety for all purposes.

### SEQUENCE LISTING

The instant application contains a Sequence Listing which has been submitted electronically in ASCII format and is hereby incorporated by reference in its entirety. Said ASCII copy, created on Apr. 20, 2022, is named PAT059112-WO-PCT_SL.txt and is about 171,234 bytes in size.

### TECHNICAL FIELD

The present disclosure provides methods of treating diseases or disorders in which ADAMTS activity is involved by administering therapeutically effective amounts of an ADAMTS inhibiting polypeptide.

### BACKGROUND

Osteoarthritis (OA), the most common joint disorder in the world, is a serious, chronic progressive joint disease with no known cure that has been associated with an increased risk of premature mortality. The lifetime risk of developing symptomatic knee and hip OA is 45% and 25% respectively; it accounts for over 90% of knee and hip arthroplasties. The number of people affected globally by OA rose 48% from 1990 to 2019, and it is predicted that the burden of knee OA will continue to escalate in tandem with the rising prevalence of longevity and obesity.

According to the Osteoarthritis Research Society International (OARSI), the disease manifests first as a molecular derangement (abnormal joint tissue metabolism) followed by anatomic, and/or physiologic derangements (characterized by cartilage degradation, bone remodeling, osteophyte formation, joint inflammation and loss of normal joint function). Clinically, OA is associated with joint pain, swelling and stiffness that can lead to activity limitations, sleep interruption, fatigue, depression, anxiety, and ultimately loss of independence and reduced quality of life.

According to clinical practice guidelines, nonsurgical treatment of OA comprises both pharmacologic and nonpharmacologic modalities (such as patient education, referral to a physiotherapist, exercise, weight reduction, walking aids, knee braces, and footwear). All presently available pharmacologic therapies for OA provide symptomatic relief by transiently reducing pain but have not been shown to delay structural progression of OA. In addition, long-term use of these therapies is associated with serious adverse effects, including fall-related bone fractures, substance dependence and/or abuse in patients receiving opioids, cardiovascular risk, and upper gastrointestinal bleeding in OA patients receiving nonsteroidal anti-inflammatory drugs.

Total knee replacement surgery (TKR) is considered when adequate attempts of symptomatic pharmacologic therapies fail. However, not all patients are satisfied with the result, or benefit from joint replacement surgery. In a long-term outcome study in an OA cohort with a high prevalence of multiple affected joints and comorbidities, only half of those who received joint replacement achieved a good surgical outcome, defined as improved pain and reduced disability. As longevity increases and OA prevalence rises (even at younger ages), the ever increasing number of joint replacement surgeries causes a growing public health burden.

Overall, the burden of OA is considerable, and includes the growing costs for pharmacological and surgical treatments, the increasing expenses for individuals and their families who must adapt their lives and homes to the disease, and the impact on society due to lost work productivity. There is unmet a significant need for a disease-modifying osteoarthritis drug (DMOAD) that can slow or halt OA disease progression by inhibiting structural deterioration and improve symptoms. Although many putative agents have been investigated, no pharmaceutical agent has been approved for clinical use as a DMOAD.

Extensive loss of aggrecan, driven mainly by the action of aggrecanases, is detectable in different joint diseases and is a hallmark of OA. A Disintegrin and Metalloproteinase with Thrombospondin motifs 5 (ADAMTS5, also known as ADAMTS11, ADMP-2, and Aggrecanase-2) is a major aggrecanase present in cartilage, and various lines of evidence indicate that ADAMTS5 is a principal enzyme involved in the pathogenesis of osteoarthritis. For example, ADAMTS5 KO mice are protected against cartilage degeneration. In human cartilage explants and chondrocytes, knockdown of ADAMTS5 attenuated aggrecan breakdown, suggesting that this enzyme also may be involved in human tissues. ADAMTS5 generated aggrecan fragments are also detected in the synovial fluid and serum of OA patients. Therefore, inhibition of aggrecan fragment generation by ADAMTS5 has the potential to halt cartilage degeneration, as well as to reduce pain.

### BRIEF SUMMARY

The present disclosure relates to methods for treating osteoarthritis (e.g., knee osteoarthritis) by administering therapeutically effective amounts of an ADAMTS5 inhibiting polypeptide to a human subject. In some embodiments, the ADAMTS5 inhibiting polypeptide comprises at least one immunoglobulin single variable domain (ISVD) comprising three complementarity determining regions (CDRs), wherein the complementary determining regions are CDR1 to CDR3, in which (i) CDR1 is selected from the group consisting of SEQ ID NOs: 21, 35, 20, 22, 25, 33, 28, 24, 23, 26, 27, 29, 30, 31, 32 and 34; (ii) CDR2 is selected from the group consisting of SEQ ID NOs: 37, 53, 36, 40, 50, 51, 44, 45, 43, 39, 38, 41, 119, 42, 46, 47, 48, 49 and 52; and (iii) CDR3 is selected from the group consisting of SEQ ID NOs: 55, 118, 71, 54, 58, 68, 69, 62, 63, 61, 57, 56, 59, 60, 64, 65, 66, 67 and 70. In some embodiments, the ADAMTS5 inhibiting polypeptide comprises at least one ISVD comprising 3 CDRs, wherein the CDRs are CDR1 to CDR3, in which:
CDR1 is SEQ ID NO: 21, CDR2 is SEQ ID NO: 37 and CDR3 is SEQ ID NO: 55;
CDR1 is SEQ ID NO: 35, CDR2 is SEQ ID NO: 53 and CDR3 is SEQ ID NO: 118;
CDR1 is SEQ ID NO: 35, CDR2 is SEQ ID NO: 53 and CDR3 is SEQ ID NO: 71;
CDR1 is SEQ ID NO: 20, CDR2 is SEQ ID NO: 36 and CDR3 is SEQ ID NO: 54;
CDR1 is SEQ ID NO: 22, CDR2 is SEQ ID NO: 36 and CDR3 is SEQ ID NO: 54;
CDR1 is SEQ ID NO: 25, CDR2 is SEQ ID NO: 40 and CDR3 is SEQ ID NO: 58;
CDR1 is SEQ ID NO: 33, CDR2 is SEQ ID NO: 50 and CDR3 is SEQ ID NO: 68;
CDR1 is SEQ ID NO: 33, CDR2 is SEQ ID NO: 51 and CDR3 is SEQ ID NO: 69;
CDR1 is SEQ ID NO: 28, CDR2 is SEQ ID NO: 44 and CDR3 is SEQ ID NO: 62;
CDR1 is SEQ ID NO: 28, CDR2 is SEQ ID NO: 45 and CDR3 is SEQ ID NO: 63;
CDR1 is SEQ ID NO: 28, CDR2 is SEQ ID NO: 43 and CDR3 is SEQ ID NO: 61;
CDR1 is SEQ ID NO: 24, CDR2 is SEQ ID NO: 39 and CDR3 is SEQ ID NO: 57;
CDR1 is SEQ ID NO: 23, CDR2 is SEQ ID NO: 38 and CDR3 is SEQ ID NO: 56;
CDR1 is SEQ ID NO: 26, CDR2 is SEQ ID NO: 41 and CDR3 is SEQ ID NO: 59;
CDR1 is SEQ ID NO: 27, CDR2 is SEQ ID NO: 119 and CDR3 is SEQ ID NO: 60;
CDR1 is SEQ ID NO: 27, CDR2 is SEQ ID NO: 42 and CDR3 is SEQ ID NO: 60;
CDR1 is SEQ ID NO: 29, CDR2 is SEQ ID NO: 46 and CDR3 is SEQ ID NO: 64;
CDR1 is SEQ ID NO: 30, CDR2 is SEQ ID NO: 47 and CDR3 is SEQ ID NO: 65;
CDR1 is SEQ ID NO: 31, CDR2 is SEQ ID NO: 48 and CDR3 is SEQ ID NO: 66;
CDR1 is SEQ ID NO: 32, CDR2 is SEQ ID NO: 49 and CDR3 is SEQ ID NO: 67; or
CDR1 is SEQ ID NO: 34, CDR2 is SEQ ID NO: 52 and CDR3 is SEQ ID NO: 70.

In some embodiments, the CDR 1 is SEQ ID NO: 21, CDR2 is SEQ ID NO: 37, and CDR3 is SEQ ID NO: 55. In some embodiments, the ADAMTS5 inhibiting polypeptide comprises at least one ISVD comprising 3 CDRs, wherein the CDRs are CDR1 to CDR3, in which CDR1 is SEQ ID NO. In some embodiments, the ISVD is chosen from the group consisting of SEQ ID NOs: 2, 116, 19, 1, 3, 6, 16, 17, 10, 11, 9, 5, 4, 7, 8, 117, 12, 13, 14, 15, 18, and amino acids 1-124 of SEQ ID NO: 129. In some embodiments, the ISVD is amino acids 1-124 of SEQ ID NO: 129.

In some embodiments, the ADAMTS5 inhibiting polypeptide comprises two or more ISVDS, wherein a) at least a first ISVD specifically binds a first antigenic determinant, epitope, part, domain, subunit or conformation of ADAMTS5; and wherein, b) at least a second ISVD specifically binds a second antigenic determinant, epitope, part, domain, subunit or conformation of serum albumin. In some embodiments, the first ISVD is chosen from the group consisting of SEQ ID NOs: 2, 116, 19, 1, 3, 6, 16, 17, 10, 11, 9, 5, 4, 7, 8, 117, 12, 13, 14, 15, 18, and amino acids 1-124 of SEQ ID NO: 129. In some embodiments, the second ISVD specifically binding serum albumin is chosen from the group consisting of SEQ ID NOs: 131, 132, 133, 134, 135, 136, 137, 138, 139, 140, 141, 142, 143, 144, or 145. In some embodiments, the first ISVD is amino acids 1-124 of SEQ ID NO: 129 and the second ISVD is SEQ ID NO: 138. In some embodiments, the ADAMTS5 inhibiting polypeptide is selected from the group consisting of SEQ ID NO: 129 (clone 577 2F3^{so}-Alb), SEQ ID NO: 130 (clone 579 2F3^{so}-093-Alb), SEQ ID NO: 120 (clone 4 2A12-Alb), SEQ ID NO: 121 (clone 5 2D7-Alb), SEQ ID NO: 122 (clone 6 2F3-Alb), SEQ ID NO: 123 (clone 69 049-Alb), SEQ ID NO: 124 (clone 70 9D3-Alb), SEQ ID NO: 125 (clone 71 3B2-Alb), SEQ ID NO: 126 (clone 129 2F3-093-Alb), SEQ ID NO: 127 (clone 130 049-093-Alb), and SEQ ID NO: 128 (clone 131 9D3-093-Alb). In some embodiments, the ADAMTS5 inhibiting polypeptide is SEQ ID NO: 129 (clone 577 2F3^{so}-Alb).

In some embodiments, the present disclosure provides a method of treating osteoarthritis comprising administering at least one dose of 75-300 mg of an ADAMTS5 inhibiting polypeptide to a human subject. In some embodiments, the osteoarthritis is knee osteoarthritis. In some embodiments, the dose is 75 mg, 150 mg, or 300 mg. In some embodiments, the ADAMTS5 inhibiting polypeptide is administered once a month. In some embodiments, administration of the ADAMTS5 inhibiting polypeptide decreases the formation of ARGS fragments. In some embodiments, administration of the ADAMTS5 inhibiting polypeptide decreases pain in the osteoarthritis affected joint as determined by KOOS score and/or WOMAC score. In some embodiments, administration of the ADAMTS5 inhibiting polypeptide maintains the structure of the osteoarthritis affected joint as determined by automated segmentation in MRI.

In some embodiments, the present disclosure provides use of an ADAMTS5 inhibiting polypeptide in the treatment of osteoarthritis. In some embodiments, the treatment comprises administering at least one dose of 75-300 mg of an ADAMTS5 inhibiting polypeptide to a human subject. In some embodiments, the osteoarthritis is knee osteoarthritis. In some embodiments, the dose is 75 mg, 150 mg, or 300 mg. In some embodiments, the ADAMTS5 inhibiting polypeptide is administered once a month. In some embodiments, administration of the ADAMTS5 inhibiting polypeptide decreases the formation of ARGS fragments. In some embodiments, administration of the ADAMTS5 inhibiting polypeptide decreases pain in the osteoarthritis affected joint as determined by KOOS score and/or WOMAC score. In some embodiments, administration of the ADAMTS5 inhibiting polypeptide maintains the structure of the osteoarthritis affected joint as determined by automated segmentation in MRI.

In some embodiments, the present disclosure provides an ADAMTS5 inhibiting polypeptide for use in the treatment of osteoarthritis, e.g., the treatment comprising administering at least one dose of 75-300 mg of a ADAMTS5 inhibiting polypeptide to a human subject. In some embodiments, the osteoarthritis is knee osteoarthritis. In some embodiments, the dose is 75 mg, 150 mg, or 300 mg. In some embodiments, the ADAMTS5 inhibiting polypeptide is administered once a month. In some embodiments, administration of the ADAMTS5 inhibiting polypeptide decreases the formation of ARGS fragments. In some embodiments, administration of the ADAMTS5 inhibiting polypeptide decreases pain in the osteoarthritis affected joint as determined by KOOS score and/or WOMAC score. In some embodiments, administration of the in ADAMTS5 inhibiting polypeptide maintains the structure of the osteoarthritis affected joint as determined by automated segmentation in MRI.

In some embodiments, the present disclosure provides an ADAMTS5 inhibiting polypeptide for use in the manufacture of a medicament for the treatment of osteoarthritis. In some embodiments, the use comprises administering at least one dose of 75-300 mg of a ADAMTS5 inhibiting polypeptide to a human subject. In some embodiments, the osteoarthritis is knee osteoarthritis. In some embodiments, the dose is 75 mg, 150 mg, or 300 mg. In some embodiments, the ADAMTS5 inhibiting polypeptide is administered once a month. In some embodiments, administration of the ADAMTS5 inhibiting polypeptide decreases the formation of ARGS fragments. In some embodiments, administration of the ADAMTS5 inhibiting polypeptide decreases pain in the osteoarthritis affected joint as determined by KOOS score and/or WOMAC score. In some embodiments, administration of the ADAMTS5 inhibiting polypeptide maintains the structure of the osteoarthritis affected joint as determined by automated segmentation in MRI.

In some embodiments, the present disclosure provides a pharmaceutical composition comprising an ADAMTS5 inhibiting polypeptide for use in the treatment of osteoarthritis. In some embodiments, the treatment comprises administering at least one dose of 75-300 mg of a ADAMTS5 inhibiting polypeptide to a human subject. In some embodiments, the osteoarthritis is knee osteoarthritis. In some embodiments, the dose is 75 mg, 150 mg, or 300 mg. In some embodiments, the ADAMTS5 inhibiting polypeptide is administered once a month. In some embodiments, administration of the ADAMTS5 inhibiting polypeptide decreases the formation of ARGS fragments. In some embodiments, administration of the ADAMTS5 inhibiting polypeptide decreases pain in the osteoarthritis affected joint as determined by KOOS score and/or WOMAC score. In some embodiments, administration of the ADAMTS5 inhibiting polypeptide maintains the structure of the osteoarthritis affected joint as determined by automated segmentation in MRI.

### DESCRIPTION OF THE DRAWINGS

FIG. 1 depicts the subject disposition for a single dose escalation study.

FIG. 2 depicts the subject disposition for a multiple ascending dose study.

FIG. 3 depicts the serum concentrations of Compound 1 after single dose on linear and semi-logarithmic scale (Arithmetic mean±SD), X-axis scaled to 120 hours (5 days).

FIG. 4 depicts the serum concentrations of Compound 1 after single dose on linear and semi-logarithmic scale (Arithmetic mean±SD), X-axis scaled to 1776 hours (74 days).

FIG. 5 depicts the serum concentration-time profiles of Compound 1 for all dose levels and regimens (weekly and every other week) on linear and semi-logarithmic scales (day 1).

FIG. 6 depicts the serum concentration-time profiles of Compound 1 for all dose levels and regimens (weekly and every other week) on linear and semi-logarithmic scales (all study days).

FIG. 7 depicts percent change from baseline in serum ARGS after single dose of Compound 1 in healthy male participants. ARGS=aggrecan degradation neoepitope.

FIG. 8 depicts maximum inhibitory effect of Compound 1 in healthy male participants measured as percent decrease in ARGS. ARGS=aggrecan degradation neoepitope. IQR=inter-quartile range. Emin=maximum inhibitory effect (Imax); Bottom and top edges of the box indicate 25th and 75th percentiles, respectively; solid line inside the box indicates median; and diamond indicates mean value. Black circles indicate the range of values are outside of IQR but within 1.5*IQR.

FIG. 9 depicts the median (Q1 to Q3) percent change from baseline (%) versus time profiles for all dose levels on linear scale. ARGS=aggrecan degradation neoepitope. Q1S=weekly; Q2S=every other week.

### DETAILED DESCRIPTION

Provided herein are methods for treating and/or preventing diseases or disorders in an individual in which the activity of an ADAMTS enzyme is involved. In some embodiments, the ADAMTS enzyme is ADAMTS5. The method comprises administering a polypeptide that inhibits the activity of ADAMTS to a subject in an amount effective to treat or prevent a symptom of the disease or disorder. Examples of diseases or disorders that may be treated with an ADAMTS inhibiting polypeptide include arthropathies and chondrodystrophies, arthritic disease (such as osteoarthritis, rheumatoid arthritis, gouty arthritis, psoriatic arthritis), traumatic rupture or detachment, achondroplasia, costochondritis, Spondyloepimetaphyseal dysplasia, spinal disc herniation, lumbar disk degeneration disease, degenerative joint disease, relapsing polychondritis, osteochondritis dissecans and aggrecanopathies. In some embodiments, the disease or disorder is an arthritic disease. In some embodiments, the disease or disorder is osteoarthritis.

ADAMTS5 inhibiting polypeptides have previously been described in WO 2018/220234 (WO '234), the contents of which are fully incorporated by reference. The WO '234 publication contemplates administration of ADAMTS5 inhibiting polypeptides as a potential treatment for ADAMTS5 associated diseases or disorders, but does not provide any specific guidance on dosing regimens or dosage amounts. Instead, WO '234 offers a wide range of potential dosages and leaves selection of a particular dosing regimen to a clinician. Thus, there remains a need to establish therapeutically effective dosage amounts and dosing regimens for the administration of ADAMTS5 inhibiting polypeptides. The present inventors have now met that need with the doses and dosing regimens described herein.

### Definitions

The term “subject” refers to an animal, human or non-human, to whom treatment according to the methods of the present disclosure is provided. Veterinary and non-veterinary applications are contemplated. The term includes, but is not limited to, mammals, e.g., humans, other primates, pigs, rodents such as mice and rats, rabbits, guinea pigs, hamsters, cows, horses, cats, dogs, sheep and goats. Typical subjects include humans, farm animals, and domestic pets such as cats and dogs.

The term “treatment,” “treating,” or “treat” is herein defined as therapeutic measures for the reduction or amelioration of the progression, severity and/or duration of an undesired physiological change or disorder (e.g., an ADAMTS5 related disorder, such as an arthritic disease (e.g., OA)), or the amelioration of one or more symptoms (e.g., one or more discernible symptoms) of the disorder resulting from the administration of one or more therapeutic agents. In some embodiments the terms “treatment,” “treating,” or “treat” refer to the reduction or stabilization of the progression of a disorder, such as OA, either physically by, e.g., reduction or stabilization of a discernible symptom, physiologically by, e.g., reduction or stabilization of a physical parameter, or both. For purpose of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and reversal (whether partial or total), whether detectable or undetectable.

A subject is “in need of” a treatment if such subject would benefit biologically, medically, or in quality of life from such treatment.

The term “pharmaceutically acceptable” means a nontoxic material that does not interfere with the effectiveness of the biological activity of the active ingredient(s).

The term “administration” or “administering” of the subject compound means providing a drug, a modified derivative of a drug, or a prodrug to a subject in need of treatment.

The term “dosing regimen,” as used herein, refers to the treatment plan specifically indicating the administration pattern of a drug over a period of time. The dosing regimen defines the amount of a drug and the number and frequency of its administrations that is employed in the treatment of a disease.

The term “dose” refers to a specified amount of a drug administered at one time. As used herein, the dose is the amount of the drug that elicits a therapeutic effect.

The term “therapeutically effective amount” refers to an amount of a drug that will elicit the desired biological or medical response in a subject, for example, at least partially ameliorate symptoms, alleviate conditions, slow or delay progression, or reverse a disorder or disease.

The term “ADAMTS5 inhibiting polypeptide” refers to a polypeptide that inhibits an activity of ADAMTS5, such as a protease activity. For example, the protease activity may be cleavage of aggrecan, versican, brevican, neurocan, decorin, and/or biglycan. In some embodiments, the ADAMTS5 inhibiting polypeptide antagonizes aggrecanase activity of ADAMTS5.

The terms ARGS, ARGS fragment, and ARGS neo-epitope refer to the aggrecan degradation neoepitope. In some embodiments, an ARGS fragment or neo-epitope may comprise the sequence alanine-arginine-glycine-serine (SEQ ID NO: 175).

For the purposes of comparing two or more amino acid sequences, the percentage of “sequence identity” between a first amino acid sequence and a second amino acid sequence (also referred to herein as “amino acid identity”) may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e., as an “amino acid difference” as defined herein. Alternatively, the degree of sequence identity between two amino acid sequences may be calculated using any known computer algorithm for determining the degree of sequence identity for amino acid sequences.

When comparing two amino acid sequences, the term “amino acid(s) difference” refers to an insertion, deletion or substitution of a single amino acid residue on a position of the first sequence, compared to the second sequence; it being understood that two amino acid sequences can contain one, two, or more such amino acid differences. The amino acid(s) difference can be any one, two, three, four or maximal five substitutions, deletions or insertions, or any combination thereof, that either improve the properties of the ADAMTS inhibiting polypeptide or that at least do not detract too much from the desired properties.

Sequences are disclosed in the main body of the description and in a separate sequence listing according to WIPO standard ST.25. A SEQ ID specified with a specific number should be the same in the main body of the description and in the separate sequence listing. By way of example SEQ ID NO: 1 should define the same sequence in both, the main body of the description and in the separate sequence listing. Should there be a discrepancy between a sequence definition in the main body of the description and the separate sequence listing (if e.g. SEQ ID NO: 1 in the main body of the description erroneously corresponds to SEQ ID NO: 2 in the separate sequence listing) then a reference to a specific sequence in the application, in particular of specific embodiments, is to be understood as a reference to the sequence in the main body of the application and not to the separate sequence listing. In other words, a discrepancy between a sequence definition/designation in the main body of the description and the separate sequence listing is to be resolved by correcting the separate sequence listing to the sequences and their designation disclosed in the main body of the application which includes the description, examples, figures and claims.

### ADAMTS5 Binding Polypeptides

The present disclosure relates to the administration of a polypeptide comprising at least one immunoglobulin single variable domain (ISVD) binding ADAMTS5, wherein the ISVD binding ADAMTS5 does not bind ADAMTS4, MMP1, or MMP14. In some embodiments, the polypeptide specifically binds ADAMTS5 from Homo sapiens (SEQ ID NO: 149), Mus musculus (SEQ ID NO: 153), Cavia porcellus (SEQ ID NO: 152), Bos taurus (SEQ ID NO: 150), Macaca mulatta (SEQ ID NO: 154) and/or Rattus norvegicus (SEQ ID NO: 151). In some embodiments, the ADTAMS5 binding polypeptide binds Homo sapiens ADAMTS5 (SEQ ID NO: 149) (see Table 1). In some embodiments, the present disclosure relates to the administration of an ADAMTS5 inhibiting polypeptide.

TABLE 1
Miscellaneous Amino acid sequences: Name and short description (“ID”),
SEQ ID NO:s (“SEQ”) and amino acid sequences (“sequences”)

| species | SEQ ID | Amino acid sequence |
|---|---|---|
| human | 149 | MLLGWASLLLCAFRLPLAAVGPAATPAQDKAGQPPTAAAAAQPRRRQGEEVQE |
| ADAM- | | RAEPPGHPHPLAQRRRSKGLVQNIDQLYSGGGKVGYLVYAGGRRFLLDLERDGS |
| TS5 | | VGIAGFVPAGGGTSAPWRHRSHCFYRGTVDGSPRSLAVFDLCGGLDGFFAVKHA |
| Q9UNA0-1 | | RYTLKPLLRGPWAEEEKGRVYGDGSARILHVYTREGFSFEALPPRASCETPASTPE |
| | | AHEHAPAHSNPSGRAALASQLLDQSALSPAGGSGPQTWWRRRRRSISRARQVEL |
| | | LLVADASMARLYGRGLQHYLLTLASIANRLYSHASIENHIRLAVVKVVVLGDKD |
| | | KSLEVSKNAATTLKNFCKWQHQHNQLGDDHEEHYDAAILFTREDLCGHHSCDT |
| | | LGMADVGTICSPERSCAVIEDDGLHAAFTVAHEIGHLLGLSHDDSKFCEETFGSTE |
| | | DKRLMSSILTSIDASKPWSKCTSATITEFLDDGHGNCLLDLPRKQILGPEELPGQT |
| | | YDATQQCNLTFGPEYSVCPGMDVCARLWCAVVRQGQMVCLTKKLPAVEGTPC |
| | | GKGRICLQGKCVDKTKKKYYSTSSHGNWGSWGSWGQCSRSCGGGVQFAYRHC |
| | | NNPAPRNNGRYCTGKRAIYRSCSLMPCPPNGKSFRHEQCEAKNGYQSDAKGVKT |
| | | FVEWVPKYAGVLPADVCKLTCRAKGTGYYVVFSPKVTDGTECRLYSNSVCVRG |
| | | KCVRTGCDGIIGSKLQYDKCGVCGGDNSSCTKIVGTFNKKSKGYTDVVRIPEGAT |
| | | HIKVRQFKAKDQTRFTAYLALKKKNGEYLINGKYMISTSETIIDINGTVMNYSGW |
| | | SHRDDFLHGMGYSATKEILIVQILATDPTKPLDVRYSFFVPKKSTPKVNSVTSHGS |
| | | NKVGSHTSQPQWVTGPWLACSRTCDTGWHTRTVQCQDGNRKLAKGCPLSQRPS |
| | | AFKQCLLKKC |
| bovine | 150 | MLLGWAALMLCALRLPPVAAGPTAAPAQDKAGQPRAAAVAAAAQPRGRRGEE |
| (AA | | AQEPAEPPGHPHPLAPQRGSRGLVQNIDQLYSGGGKVGYLVYAGGRRFLLDLER |
| residues | | DDSVGAAGLVPAGGGPNATRRHRGHCFYRGTVDGSPRSLAVFDLCGGLDGFFA |
| 1-626) | | VKRARYTLQPLLRGPWAEAEGDARVYGDESARILHVYTREGFSFEALPPRTSCET |
| | | HASPPGARERPPAPSRPDGRWALAPQQLPGQSAPSSDGSQGPRTWWRRRRRSISR |
| | | ARQVELLLVADASMARMYGRGLQHYLLTLASIANKLYSHASIENHIRLVVVKVV |
| | | VLGDKDKSLEVSKNAATTLKNFCKWQHQHNQLGDDHEEHYDAAILFTREDLCG |
| | | HHSCDTLGMADVGTICSPERSCAVIEDDGLHAAFTVAHEIGHLLGLSHDDSKFCE |
| | | ENFGSTEDKRLMSSILTSIDASKPWSKCTSATITEFLDDGHGNCLLDLPRKQIPGPE |
| | | ELPGQTYDASQQCNLTFGPEYSVCPGMDVCARLWCAVVRQGQMVCLTKKLPA |
| | | VEGTPCGKGRICLQGKCVDKTKKKYYSTSSHGNWGSWGSWGQCSRSCGGGVQF |
| | | AYRHCNNPAPRNNGRYCTGKRAIYRSCSVTPCPHHHHHHHHHH |
| rat | 151 | MRLEWASLLLLLLLLCASCLALAADNPAAAPAQDKTRQPRAAAAAAQPDQRQW |
| (AA | | EETQERGHPQPLARQRRSSGLVQNIDQLYSGGGKVGYLVYAGGRRFLLDLERDD |
| residues | | TVGAAGGIVTAGGLSASSGHRGHCFYRGTVDGSPRSLAVFDLCGGLDGFFAVKH |
| 1-619) | | ARYTLKPLLRGSWAESERVYGDGSSRILHVYTREGFSFEALPPRTSCETPASPSGA |
| | | QESPSVHSSSRRRTELAPQLLDHSAFSPAGNAGPQTWWRRRRRSISRARQVELLL |
| | | VADSSMAKMYGRGLQHYLLTLASIANRLYSHASIENHIRLAVVKVVVLTDKSLE |
| | | VSKNAATTLKNFCKWQHQHNQLGDDHEEHYDAAILFTREDLCGHHSCDTLGMA |
| | | DVGTICSPERSCAVIEDDGLHAAFTVAHEIGHLLGLSHDDSKFCEENFGSTEDKRL |
| | | MSSILTSIDASKPWSKCTSATITEFLDDGHGNCLLDVPRKQILGPEELPGQTYDAT |
| | | QQCNLTFGPEYSVCPGMDVCARLWCAVVRQGQMVCLTKKLPAVEGTPCGKGRI |
| | | CLQGKCVDKTKKKYYSTSSHGNWGSWGPWGQCSRSCGGGVQFAYRHCNNPAP |
| | | RNSGRYCTGKRAIYRSCSVIPCPHHHHHHHHHH |
| guinea pig | 152 | MLLGWASLLLCAFRLPQAAASAAAAPAQDKAGQPRAAAAAPQPRRRQGEHAPL |
| (AA | | RVEPPGHPHALAPQRRGRGLLQSIDRLYSGGGKVGYLVYAGGRRFLLDLERDGS |
| residues | | VGAAGLFPAGGGLSAPRRHRSHCFYRGTVDGSPRSLAVFDLCGGLRGFFAVKHA |
| 1-622) | | RYTVKPLLRGPWAEADTPRVYGDESARIPHVYTREGFSFEALPPRASCETPASQP |
| | | GPHERPPAHNSPGRHSTVDPQLPELSALSPAGDPGQQIWWRRRRRSISRARQVEL |
| | | LLVADGSMAKMYGRGLQHYLLTLASIANRLYSHASIENHIRLAVVKVVVLGDKD |
| | | KSLEVSKNAATTLKNFCKWQHQHNQLGDDHEEHYDAAILFTREDLCGHHSCDT |
| | | LGMADVGTICSPERSCAVIEDDGLHAAFTVAHEIGHLLGLSHDDSKFCEENFGLT |
| | | EDKRLMSSILTSIDASKPWSKCTSATMTEFLDDGHGNCLLDVPRKQIPSPEELPGQ |
| | | TYDATQQCNLTFGPEYSVCPGMDVCARLWCAVVRQGQMVCLTKKLPAVEGTP |
| | | CGKGRICLQGKCVDKTKKKYYSTSSHGNWGSWGPWGQCSRSCGGGVQFAYRH |
| | | CNNPAPRNSGRYCTGKRAIYRSCSVTPCPHHHHHHHHHH |
| mouse | 153 | MRLEWAPLLLLLLLLSASCLSLAADSPAAAPAQDKTRQPQAAAAAAEPDQPQGE |
| (AA | | ETRERGHLQPLAGQRRSGGLVQNIDQLYSGGGKVGYLVYAGGRRFLLDLERDDT |
| residues | | VGAAGSIVTAGGGLSASSGHRGHCFYRGTVDGSPRSLAVFDLCGGLDGFFAVKH |
| 1-622) | | ARYTLKPLLRGSWAEYERIYGDGSSRILHVYNREGFSFEALPPRASCETPASPSGP |
| | | QESPSVHSRSRRRSALAPQLLDHSAFSPSGNAGPQTWWRRRRRSISRARQVELLL |
| | | VADSSMARMYGRGLQHYLLTLASIANRLYSHASIENHIRLAVVKVVVLTDKDTS |
| | | LEVSKNAATTLKNFCKWQHQHNQLGDDHEEHYDAAILFTREDLCGHHSCDTLG |
| | | MADVGTICSPERSCAVIEDDGLHAAFTVAHEIGHLLGLSHDDSKFCEENFGTTED |
| | | KRLMSSILTSIDASKPWSKCTSATITEFLDDGHGNCLLDLPRKQILGPEELPGQTY |
| | | DATQQCNLTFGPEYSVCPGMDVCARLWCAVVRQGQMVCLTKKLPAVEGTPCG |
| | | KGRVCLQGKCVDKTKKKYYSTSSHGNWGSWGPWGQCSRSCGGGVQFAYRHCN |
| | | NPAPRNSGRYCTGKRAIYRSCSVTPCPHHHHHHHHHH |
| cynomolgus | 154 | MLLGWASLLLCAFRLPLAAAGPAAAPAQDKAGQPATAAAAAQPRRRQGEEVQE |
| monkey | | RTEPPGHPHPLAQRRSSKGLVQNIDQLYSGGGKVGYLVYAGGRRFLLDLERDGS |
| (AA | | VGTAGFVPTEGGTSAPWRHRSHCFYRGTVDGSPRSLAVFDLCGGLDGFFAVKHA |
| residues | | RYTLKPLLRGPWAEEETRRVYGDGSARILHVYTREGFSFEALQPRASCETPASTPE |
| 1-622) | | PHERPPAHSNPGGRAALASQLLDQSAVSPAGGPGPQTWWRRRRRSISRARQVEL |
| | | LLVADASMARLYGRGLQHYLLTLASIANRLYSHASIENHIRLAVVKVVVLGDKD |
| | | KSLEVSKNAATTLKNFCKWQHQHNQLGDDHEEHYDAAILFTREDLCGHHSCDT |
| | | LGMADVGTICSPERSCAVIEDDGLHAAFTVAHEIGHLLGLSHDDSKFCEETFGSTE |
| | | DKRLMSSILTSIDASKPWSKCTSATITEFLDDGHGNCLLDQPRKQILGPEELPGQT |
| | | YDATQQCNLTFGPEYSVCPGMDVCARLWCAVVRQGQMVCLTKKLPAVEGTPC |
| | | GKGRICLQGKCVDKTKKKYYSTSSHGNWGSWGSWGQCSRSCGGGVQFAYRHC |
| | | NNPAPRNNGRYCTGKRAIYRSCGLMPCPHHHHHHHHHH |
| human | 155 | MTTLLWVFVTLRVITAAVTVETSDHDNSLSVSIPQPSPLRVLLGTSLTIPCYFIDPM |
| aggrecan | | HPVTTAPSTAPLAPRIKWSRVSKEKEVVLLVATEGRVRVNSAYQDKVSLPNYPAI |
| | | PSDATLEVQSLRSNDSGVYRCEVMHGIEDSEATLEVVVKGIVFHYRAISTRYTLD |
| | | FDRAQRACLQNSAIIATPEQLQAAYEDGFHQCDAGWLADQTVRYPIHTPREGCY |
| | | GDKDEFPGVRTYGIRDTNETYDVYCFAEEMEGEVFYATSPEKFTFQEAANECRR |
| | | LGARLATTGHVYLAWQAGMDMCSAGWLADRSVRYPISKARPNCGGNLLGVRT |
| | | VYVHANQTGYPDPSSRYDAICYTGEDFVDIPENFFGVGGEEDITVQTVTWPDMEL |
| | | PLPRNITEGEARGSVILTVKPIFEVSPSPLEPEEPFTFAPEIGATAFAEVENETGEAT |
| | | RPWGFPTPGLGPATAFTSEDLVVQVTAVPGQPHLPGGVVFHYRPGPTRYSLTFEE |
| | | AQQACPGTGAVIASPEQLQAAYEAGYEQCDAGWLRDQTVRYPIVSPRTPCVGDK |
| | | DSSPGVRTYGVRPSTETYDVYCFVDRLEGEVFFATRLEQFTFQEALEFCESHNAT |
| | | ATTGQLYAAWSRGLDKCYAGWLADGSLRYPIVTPRPACGGDKPGVRTVYLYPN |
| | | QTGLPDPLSRHHAFCFRGISAVPSPGEEEGGTPTSPSGVEEWIVTQVVPGVAAVPV |
| | | EEETTAVPSGETTAILEFTTEPENQTEWEPAYTPVGTSPLPGILPTWPPTGAETEES |
| | | TEGPSATEVPSASEEPSPSEVPFPSEEPSPSEEPFPSVRPFPSVELFPSEEPFPSKEPSP |
| | | SEEPSASEEPYTPSPPEPSWTELPSSGEESGAPDVSGDFTGSGDVSGHLDFSGQLSG |
| | | DRASGLPSGDLDSSGLTSTVGSGLTVESGLPSGDEERIEWPSTPTVGELPSGAEILE |
| | | GSASGVGDLSGLPSGEVLETSASGVGDLSGLPSGEVLETTAPGVEDISGLPSGEVL |
| | | ETTAPGVEDISGLPSGEVLETTAPGVEDISGLPSGEVLETTAPGVEDISGLPSGEVL |
| | | ETTAPGVEDISGLPSGEVLETAAPGVEDISGLPSGEVLETAAPGVEDISGLPSGEVL |
| | | ETAAPGVEDISGLPSGEVLETAAPGVEDISGLPSGEVLETAAPGVEDISGLPSGEVL |
| | | ETAAPGVEDISGLPSGEVLETAAPGVEDISGLPSGEVLETAAPGVEDISGLPSGEVL |
| | | ETAAPGVEDISGLPSGEVLETAAPGVEDISGLPSGEVLETAAPGVEDISGLPSGEVL |
| | | ETTAPGVEEISGLPSGEVLETTAPGVDEISGLPSGEVLETTAPGVEEISGLPSGEVLE |
| | | TSTSAVGDLSGLPSGGEVLEISVSGVEDISGLPSGEVVETSASGIEDVSELPSGEGL |
| | | ETSASGVEDLSRLPSGEEVLEISASGFGDLSGVPSGGEGLETSASEVGTDLSGLPSG |
| | | REGLETSASGAEDLSGLPSGKEDLVGSASGDLDLGKLPSGTLGSGQAPETSGLPS |
| | | GFSGEYSGVDLGSGPPSGLPDFSGLPSGFPTVSLVDSTLVEVVTASTASELEGRGTI |
| | | GISGAGEISGLPSSELDISGRASGLPSGTELSGQASGSPDVSGEIPGLFGVSGQPSGF |
| | | PDTSGETSGVTELSGLSSGQPGVSGEASGVLYGTSQPFGITDLSGETSGVPDLSGQ |
| | | PSGLPGFSGATSGVPDLVSGTTSGSGESSGITFVDTSLVEVAPTTFKEEEGLGSVEL |
| | | SGLPSGEADLSGKSGMVDVSGQFSGTVDSSGFTSQTPEFSGLPSGIAEVSGESSRA |
| | | EIGSSLPSGAYYGSGTPSSFPTVSLVDRTLVESVTQAPTAQEAGEGPSGILELSGAH |
| | | SGAPDMSGEHSGFLDLSGLQSGLIEPSGEPPGTPYFSGDFASTTNVSGESSVAMGT |
| | | SGEASGLPEVTLITSEFVEGVTEPTISQELGQRPPVTHTPQLFESSGKVSTAGDISG |
| | | ATPVLPGSGVEVSSVPESSSETSAYPEAGFGASAAPEASREDSGSPDLSETTSAFHE |
| | | ANLERSSGLGVSGSTLTFQEGEASAAPEVSGESTTTSDVGTEAPGLPSATPTASGD |
| | | RTEISGDLSGHTSQLGVVISTSIPESEWTQQTQRPAETHLEIESSSLLYSGEETHTVE |
| | | TATSPTDASIPASPEWKRESESTAAAPARSCAEEPCGAGTCKETEGHVICLCPPGY |
| | | TGEHCNIDQEVCEEGWNKYQGHCYRHFPDRETWVDAERRCREQQSHLSSIVTPE |
| | | EQEFVNNNAQDYQWIGLNDRTIEGDFRWSDGHPMQFENWRPNQPDNFFAAGED |
| | | CVVMIWHEKGEWNDVPCNYHLPFTCKKGTVACGEPPVVEHARTFGQKKDRYEI |
| | | NSLVRYQCTEGFVQRHMPTIRCQPSGHWEEPRITCTD ATTYKRRLQKRSSRHPRR |
| | | SRPSTAH |
| 00745 | 156 | EVQLVESGGGVVQPGGSLRLSCAASGSTFIINVVRWYRRAPGKQRELVATISSGG |
| PEA114F08 | | NANYVDSVRGRFTISRDNSKNTVYLQMNSLRPEDTALYYCNVPTTHYGGVYYG |
| | | PYWGQGTLVTVSSA |
| 00747 | 157 | EVQLVESGGGVVQPGGSLRLSCAASGRTFSSYTMGWFRQAPGKEREFVAAISWS |
| PEA604F02 | | GGRTYYADSVKGRFTISRDNSKNTVYLQMNSLRPEDTALYYCAAYRRRRASSNR |
| | | GLWDYWGQGTLVTVSSA |

In some embodiments, the ADAMTS5 inhibiting polypeptide comprises at least one ISVD that is an amino acid sequence with the (general) structure:

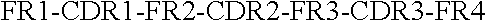

in which FR1 to FR4 refer to framework regions 1 to 4, respectively, and in which CDR1 to CDR3 refer to the complementarity determining regions 1 to 3, respectively. In this embodiment, the at least one ISVD has at least 80%, at least 90%, or at least 95% amino acid identity with at least one of the amino acid sequences of SEQ ID NOs: 2, 116, 19, 1, 3, 6, 16, 17, 10, 11, 9, 5, 4, 7, 117, 8, 12, 13, 14, 15, 18, or amino acids 1-124 of SEQ ID NO: 129 (see Table 2), in which for the purposes of determining the degree of amino acid identity, the amino acid residues that form the 10 CDR sequences are disregarded. In this respect, reference is also made to Table 3, which lists the framework 1 sequences (SEQ ID NOs: 72-84), framework 2 sequences (SEQ ID NOs: 85-94), framework 3 sequences (SEQ ID NOs: 95-113) and framework 4 sequences (SEQ ID NOs: 114-115) of the immunoglobulin single variable domains of SEQ ID NOs: 2, 116, 19, 1, 3, 6, 16, 17, 10, 11, 9, 5, 4, 7, 117, 8, 12, 13, 14, 15, 18, and amino acids 1-124 of SEQ ID NO: 129.

TABLE 2
Name and short description (“ID”), SEQ ID NO:s (“SEQ”) and amino
acid sequences of monovalent and multivalent anti-ADAMTS5 Nanobodies

| ID | SEQ | Sequence |
|---|---|---|
| 2A02 | 5 | EVQLVESGGGLVQPGGSLRLSCAASRRTFSSYVMAWFRQAPGKERE |
| | | FVAAISRSGDSTYYYDSLEGRFTISRDNAKNTVHLQMNSLKPEDTAV |
| | | YICAASRAPSFRTIDAINYYDYWGQGTLVTVSS |
| 2A12 | 1 | EVQLVESGGGLVQAGGSLRLSCAASGRTFSSYAMGWFRQAPGKER |
| | | DFVAGISRSAGRTYYVDSVKGRFTISRDSAKNTVYLQMNRLKPEDT |
| | | AVYYCAADLDPNRIFSRDEAAYWGQGTLVTVSS |
| 2C10 | 9 | EVQLVESGGGLVQAGGSLRLSCATSGFTFSPYYMGWFRQAPGKER |
| | | DFVAAITRSRGTTYYLDSTEGRFTISRDNAKNTMYLQMNSLNPEDT |
| | | AVYYCAAGRSPGDPSRTYLYEYWGQGTLVTVSS |
| 2D07 | 6 | EVQLVESGGGLVQAGGSLRLSCSFSGPGRTFARYAMGWFRQAPGK |
| | | NRDFITGISGSGDSTYYVYPMKDRFTISRDNAKNMVYLQMNALKPE |
| | | DTAVYYCAADREINRIANDKELDFWGQGTLVTVSS |
| 2D12 | 4 | EVQLVESGGGLVQAGDSLRLSCAASGRTFSTYFVGWFRQAPGKERD |
| | | FVAAISRNGARTYYYDSVAGLFTISRDNAKNTVYLQMSSLKPEDTA |
| | | VYYCAAARISPSDPSNEDGYDYWGQGTLVTVSS |
| 2F03 | 2 | EVQLVESGGGLVQAGGSLRLSCAASGRTVSSYAMGWFRLAPGKER |
| | | EFVAGISRSAERTYYVDSLKGRFTISRDSAKNTVYLHMNRLKPEDTA |
| | | VYYCAADLDPNRIFSREEYAYWGQGTLVTVSS |
| 2G01 | 7 | EVQLVESGGGLVQAGGSLRLSCAASGRTTFSSYAMGWFRQAPGKE |
| | | RAFVATIWSGGLTVYADSAKGRFTISRDNAKNTVYLQMNSLRPEDT |
| | | AVYYCAAEAVGTYYTPDGWTYWGQGTLVTVSS |
| 3B02 | 16 | EVQLVESGGGFVQAGGSLRLSCVASRRTISSGTMGWFRQAPGKERE |
| | | FVAAIRWSSGMPYYLDSVMDRFTISRDNAKNTVSLQMNSLQPEDTA |
| | | VYYCAADRSAFRDPSFDVNYEYWGRGTLVTVSS |
| 3B03 | 13 | EVQLVESGGDLVQPGGSLRLSCAASGSDVVVNDMGWYRQAPGKQ |
| | | RELVADITTGGRTNYADSVKGRFTISRDNVKNTVYLQMNSLKPEDT |
| | | AVYYCNAQVGDSDDDVWYAYWGQGTLVTVSS |
| 3D01 | 10 | EVQLVESGGGLVQAGGSLRLSCAPSGFTFSPYYMGWFRQAPGKERD |
| | | FVAAISRSRGTTYYLDSTEGRFTISRDNANDTVYLQMNSLNPEDTAV |
| | | YYCAAGRSPGDPSRTYLYDYWGQGTLVTVSS |
| 3D02 | 11 | EVQLVESGGGLVQAGASLRLSCATSGFTFSPYYMGWFRQAPGKER |
| | | DFVAAISWSRGILYYTDSTEGRFTISRDNAKNTMYLQMDNLNPEDT |
| | | AVYYCAASRSPGDPSRTYLYDYWGQGTLVTVSS |
| 7B11 | 14 | EVQLVESGGGLVQPGGSLRLSCAASGSIFSINVMGWYRQAPGKQRE |
| | | LVAAIISGGRTNYADSVKGRFTISRDNSKNTVYLQMNSLRPEDTAVY |
| | | YCNAEVDAGIYAYGYWGQGTLVTVSS |
| 9A05 | 17 | EVQLVESGGGFVQAGGSLRLSCAASRRTISSGTMGWFRQAPGKERE |
| | | FVAAIRWSSGITFYPDSVEGRFTISGDNAKNTVSLQMNSLKPEDTAV |
| | | YYCAADRSALRDPSFEVNYEYWGRGTLVTVSS |
| 9D03 | 3 | EVQLVESGGGLVQSGGSLRLSCAASGSAVSVNAMAWYRQAPGKQR |
| | | DFVAGISRSAGRTYYTDSVKDRFTIARDSAKNTVYLQMNRLKPEDT |
| | | AVYYCAADLDPNRIFSRDEAAYWGQGTLVTVSS |
| 9D09 | 15 | EVQLVESGGGLVQAGGSLRLSCAASGLTFSSYTMGWFRQAPGQERE |
| | | FVSAISWNTFTTYYVDSVKDRFTVSRDNAKNTLYLRMNSLKPEDTA |
| | | VYYCAAAGGSPRQHEPYEYRVWGQGTLVTVSS |
| 9D10 | 12 | EVQLVESGGGLVQAGGSLRLSCAASGRALSSSIMGWFRQAPGKERE |
| | | FVAAITWSGGRAYYADVSDFEKGRFTISRDNGKNTVNLQMKGLKP |
| | | EDTAVYYCAAALAIPVTMSPHEYPYWGQGTLVTVSS |
| 11B06 | 8 | EVQLVESGGGLVQAGGSLRLSCAASGLTFRRNAMGWFRQAPGKER |
| | | ELLAGINWSGGTTYYVDSVKGRFTISRDNAKNTVDLQMISPKPEDT |
| | | AVYYCAADGDIGTLVNDENPRYWGQGTLVTVSS |
| 13E02 | 18 | EVQLVESGGGLVQAGGSLRLSCVASGSIFSIDAMGWYRQAPGKERE |
| | | LVASVTTGASPNYGDSVTGRFTASRDRAKNALYLQMNSLKPEDTA |
| | | VYYCNLIMTIPGGSQIMYWGQGTLVTVSS |
| 3F04 | 19 | EVQLVESGGGSVQAGGSLRLSCVASGRYPMAWFRQAPGKEREFVA |
| | | GVSWGGDRTYYADSVQGRFTVSRDYAKNTLYLQMNSLKPEDAAV |
| | | YYCAGDPWGRLFRVKDNYSDWGQGTLVTVSS |
| construct 049 | 117 | EVQLVESGGGLVQAGGSLRLSCAASGLTFRRNAMGWFRQAPGKER |
| 11B06: N52 | | ELLAGISWSGGTTYYVDSVKGRFTISRDNAKNTVDLQMISPKPEDTA |
| | | VYYCAADGDIGTLVNDENPRYWGQGTLVTVSS |
| construct093 | 116 | EVQLVESGGGSVQAGGSLRLSCVASGRYPMAWFRQAPGKEREFVA |
| 3F04: N100f | | GVSWGGDRTYYADSVQGRFTVSRDYAKNTLYLQMNSLKPEDAAV |
| Q | | YYCAGDPWGRLFRVKDQYSDWGQGTLVTVSS |
| construct 004 | 120 | EVQLVESGGGLVQAGGSLRLSCAASGRTFSSYAMGWFRQAPGKER |
| 2A12-Alb | | DFVAGISRSAGRTYYVDSVKGRFTISRDSAKNTVYLQMNRLKPEDT |
| | | AVYYCAADLDPNRIFSRDEAAYWGQGTLVTVSSGGGGSGGGGSGG |
| | | GGSGGGGSGGGGSGGGGSGGGGSEVQLVESGGGLVQPGNSLRLSC |
| | | AASGFTFSSFGMSWVRQAPGKGLEWVSSISGSGSDTLYADSVKGRF |
| | | TISRDNAKTTLYLQMNSLRPEDTAVYYCTIGGSLSRSSQGTLVTVSS |
| construct 005 | 121 | EVQLVESGGGLVQAGGSLRLSCSFSGPGRTFARYAMGWFRQAPGK |
| 2D7-Alb | | NRDFITGISGSGDSTYYVYPMKDRFTISRDNAKNMVYLQMNALKPE |
| | | DTAVYYCAADREINRIANDKELDFWGQGTLVTVSSGGGGSGGGGS |
| | | GGGGSGGGGSGGGGSGGGGSGGGGSEVQLVESGGGLVQPGNSLRL |
| | | SCAASGFTFSSFGMSWVRQAPGKGLEWVSSISGSGSDTLYADSVKG |
| | | RFTISRDNAKTTLYLQMNSLRPEDTAVYYCTIGGSLSRSSQGTLVTV |
| | | SS |
| construct 006 | 122 | EVQLVESGGGLVQAGGSLRLSCAASGRTVSSYAMGWFRLAPGKER |
| 2F3-Alb | | EFVAGISRSAERTYYVDSLKGRFTISRDSAKNTVYLHMNRLKPEDTA |
| | | VYYCAADLDPNRIFSREEYAYWGQGTLVTVSSGGGGSGGGGSGGG |
| | | GSGGGGSGGGGSGGGGSGGGGSEVQLVESGGGLVQPGNSLRLSCA |
| | | ASGFTFSSFGMSWVRQAPGKGLEWVSSISGSGSDTLYADSVKGRFTI |
| | | SRDNAKTTLYLQMNSLRPEDTAVYYCTIGGSLSRSSQGTLVTVSS |
| construct 069 | 123 | EVQLVESGGGLVQAGGSLRLSCAASGLTFRRNAMGWFRQAPGKER |
| 049-Alb | | ELLAGISWSGGTTYYVDSVKGRFTISRDNAKNTVDLQMISPKPEDTA |
| | | VYYCAADGDIGTLVNDENPRYWGQGTLVTVSSGGGGSGGGGSGG |
| | | GGSGGGGSGGGGSGGGGSGGGGSEVQLVESGGGLVQPGNSLRLSC |
| | | AASGFTFSSFGMSWVRQAPGKGLEWVSSISGSGSDTLYADSVKGRF |
| | | TISRDNAKTTLYLQMNSLRPEDTAVYYCTIGGSLSRSSQGTLVTVSS |
| construct 070 | 124 | EVQLVESGGGLVQSGGSLRLSCAASGSAVSVNAMAWYRQAPGKQR |
| 9D3-Alb | | DFVAGISRSAGRTYYTDSVKDRFTIARDSAKNTVYLQMNRLKPEDT |
| | | AVYYCAADLDPNRIFSRDEAAYWGQGTLVTVSSGGGGSGGGGSGG |
| | | GGSGGGGSGGGGSGGGGSGGGGSEVQLVESGGGLVQPGNSLRLSC |
| | | AASGFTFSSFGMSWVRQAPGKGLEWVSSISGSGSDTLYADSVKGRF |
| | | TISRDNAKTTLYLQMNSLRPEDTAVYYCTIGGSLSRSSQGTLVTVSS |
| construct 071 | 125 | EVQLVESGGGFVQAGGSLRLSCVASRRTISSGTMGWFRQAPGKERE |
| 3B2-Alb | | FVAAIRWSSGMPYYLDSVMDRFTISRDNAKNTVSLQMNSLQPEDTA |
| | | VYYCAADRSAFRDPSFDVNYEYWGRGTLVTVSSGGGGSGGGGSGG |
| | | GGSGGGGSGGGGSGGGGSGGGGSEVQLVESGGGLVQPGNSLRLSC |
| | | AASGFTFSSFGMSWVRQAPGKGLEWVSSISGSGSDTLYADSVKGRF |
| | | TISRDNAKTTLYLQMNSLRPEDTAVYYCTIGGSLSRSSQGTLVTVSS |
| construct 129 | 126 | EVQLVESGGGLVQAGGSLRLSCAASGRTVSSYAMGWFRLAPGKER |
| 2F3-093-Alb | | EFVAGISRSAERTYYVDSLKGRFTISRDSAKNTVYLHMNRLKPEDTA |
| | | VYYCAADLDPNRIFSREEYAYWGQGTLVTVSSGGGGSGGGGSGGG |
| | | GSGGGGSGGGGSGGGGSGGGGSEVQLVESGGGSVQAGGSLRLSCV |
| | | ASGRYPMAWFRQAPGKEREFVAGVSWGGDRTYYADSVQGRFTVS |
| | | RDYAKNTLYLQMNSLKPEDAAVYYCAGDPWGRLFRVKDQYSDWG |
| | | QGTLVTVSSGGGGSGGGGSGGGGSGGGGSGGGGSGGGGSGGGGSE |
| | | VQLVESGGGLVQPGNSLRLSCAASGFTFSSFGMSWVRQAPGKGLE |
| | | WVSSISGSGSDTLYADSVKGRFTISRDNAKTTLYLQMNSLRPEDTAV |
| | | YYCTIGGSLSRSSQGTLVTVSS |
| construct 130 | 127 | EVQLVESGGGLVQAGGSLRLSCAASGLTFRRNAMGWFRQAPGKER |
| 049-093-Alb | | ELLAGISWSGGTTYYVDSVKGRFTISRDNAKNTVDLQMISPKPEDTA |
| | | VYYCAADGDIGTLVNDENPRYWGQGTLVTVSSGGGGSGGGGSGG |
| | | GGSGGGGSGGGGSGGGGSGGGGSEVQLVESGGGSVQAGGSLRLSC |
| | | VASGRYPMAWFRQAPGKEREFVAGVSWGGDRTYYADSVQGRFTV |
| | | SRDYAKNTLYLQMNSLKPEDAAVYYCAGDPWGRLFRVKDQYSDW |
| | | GQGTLVTVSSGGGGSGGGGSGGGGSGGGGSGGGGSGGGGSGGGGS |
| | | EVQLVESGGGLVQPGNSLRLSCAASGFTFSSFGMSWVRQAPGKGLE |
| | | WVSSISGSGSDTLYADSVKGRFTISRDNAKTTLYLQMNSLRPEDTAV |
| | | YYCTIGGSLSRSSQGTLVTVSS |
| construct 131 | 128 | EVQLVESGGGLVQSGGSLRLSCAASGSAVSVNAMAWYRQAPGKQR |
| 9D3-093-Alb | | DFVAGISRSAGRTYYTDSVKDRFTIARDSAKNTVYLQMNRLKPEDT |
| | | AVYYCAADLDPNRIFSRDEAAYWGQGTLVTVSSGGGGSGGGGSGG |
| | | GGSGGGGSGGGGSGGGGSGGGGSEVQLVESGGGSVQAGGSLRLSC |
| | | VASGRYPMAWFRQAPGKEREFVAGVSWGGDRTYYADSVQGRFTV |
| | | SRDYAKNTLYLQMNSLKPEDAAVYYCAGDPWGRLFRVKDQYSDW |
| | | GQGTLVTVSSGGGGSGGGGSGGGGSGGGGSGGGGSGGGGSGGGGS |
| | | EVQLVESGGGLVQPGNSLRLSCAASGFTFSSFGMSWVRQAPGKGLE |
| | | WVSSISGSGSDTLYADSVKGRFTISRDNAKTTLYLQMNSLRPEDTAV |
| | | YYCTIGGSLSRSSQGTLVTVSS |
| construct | 129 | DVQLVESGGGVVQPGGSLRLSCAASGRTVSSYAMGWFRQAPGKER |
| 577 = 581 | | EFVAGISRSAERTYYVDSLKGRFTISRDNSKNTVYLQMNSLRPEDTA |
| 2F3 *-Alb | | LYYCAADLDPNRIFSREEYAYWGQGTLVTVSSGGGGSGGGGSGGG |
| | | GSGGGGSGGGGSGGGGSGGGGSEVQLVESGGGVVQPGNSLRLSCA |
| | | ASGFTFSSFGMSWVRQAPGKGLEWVSSISGSGSDTLYADSVKGRFTI |
| | | SRDNAKTTLYLQMNSLRPEDTALYYCTIGGSLSRSSQGTLVTVSSA |
| construct 579 | 130 | DVQLVESGGGVVQPGGSLRLSCAASGRTVSSYAMGWFRQAPGKER |
| 2F3 *-093*- | | EFVAGISRSAERTYYVDSLKGRFTISRDNSKNTVYLQMNSLRPEDTA |
| Alb | | LYYCAADLDPNRIFSREEYAYWGQGTLVTVSSGGGGSGGGGSGGG |
| | | GSGGGGSGGGGSGGGGSGGGGSEVQLVESGGGVVQPGGSLRLSCA |
| | | ASGRYPMAWFRQAPGKEREFVAGVSWGGDRTYYADSVKGRFTISR |
| | | DYSKNTLYLQMNSLRPEDTALYYCAGDPFGRLFRVKDQYSDWGQG |
| | | TLVTVSSGGGGSGGGGSGGGGSGGGGSGGGGSGGGGSGGGGSEVQ |
| | | LVESGGGVVQPGNSLRLSCAASGFTFSSFGMSWVRQAPGKGLEWV |
| | | SSISGSGSDTLYADSVKGRFTISRDNAKTTLYLQMNSLRPEDTALYY |
| | | CTIGGSLSRSSQGTLVTVSSA |

*SO (sequence optimized) version

TABLE 3
Sequences for CDRs and frameworks, plus illustrative combinations
as provided in formula I, namely FR1-CDR1-FR2-CDR2-
FR3-CDR3-FR4 (the following terms: “ID” refers to the given SEQ ID NO)

| ID | Nanobody | ID | FR1 | ID | CDR1 | ID | FR2 | ID | CDR2 | ID | FR3 | ID | CDR3 | ID | FR4 |
|---|---|---|---|---|---|---|---|---|---|---|---|---|---|---|---|
| 1 | 02A12 | 72 | EVQLVESGGLV | 20 | GRTFS | 85 | WFRQA | 36 | GISRS | 95 | YVDSVKGRFTISRD | 54 | DLDPN | 114 | WGQGT |
| | | | QAGGSLRLSCA | | SYAMG | | PGKER | | AGRTY | | SAKNTVYLQMNRLK | | RIFSR | | LVTVS |
| | | | AS | | | | DFVA | | | | PEDTAVYYCAA | | DEAAY | | S |
| 2 | 02F03 | 72 | EVQLVESGGLV | 21 | GRTVS | 86 | WFRLA | 37 | GISRS | 96 | YVDSLKGRFTISRD | 55 | DLDPN | 114 | WGQGT |
| | | | QAGGSLRLSCA | | SYAMG | | PGKER | | AERTY | | SAKNTVYLHMNRLK | | RIFSR | | LVTVS |
| | | | AS | | | | EFVA | | | | PEDTAVYYCAA | | EEYAY | | S |
| 3 | 09D03 | 73 | EVQLVESGGLV | 22 | GSAVS | 87 | WYRQA | 36 | GISRS | 97 | YTDSVKDRFTIARD | 54 | DLDPN | 114 | WGQGT |
| | | | QSGGSLRLSCA | | VNAMA | | PGKQR | | AGRTY | | SAKNTVYLQMNRLK | | RIFSR | | LVTVS |
| | | | AS | | | | DFVA | | | | PEDTAVYYCAA | | DEAAY | | S |
| 4 | 02D12 | 74 | EVQLVESGGLV | 23 | GRTFS | 85 | WFRQA | 38 | AISRN | 98 | YDSVAGLFTISRDN | 56 | ARISP | 114 | WGQGT |
| | | | QAGDSLRLSCA | | TYFVG | | PGKER | | GARTY | | AKNTVYLQMSSLKP | | SDPSN | | LVTVS |
| | | | AS | | | | DFVA | | Y | | EDTAVYYCAA | | EDGYD | | S |
| | | | | | | | | | | | | | Y | | |
| 5 | 02A02 | 75 | EVQLVESGGGL | 24 | RRTFS | 88 | WFRQA | 39 | AISRS | 99 | YYDSLEGRFTISRD | 57 | SRAPS | 114 | WGQGT |
| | | | VQPGGSLRLSC | | SYVMA | | PGKER | | GDSTY | | NAKNTVHLQMNSLK | | FRTID | | LVTVS |
| | | | AAS | | | | EFVA | | | | PEDTAVYICAA | | AINYY | | S |
| | | | | | | | | | | | | | DY | | |
| 6 | 02D07 | 76 | EVQLVESGGLV | 25 | GPGRT | 89 | WFRQA | 40 | GISGS | 100 | YVYPMKDRFTISRD | 58 | DREIN | 114 | WGQGT |
| | | | QAGGSLRLSCS | | FARYA | | PGKNR | | GDSTY | | NAKNMVYLQMNALK | | RIAND | | LVTVS |
| | | | FS | | MG | | DFIT | | | | PEDTAVYYCAA | | KELDF | | S |
| 7 | 02G01 | 72 | EVQLVESGGLV | 26 | GRTTF | 90 | WFRQA | 41 | TIWSG | 101 | YADSAKGRFTISRD | 59 | EAVGT | 114 | WGQGT |
| | | | QAGGSLRLSCA | | SSYAM | | PGKER | | GLTV | | NAKNTVYLQMNSLR | | YYTPD | | LVTVS |
| | | | AS | | G | | AFVA | | | | PEDTAVYYCAA | | GWTY | | S |
| 8 | 11B06 | 72 | EVQLVESGGLV | 27 | GLTFR | 91 | WFRQA | 42 | GINWS | 102 | YVDSVKGRFTISRD | 60 | DGDIG | 114 | WGQGT |
| | | | QAGGSLRLSCA | | RNAMG | | PGKER | | GGTTY | | NAKNTVDLQMISPK | | TLVND | | LVTVS |
| | | | AS | | | | ELLA | | | | PEDTAVYYCAA | | ENPRY | | S |
| 117 | 49 | 72 | EVQLVESGGLV | 27 | GLTFR | 91 | WFRQA | 119 | GISWS | 102 | YVDSVKGRFTISRD | 60 | DGDIG | 114 | WGQGT |
| | 11B06: | | QAGGSLRLSCA | | RNAMG | | PGKER | | GGTTY | | NAKNTVDLQMISPK | | TLVND | | LVTVS |
| | N52S | | AS | | | | ELLA | | | | PEDTAVYYCAA | | ENPRY | | S |
| 9 | 02C10 | 77 | EVQLVESGGLV | 28 | GFTFS | 85 | WFRQA | 43 | AITRS | 103 | YLDSTEGRFTISRD | 61 | GRSPG | 114 | WGQGT |
| | | | QAGGSLRLSCA | | PYYMG | | PGKER | | RGTTY | | NAKNTMYLQMNSLN | | DPSRT | | LVTVS |
| | | | TS | | | | DFVA | | | | PEDTAVYYCAA | | YLYEY | | S |
| 10 | 03D01 | 78 | EVQLVESGGGL | 28 | GFTFS | 85 | WFRQA | 44 | AISRS | 104 | YLDSTEGRFTISRD | 62 | GRSPG | 114 | WGQGT |
| | | | VQAGGSLRLSC | | PYYMG | | PGKER | | RGTTY | | NANDTVYLQMNSLN | | DPSRT | | LVTVS |
| | | | APS | | | | DFVA | | | | PEDTAVYYCAA | | YLYDY | | S |
| 11 | 03D02 | 79 | EVQLVESGGGL | 28 | GFTFS | 85 | WFRQA | 45 | AISWS | 105 | YTDSTEGRFTISRD | 63 | SRSPG | 114 | WGQGT |
| | | | VQAGASLRLSC | | PYYMG | | PGKER | | RGILY | | NAKNTMYLQMDNLN | | DPSRT | | LVTVS |
| | | | ATS | | | | DFVA | | | | PEDTAVYYCAA | | YLYDY | | S |
| 12 | 09D10 | 72 | EVQLVESGGLV | 29 | GRALS | 88 | WFRQA | 46 | AITWS | 106 | VSDFEKGRFTISRD | 64 | ALAIP | 114 | WGQGT |
| | | | QAGGSLRLSCA | | SSIMG | | PGKER | | GGRAY | | NGKNTVNLQMKGLK | | VTMSP | | LVTVS |
| | | | AS | | | | EFVA | | YAD | | PEDTAVYYCAA | | HEYPY | | S |
| 13 | 03B03 | 80 | EVQLVESGGDL | 30 | GSDVV | 92 | WYRQA | 47 | DITTG | 107 | YADSVKGRFTISRD | 65 | QVGDS | 114 | WGQGT |
| | | | VQPGGSLRLSC | | VNDMG | | PGKQR | | GRTN | | NVKNTVYLQMNSLK | | DDDVW | | LVTVS |
| | | | AAS | | | | ELVA | | | | PEDTAVYYCNA | | YAY | | S |
| 14 | 07B11 | 75 | EVQLVESGGGL | 31 | GSIFS | 92 | WYRQA | 48 | AIISG | 108 | YADSVKGRFTISRD | 66 | EVDAG | 114 | WGQGT |
| | | | VQPGGSLRLSC | | INVMG | | PGKQR | | GRTN | | NSKNTVYLQMNSLR | | IYAYGY | | LVTVS |
| | | | AAS | | | | ELVA | | | | PEDTAVYYCNA | | | | S |
| 15 | 09D09 | 72 | EVQLVESGGLV | 32 | GLTFS | 93 | WFRQA | 49 | AISWN | 109 | YVDSVKDRFTVSRD | 67 | AGGSP | 114 | WGQGT |
| | | | QAGGSLRLSCA | | SYTMG | | PGQER | | TFTTY | | NAKNTLYLRMNSLK | | RQHEP | | LVTVS |
| | | | AS | | | | EFVS | | | | PEDTAVYYCAA | | YEYRV | | S |
| 16 | 03B02 | 81 | EVQLVESGGGF | 33 | RRTIS | 88 | WFRQA | 50 | AIRWS | 110 | YLDSVMDRFTISRD | 68 | DRSAF | 115 | WGRGT |
| | | | VQAGGSLRLSC | | SGTMG | | PGKER | | SGMPY | | NAKNTVSLQMNSLQ | | RDPSF | | LVTVS |
| | | | VAS | | | | EFVA | | | | PEDTAVYYCAA | | DVNYE | | S |
| | | | | | | | | | | | | | Y | | |
| 17 | 09A05 | 82 | EVQLVESGGGF | 33 | RRTIS | 88 | WFRQA | 51 | AIRWS | 111 | YPDSVEGRFTISGD | 69 | DRSAL | 114 | WGQGT |
| | | | VQAGGSLRLSC | | SGTMG | | PGKER | | SGITF | | NAKNTVSLQMNSLK | | RDPSF | | LVTVS |
| | | | AAS | | | | EFVA | | | | PEDTAVYYCAA | | EVNYE | | S |
| | | | | | | | | | | | | | Y | | |
| 18 | 13E02 | 83 | EVQLVESGGGL | 34 | GSIFS | 94 | WYRQA | 52 | SVTTG | 112 | YGDSVTGRFTASRD | 70 | IMTIP | 114 | WGQGT |
| | | | VQAGGSLRLSC | | IDAMG | | PGKER | | ASPN | | RAKNALYLQMNSLK | | GGSQI | | LVTVS |
| | | | VAS | | | | ELVA | | | | PEDTAVYYCNL | | MY | | S |
| 19 | 03F04 | 84 | EVQLVESGGGS | 35 | GRYPM | 88 | WFRQA | 53 | GVSWG | 113 | YADSVQGRFTVSRD | 71 | DPWGR | 114 | WGQGT |
| | | | VQAGGSLRLSC | | A | | PGKER | | GDRTY | | YAKNTLYLQMNSLK | | LFRVK | | LVTVS |
| | | | VAS | | | | EFVA | | | | PEDAAVYYCAG | | DNYSD | | S |

| | | | | | | | | | | | | | | | |
|---|---|---|---|---|---|---|---|---|---|---|---|---|---|---|---|
| 116 | 93(3F04 | 84 | EVQLVESGGGS | 35 | GRYPM | 88 | WFRQA | 53 | GVSWG | 113 | YADSVQGRFTVSRD | 118 | DPWGR | 114 | WGQGT |
| | N100fQ) | | VQAGGSLRLSC | | A | | PGKER | | GDRTY | | YAKNTLYLQMNSLK | | LFRVK | | LVTVS |
| | | | VAS | | | | EFVA | | | | PEDAAVYYCAG | | DQYSD | | S |

In some embodiments, the ADAMTS5 inhibiting polypeptide comprises four framework regions (FR1 to FR4, respectively) and three complementarity determining regions (CDR1 to CDR3 respectively), in which (i) CDR1 is chosen from the group consisting of SEQ ID NOs: 21, 35, 20, 22, 25, 33, 28, 24, 23, 26, 27, 29, 30, 31, 32 and 34; and amino acid sequences that have 1, 2 or 3 amino acid difference(s) with SEQ ID NOs: 21, 35, 20, 22, 25, 33, 33, 28, 24, 23, 26, 27, 29, 30, 31, 32 and 34; (ii) CDR2 is chosen from the group consisting of SEQ ID NOs: 37, 53, 36, 40, 50, 51, 44, 45, 43, 39, 38, 41, 119, 42, 46, 47, 48, 49 and 52; and amino acid sequences that have 1, 2 or 3 amino acid difference(s) with SEQ ID NOs: 37, 53, 36, 40, 50, 51, 44, 45, 43, 39, 38, 41, 119, 42, 46, 47, 48, 49 and 52; and (iii) CDR3 is chosen from the group consisting of SEQ ID NOs: 55, 118, 71, 54, 58, 68, 69, 62, 63, 61, 57, 56, 59, 60, 64, 65, 66, 67 and 70; and amino acid sequences that have 1, 2, 3 or 4 amino acid difference(s) with SEQ ID NOs: 55, 118, 71, 54, 58, 68, 69, 62, 63, 61, 57, 56, 59, 60, 64, 65, 66, 67 and 70. In some embodiments, the ADAMTS5 inhibiting polypeptide consists essentially of four framework regions (FR1 to FR4, respectively) and three complementarity determining regions (CDR1 to CDR3 respectively), in which (i) CDR1 is chosen from the group consisting of SEQ ID NOs: 21, 35, 20, 22, 25, 33, 28, 24, 23, 26, 27, 29, 30, 31, 32 and 34; and amino acid sequences that have 1, 2 or 3 amino acid difference(s) with SEQ ID NOs: 21, 35, 20, 22, 25, 33, 33, 28, 24, 23, 26, 27, 29, 30, 31, 32 and 34; (ii) CDR2 is chosen from the group consisting of SEQ ID NOs: 37, 53, 36, 40, 50, 51, 44, 45, 43, 39, 38, 41, 119, 42, 46, 47, 48, 49 and 52; and amino acid sequences that have 1, 2 or 3 amino acid difference(s) with SEQ ID NOs: 37, 53, 36, 40, 50, 51, 44, 45, 43, 39, 38, 41, 119, 42, 46, 47, 48, 49 and 52; and (iii) CDR3 is chosen from the group consisting of SEQ ID NOs: 55, 118, 71, 54, 58, 68, 69, 62, 63, 61, 57, 56, 59, 60, 64, 65, 66, 67 and 70; and amino acid sequences that have 1, 2, 3 or 4 amino acid difference(s) with SEQ ID NOs: 55, 118, 71, 54, 58, 68, 69, 62, 63, 61, 57, 56, 59, 60, 64, 65, 66, 67 and 70. Illustrative framework sequences are disclosed in Table 3 and can be used in an ISVD as described herein. In some embodiments, the CDRs depicted in Table 3 are matched with the respective framework regions of the same ISVD construct.

In some embodiments, the ADAMTS5 inhibiting polypeptide comprises an ISVD chosen from the group of ISVDs, wherein: CDR1 is chosen from the group consisting of SEQ ID NOS: 21, 35, 20, 22, 25, 33, 28, 24, 23, 26, 27, 29, 30, 31, 32 and 34; CDR2 is chosen from the group consisting of SEQ ID NOs: 37, 53, 36, 40, 50, 51, 44, 45, 43, 39, 38, 41, 119, 42, 46, 47, 48, 49 and 52; and CDR3 is chosen from the group consisting of SEQ ID NOs: 55, 118, 71, 54, 58, 68, 69, 62, 63, 61, 57, 56, 59, 60, 64, 65, 66, 67 and 70.

In some embodiments, the ADAMTS5 inhibiting polypeptide comprises an ISVD chosen from the group of ISVDs, wherein:
CDR1 is SEQ ID NO: 21, CDR2 is SEQ ID NO: 37 and CDR3 is SEQ ID NO: 55;
CDR1 is SEQ ID NO: 35, CDR2 is SEQ ID NO: 53 and CDR3 is SEQ ID NO: 118;
CDR1 is SEQ ID NO: 35, CDR2 is SEQ ID NO: 53 and CDR3 is SEQ ID NO: 71;
CDR1 is SEQ ID NO: 20, CDR2 is SEQ ID NO: 36 and CDR3 is SEQ ID NO: 54;
CDR1 is SEQ ID NO: 22, CDR2 is SEQ ID NO: 36 and CDR3 is SEQ ID NO: 54;
CDR1 is SEQ ID NO: 25, CDR2 is SEQ ID NO: 40 and CDR3 is SEQ ID NO: 58;
CDR1 is SEQ ID NO: 33, CDR2 is SEQ ID NO: 50 and CDR3 is SEQ ID NO: 68;
CDR1 is SEQ ID NO: 33, CDR2 is SEQ ID NO: 51 and CDR3 is SEQ ID NO: 69;
CDR1 is SEQ ID NO: 28, CDR2 is SEQ ID NO: 44 and CDR3 is SEQ ID NO: 62;
CDR1 is SEQ ID NO: 28, CDR2 is SEQ ID NO: 45 and CDR3 is SEQ ID NO: 63;
CDR1 is SEQ ID NO: 28, CDR2 is SEQ ID NO: 43 and CDR3 is SEQ ID NO: 61;
CDR1 is SEQ ID NO: 24, CDR2 is SEQ ID NO: 39 and CDR3 is SEQ ID NO: 57;
CDR1 is SEQ ID NO: 23, CDR2 is SEQ ID NO: 38 and CDR3 is SEQ ID NO: 56;
CDR1 is SEQ ID NO: 26, CDR2 is SEQ ID NO: 41 and CDR3 is SEQ ID NO: 59;
CDR1 is SEQ ID NO: 27, CDR2 is SEQ ID NO: 119 and CDR3 is SEQ ID NO: 60;
CDR1 is SEQ ID NO: 27, CDR2 is SEQ ID NO: 42 and CDR3 is SEQ ID NO: 60;
CDR1 is SEQ ID NO: 29, CDR2 is SEQ ID NO: 46 and CDR3 is SEQ ID NO: 64;
CDR1 is SEQ ID NO: 30, CDR2 is SEQ ID NO: 47 and CDR3 is SEQ ID NO: 65;
CDR1 is SEQ ID NO: 31, CDR2 is SEQ ID NO: 48 and CDR3 is SEQ ID NO: 66;
CDR1 is SEQ ID NO: 32, CDR2 is SEQ ID NO: 49 and CDR3 is SEQ ID NO: 67; and
CDR1 is SEQ ID NO: 34, CDR2 is SEQ ID NO: 52 and CDR3 is SEQ ID NO: 70.

In some embodiments, the ADAMTS5 inhibiting polypeptide comprises an ISVD in which CDR1 is SEQ ID NO: 21, CDR2 is SEQ ID NO: 37 and CDR3 is SEQ ID NO: 55.

In some embodiments, the ADAMTS5 inhibiting polypeptide comprises an ISVD chosen from the group consisting of SEQ ID NOs: 2, 116, 19, 1, 3, 6, 16, 17, 10, 11, 9, 5, 4, 7, 8, 117, 12, 13, 14, 15, 18, and amino acids 1-124 of SEQ ID NO: 129.

In some embodiments, the ADAMTS5 inhibiting polypeptide comprises two or more ISVDs which specifically bind ADAMTS5, wherein (a) at least a first ISVD specifically binds a first antigenic determinant, epitope, part, domain, subunit or conformation of ADAMTS5; and wherein (b) at least a second ISVD specifically binds a second antigenic determinant, epitope, part, domain, subunit or conformation of ADAMTS5, different from the first antigenic determinant epitope, part, domain, subunit or conformation, respectively. In some embodiments, the first ISVD specifically binding ADAMTS5 is chosen from the group consisting of SEQ ID NOs: 2, 1, 3, 6, 16, 17, 10, 11, 9, 5, 4, 7, 8, 117, 12, 13, 14, 15, 18, and amino acids 1-124 of SEQ ID NO: 129. In some embodiments, the second ISVD specifically binding ADAMTS5 is SEQ ID NO: 118 or 19. In some embodiments, the ADAMTS5 inhibiting polypeptide comprising two or more ISVDs which specifically bind ADAMTS5 is chosen from the group consisting of SEQ ID NO: 126 (clone 129 2F3-093-Alb), SEQ ID NO: 127 (clone 130 049-093-Alb) and SEQ ID NO: 128 (clone 131 9D3-093-Alb).

In some embodiments, the ADAMTS5 inhibiting polypeptide comprises at least one ISVD specifically binding ADAMTS5 as described above, and at least one ISVD binding a serum albumin, such as human serum albumin. In some embodiments, the ISVD binding serum albumin comprises or essentially consists of four framework regions (FR1 to FR4, respectively) and three complementarity determining regions (CDR1 to CDR3 respectively), in which CDR1 is SFGMS (SEQ ID NO: 146), CDR2 is SISGSGSDTLYADSVKG (SEQ ID NO: 147) and CDR3 is GGSLSR (SEQ ID NO: 148). In some embodiments, the ISVD binding human serum albumin is chosen from the group consisting of Alb8 (SEQ ID NO: 131), Alb23 (SEQ ID NO: 132), Alb129 (SEQ ID NO: 133), Alb132 (SEQ ID NO: 134), Alb11 (SEQ ID NO: 135), Alb11 (S112K)-A (SEQ ID NO: 136), Alb82 (SEQ ID NO: 137), Alb82-A (SEQ ID NO: 138), Alb82-AA (SEQ ID NO: 139), Alb82-AAA (SEQ ID NO: 140), Alb82-G (SEQ ID NO: 141), Alb82-GG (SEQ ID NO: 142), Alb82-GGG (SEQ ID NO: 143), Alb92 (SEQ ID NO: 144), or Alb223 (SEQ ID NO: 145). (see Table 4)

TABLE 4
Serum albumin binding ISVD sequences (“ID” refers to the SEQ ID NO
as used herein), including the CDRsequences

| Name | ID | Amino acid sequence |
|---|---|---|
| Alb8 | 131 | EVQLVESGGGLVQPGNSLRLSCAASGFTFSSFGMSWVRQAPGKGLEWVSSISGSGSD |
| | | TLYADSVKGRFTISRDNAKTTLYLQMNSLRPEDTAVYYCTIGGSLSRSSQGTLVTVSS |
| Alb23 | 132 | EVQLLESGGGLVQPGGSLRLSCAASGFTFRSFGMSWVRQAPGKGPEWVSSISGSGSD |
| | | TLYADSVKGRFTISRDNSKNTLYLQMNSLRPEDTAVYYCTIGGSLSRSSQGTLVTVSS |
| Alb129 | 133 | EVQLVESGGGVVQPGNSLRLSCAASGFTFSSFGMSWVRQAPGKGLEWVSSISGSGSD |
| | | TLYADSVKGRFTISRDNAKTTLYLQMNSLRPEDTATYYCTIGGSLSRSSQGTLVTVSS |
| | | A |
| Alb132 | 134 | EVQLVESGGGVVQPGGSLRLSCAASGFTFRSFGMSWVRQAPGKGPEWVSSISGSGSD |
| | | TLYADSVKGRFTISRDNSKNTLYLQMNSLRPEDTATYYCTIGGSLSRSSQGTLVTVSS |
| | | A |
| Alb11 | 135 | EVQLVESGGGLVQPGNSLRLSCAASGFTFSSFGMSWVRQAPGKGLEWVSSISGSGSD |
| | | TLYADSVKGRFTISRDNAKTTLYLQMNSLRPEDTAVYYCTIGGSLSRSSQGTLVTVSS |
| Alb11 | 136 | EVQLVESGGGLVQPGNSLRLSCAASGFTFSSFGMSWVRQAPGKGLEWVSSISGSGSD |
| (S112K)-A | | TLYADSVKGRFTISRDNAKTTLYLQMNSLRPEDTAVYYCTIGGSLSRSSQGTLVKVSS |
| | | A |
| Alb82 | 137 | EVQLVESGGGVVQPGNSLRLSCAASGFTFSSFGMSWVRQAPGKGLEWVSSISGSGSD |
| | | TLYADSVKGRFTISRDNAKTTLYLQMNSLRPEDTALYYCTIGGSLSRSSQGTLVTVSS |
| Alb82-A | 138 | EVQLVESGGGVVQPGNSLRLSCAASGFTFSSFGMSWVRQAPGKGLEWVSSISGSGSD |
| | | TLYADSVKGRFTISRDNAKTTLYLQMNSLRPEDTALYYCTIGGSLSRSSQGTLVTVSS |
| | | A |
| Alb82-AA | 139 | EVQLVESGGGVVQPGNSLRLSCAASGFTFSSFGMSWVRQAPGKGLEWVSSISGSGSD |
| | | TLYADSVKGRFTISRDNAKTTLYLQMNSLRPEDTALYYCTIGGSLSRSSQGTLVTVSS |
| | | AA |
| Alb82-AAA | 140 | EVQLVESGGGVVQPGNSLRLSCAASGFTFSSFGMSWVRQAPGKGLEWVSSISGSGSD |
| | | TLYADSVKGRFTISRDNAKTTLYLQMNSLRPEDTALYYCTIGGSLSRSSQGTLVTVSS |
| | | AAA |
| Alb82-G | 141 | EVQLVESGGGVVQPGNSLRLSCAASGFTFSSFGMSWVRQAPGKGLEWVSSISGSGSD |
| | | TLYADSVKGRFTISRDNAKTTLYLQMNSLRPEDTALYYCTIGGSLSRSSQGTLVTVSS |
| | | G |
| Alb82-GG | 142 | EVQLVESGGGVVQPGNSLRLSCAASGFTFSSFGMSWVRQAPGKGLEWVSSISGSGSD |
| | | TLYADSVKGRFTISRDNAKTTLYLQMNSLRPEDTALYYCTIGGSLSRSSQGTLVTVSS |
| | | GG |
| Alb82-GGG | 143 | EVQLVESGGGVVQPGNSLRLSCAASGFTFSSFGMSWVRQAPGKGLEWVSSISGSGSD |
| | | TLYADSVKGRFTISRDNAKTTLYLQMNSLRPEDTALYYCTIGGSLSRSSQGTLVTVSS |
| | | GGG |
| Alb92 | 144 | EVQLVESGGGVVQPGGSLRLSCAASGFTFRSFGMSWVRQAPGKGPEWVSSISGSGSD |
| | | TLYADSVKGRFTISRDNSKNTLYLQMNSLRPEDTALYYCTIGGSLSRSSQGTLVTVSS |
| Alb223 | 145 | EVQLVESGGGVVQPGGSLRLSCAASGFTFRSFGMSWVRQAPGKGPEWVSSISGSGSD |
| | | TLYADSVKGRFTISRDNSKNTLYLQMNSLRPEDTALYYCTIGGSLSRSSQGTLVTVSS |
| | | A |
| ALB CDR1 | 146 | SFGMS |
| ALB CDR2 | 147 | SISGSGSDTLYADSVKG |
| ALB CDR3 | 148 | GGSLSR |

In some embodiments, the ADAMTS5 inhibiting polypeptide comprises at least one ISVD binding ADAMTS5 and an ISVD binding serum albumin. For example, in some embodiments, the ISVD binding ADAMTS5 and an ISVD binding serum albumin are chosen from the group consisting of SEQ ID NO: 129 (clone 577 2F3*-Alb), SEQ ID NO: 130 (clone 579 2F3*-093-Alb), SEQ ID NO: 120 (clone 4 2A12-Alb), SEQ ID NO: 121 (clone 5 2D7-Alb), SEQ ID NO: 122 (clone 6 2F3-Alb), SEQ ID NO: 123 (clone 69 049-Alb), SEQ ID NO: 124 (clone 70 9D3-Alb), SEQ ID NO: 125 (clone 71 3B2-Alb), SEQ ID NO: 126 (clone 129 2F3-093-Alb), SEQ ID NO: 127 (clone 130 049-093-Alb), and SEQ ID NO: 128 (clone 131 9D3-093-Alb) (see Table 2).

In some embodiments, the ADAMTS5 inhibiting polypeptide comprises at least two ISVDs directly linked to each other or linked via a linker. In embodiments wherein the at least two ISVDs are linked via a linker, the linker may optionally be chosen from the group consisting of SEQ ID NOs: 158 to 174 (i.e. SEQ ID NO: 158, 159, 160, 161, 162, 163, 164, 165, 166, 167, 168, 169, 170, 171, 172, 173 and 174). In some embodiments, the linker comprises the sequence of SEQ ID NO: 169. (see Table 5).

TABLE 5
Illustrative Linker sequences (“ID” refers to the SEQ ID NO as used herein)

| Name | ID | Amino acid sequence |
|---|---|---|
| A3 | 158 | AAA |
| 5GS linker | 159 | GGGGS |
| 7GS linker | 160 | SGGSGGS |
| 8GS linker | 161 | GGGGGGGS |
| 9GS linker | 162 | GGGGSGGGS |
| 10GS linker | 163 | GGGGSGGGGS |
| 15GS linker | 164 | GGGGSGGGGSGGGGS |
| 18GS linker | 165 | GGGGSGGGGSGGGGGGGS |
| 20GS linker | 166 | GGGGSGGGGSGGGGSGGGGS |
| 25GS linker | 167 | GGGGSGGGGSGGGGSGGGGSGGGGS |
| 30GS linker | 168 | GGGGSGGGGSGGGGSGGGGSGGGGSGGGGS |
| 35GS linker | 169 | GGGGSGGGGSGGGGSGGGGSGGGGSGGGGSGGGGS |
| 40GS linker | 170 | GGGGSGGGGSGGGGSGGGGSGGGGSGGGGSGGGGSGGGGS |
| G1 hinge | 171 | EPKSCDKTHTCPPCP |
| 9GS-G1 hinge | 172 | GGGGSGGGSEPKSCDKTHTCPPCP |
| Llama upper long hinge | 173 | EPKTPKPQPAAA |
| region | | |
| G3 hinge | 174 | ELKTPLGDTTHTCPRCPEPKSCDTPPPCPRCPEPKSCDTPPPCP |
| | | RCPEPKSCDTPPPCPRCP |

In some embodiments, the ADAMTS5 inhibiting polypeptide has at least 80%, at least 90%, at least 95% or 100% sequence identity with any of SEQ ID NOs: 1-19 (i.e. SEQ ID NO: 1, 2, 3, 4, 5, 6, 7, 8, 9, 10, 11, 12, 13, 14, 15, 16, 17, 18 and 19), 116-117, or 120-130 (i.e. SEQ ID NOs: 120, 121, 122, 123, 124, 125, 126, 127, 128, 129 and 130).

In some embodiments, the ADAMTS5 inhibiting polypeptide comprises at least two ISVDs, wherein the at least two ISVDs can be the same or different, but of which at least one ISVD is directed against ADAMTS5. In some embodiments, the ADAMTS5 inhibiting polypeptide consists essentially of at least two ISVDs, wherein the at least two ISVDs can be the same or different, but of which at least one ISVD is directed against ADAMTS5. In some embodiments, the ISVD binding ADAMTS5 is chosen from the group consisting of SEQ ID NOs: 2, 116, 19, 1, 3, 6, 16, 17, 10, 11, 9, 5, 4, 7, 117, 8, 12, 13, 14, 15, 18, and amino acids 1-124 of SEQ ID NO: 129. Illustrative bivalent, bispecific ADAMTS5 inhibiting polypeptides are shown in Table 2 (SEQ ID NOs: 120-130, i.e. SEQ ID NO: 120, 121, 122, 123, 124, 125, 126, 127, 128, 129 and 130). In some embodiments, the bivalent, bispecific ADAMTS5 polypeptides comprise SEQ ID NOs: 129 and 130.

### Methods of Treatment

The present disclosure provides methods for the treatment of a disease or a disorder that results at least partially from ADAMTS5 activity. The methods comprise administration of an effective amount (e.g., a therapeutically effective amount) of an ADAMTS5 inhibiting polypeptide to a subject in need thereof. In some embodiments, the subject is a human subject. Examples of diseases or disorders that may be treated with an ADAMTS5 inhibiting polypeptide include, but are not limited to, arthropathies and chondrodystrophies, arthritic disease (such as osteoarthritis, rheumatoid arthritis, gouty arthritis, psoriatic arthritis), traumatic rupture or detachment, achondroplasia, costochondritis, Spondyloepimetaphyseal dysplasia, spinal disc herniation, lumbar disk degeneration disease, degenerative joint disease, relapsing polychondritis, osteochondritis dissecans and aggrecanopathies. In some embodiments, the disease or disorder is an arthritic disease. In some embodiments, the disease or disorder is osteoarthritis. In some embodiments, the osteoarthritis is knee, hand, hip, or spine osteoarthritis.

In embodiments where the disease or disorder is osteoarthritis, treatment with an ADAMTS5 inhibiting compound according to one of the dosing regimens described herein is expected to slow or halt the progress of osteoarthritis and reduce or eliminate symptoms associated with osteoarthritis as compared to treatment with placebo. In one non-limiting example, treatment may reduce pain as measured by the Knee Injury and Osteoarthritis Outcome Score (KOOS) or the Western Ontario and McMaster Universities Osteoarthritis Index (WOMAC) score. In some embodiments, treatment may reduce stiffness associated with osteoarthritis. In some embodiments, treatment may result in the substantial maintenance of joint structure based on cartilage volume as determined by automated segmentation in MRI. In some embodiments, treatment according to one of the presently described dosing regimens may improve or maintain (e.g., prevent further decrease) the function in the affected joint. In some embodiments, treatment according to one of the presently described dosing regimens may prolong the survival of the joint affected with osteoarthritis and/or increase the subject's quality of life. In some embodiments, treatment according to a dosage regimen as described herein may prevent or delay the need for joint replacement surgery. Treatment according to the dosing regimens described below may continue until such time as the human subject no longer receives a therapeutic benefit.

In some embodiments, treatment according to the described dosing regimen will result in a decrease in the formation of ARGS fragments. ARGS fragments are aggrecan degradation neo-epitopes produced through ADAMTS5-induced cleavage of aggrecan. An illustrative sequence of an ARGS fragment is provided as SEQ ID NO: 175. Aggrecan fragments are retained in the cartilage matrix and are subsequently cleaved by matrix metalloproteases (MMPs), releasing a 32-amino acid fragment. This 32-amino acid fragment has been identified in synovial fluid from osteoarthritis patients and has been shown to activate Toll-like receptor-2 (TLR-2). Activation of TLR-2 has been shown to exacerbate inflammation and pain in the joint affected by osteoarthritis. Consequently, suppression of ARGS fragments would be expected to result in a decrease in inflammation and pain in the joint affected by osteoarthritis, e.g., the knee joint.

The ADAMTS5 inhibiting polypeptide may be administered according to any known administration method. In some embodiments, the ADAMTS5 inhibiting polypeptide is administered subcutaneously. Other possible routes of administration include, e.g., intradermal, intramuscular, intravenous, and intra-articular. The ADAMTS5 inhibiting polypeptide may also be administered according to any known means for administering a therapeutic to a patient, including, but not limited to, a pre-filled syringe, a vial and syringe, an injection pen, an autoinjector, an i.v. drip and bag, a pump, a patch pump, etc. With such items, a patient may self-administer the drug (i.e., administer the drug on their own behalf) or a physician may administer the drug.

### Doses and Dosing Regimens

The methods of the present disclosure may, in some embodiments, comprise administering the ADAMTS5 inhibiting polypeptide according to a dosing regimen. In some embodiments, the dosing regimen for an ADAMTS5 inhibiting polypeptide can be initiated by administering the ADAMTS5 inhibiting polypeptide to the subject one or more times. In some embodiments, following administration of an initial dose, the subject may then be administered one or more additional therapeutically effective doses of the ADAMTS5 inhibiting polypeptide. Subsequent therapeutically effective doses can be administered, for example, according to a weekly dosing schedule, or once every two weeks (biweekly), once every three weeks, once every four weeks, once every 6 weeks, once every two months (bimonthly), once every 10 weeks, once every three months, once every 14 weeks, once every four months, once every 18 weeks, once every five months, once every 22 weeks, once every six months, once every 7 months, once every 8 months, once every 9 months, once every 10 months, once every 11 months, or once every 12 months. In some embodiments, the subsequent doses are administered monthly. The therapeutically effective amount of the ADAMTS5 inhibiting polypeptide may be the same in each dose, or may vary from dose to dose.

In some embodiments, a dosing regimen comprises administration of 300 mg of an ADAMTS5 inhibiting polypeptide to a human subject once weekly. In some embodiments, a dosing regimen comprises administration of 300 mg of an ADAMTS5 inhibiting polypeptide to a human subject biweekly (i.e., once every two weeks). In some embodiments, a dosing regimen comprises administration of 300 mg of an ADAMTS5 inhibiting polypeptide to a human subject once monthly. In some embodiments, a dosing regimen may comprise administration of 300 mg of an ADAMTS5 inhibiting polypeptide to a human subject bimonthly (i.e., once every two months). In some embodiments, a dosing regimen may comprise administration of 300 mg of an ADAMTS5 inhibiting polypeptide to a human subject once every three months.

In some embodiments, a dosing regimen comprises administration of 150 mg of an ADAMTS5 inhibiting polypeptide to a human subject weekly (i.e., about once every seven days). In some embodiments, a dosing regimen comprises administration of 150 mg of an ADAMTS5 inhibiting polypeptide to a human subject biweekly (once every two weeks). In some embodiments, a dosing regimen comprises administration of 150 mg of an ADAMTS5 inhibiting polypeptide to a human subject once monthly. In some embodiments, a dosing regimen may comprise administration of 150 mg of an ADAMTS5 inhibiting polypeptide to a human subject bimonthly (once every two months). In some embodiments, a dosing regimen may comprise administration of 150 mg of an ADAMTS5 inhibiting polypeptide to a human subject once every three months.

In some embodiments, a dosing regimen comprises administration of 75 mg of an ADAMTS5 inhibiting polypeptide to a human subject weekly. In some embodiments a dosing regimen comprises administration of 75 mg of an ADAMTS5 inhibiting polypeptide to a human subject biweekly (i.e., once every two weeks). In some embodiments, a dosing regimen comprises administration of 75 mg of an ADAMTS5 inhibiting polypeptide to a human subject once monthly. In some embodiments, a dosing regimen may comprise administration of 75 mg of an ADAMTS5 inhibiting polypeptide to a human subject bimonthly (i.e., once every two months). In some embodiments, a dosing regimen may comprise administration of 75 mg of an ADAMTS5 inhibiting polypeptide to a human subject once every three months.

Alternatively, in some embodiments, the ADAMTS5 inhibiting polypeptide dosing regimen involves a “loading regimen” and a “maintenance regimen.” The “loading regimen” may comprise one or more loading doses comprising an initial therapeutically effective dose of the ADAMTS5 inhibiting polypeptide that is administered to the subject one or several times. In embodiments wherein the “loading regimen” comprises one or more doses, the doses are generally administered once a week, once every two weeks (biweekly), once every three weeks, or once monthly. In some embodiments, the dose or doses administered during the “loading regimen” are higher than the dose or doses administered in the “maintenance regimen.” In some embodiments, the dose or doses administered in the “loading regimen” are the same as those administered during the “maintenance regimen” with the difference being that the “loading regimen” doses are administered on a more frequent basis.

Following completion of the “loading regimen,” the subsequent dose or doses are administered according to a “maintenance regimen,” wherein the therapeutically effective dose is administered once every two weeks (biweekly), once a month, once every 6 weeks, once every two months (bimonthly), once every 10 weeks, once every three months, once every 14 weeks, once every four months, once every 18 weeks, once every five months, once every 22 weeks, once every six months, once every 7 months, once every 8 months, once every 9 months, once every 10 months, once every 11 months, or once every 12 months. In some embodiments comprising a “maintenance regimen,” the therapeutically effective doses of the ADAMTS5 inhibiting polypeptide are lower than the therapeutically effective doses administered during the “loading regimen.” In some embodiments, the dose or doses administered in the “maintenance regimen” are the same as those administered during the “loading regimen,” with the difference being that the “maintenance regimen” doses are administered on a less frequent basis.

In some embodiments, the ADAMTS5 inhibiting polypeptide is administered at a dose of 300 mg once a week for two, four, six, eight, ten, or twelve weeks during the loading regimen, and subsequently dosed at 300 mg once a month during the maintenance regimen. In some embodiments, the ADAMTS5 inhibiting polypeptide is administered at a dose of 300 mg once a week for two, four, six, eight, ten, or twelve weeks during the loading regimen, and subsequently dosed at 150 mg once a month during the maintenance regimen. In some embodiments, the ADAMTS5 inhibiting polypeptide is administered at a dose of 300 mg once every two weeks (biweekly) for four, six, eight, ten, or twelve weeks during the loading regimen, and subsequently dosed at 300 mg once a month during the maintenance regimen. In some embodiments, the ADAMTS5 inhibiting polypeptide is administered at a dose of 300 mg once every two weeks (biweekly) for four, six, eight, ten, or twelve weeks during the loading regimen, and subsequently dosed at 150 mg once a month during the maintenance regimen. In some embodiments, the ADAMTS5 inhibiting polypeptide is administered at a dose of 300 mg once a month for one, two, three, four, five, or six months during the loading regimen, and subsequently dosed at 150 mg once a month during the maintenance regimen. In some embodiments, the ADAMTS5 inhibiting polypeptide is administered at a dose of 300 mg once a month for one, two, three, four, five, or six months during the loading regimen, and subsequently dosed at 300 mg once every two months (bimonthly) during the maintenance regimen. In some embodiments, the ADAMTS5 inhibiting polypeptide is administered at a dose of 300 mg once a month for one, two, three, four, five, or six months during the loading regimen, and subsequently dosed at 150 mg once every two months (bimonthly) during the maintenance regimen.

In some embodiments, the ADAMTS5 inhibiting polypeptide is administered at a dose of 150 mg once a week for two, four, six, eight, ten, or twelve weeks during the loading regimen, and subsequently dosed at 150 mg once a month during the maintenance regimen. In some embodiments, the ADAMTS5 inhibiting polypeptide is administered at a dose of 150 mg once a week for two, four, six, eight, ten, or twelve weeks during the loading regimen, and subsequently dosed at 75 mg once a month during the maintenance regimen. In some embodiments, the ADAMTS5 inhibiting polypeptide is administered at a dose of 150 mg once every two weeks (biweekly) for four, six, eight, ten, or twelve weeks during the loading regimen, and subsequently dosed at 150 mg once a month during the maintenance regimen. In some embodiments, the ADAMTS5 inhibiting polypeptide is administered at a dose of 150 mg once every two weeks (biweekly) for four, six, eight, ten, or twelve weeks during the loading regimen, and subsequently dosed at 75 mg once a month during the maintenance regimen. In some embodiments, the ADAMTS5 inhibiting polypeptide is administered at a dose of 150 mg once a month for one, two, three, four, five, or six months during the loading regimen, and subsequently dosed at 75 mg once a month during the maintenance regimen. In some embodiments, the ADAMTS5 inhibiting polypeptide is administered at a dose of 150 mg once a month for one, two, three, four, five, or six months during the loading regimen, and subsequently dosed at 150 mg once every two months (bimonthly) during the maintenance regimen. In some embodiments, the ADAMTS5 inhibiting polypeptide is administered at a dose of 150 mg once a month for one, two, three, four, five, or six months during the loading regimen, and subsequently dosed at 75 mg once every two months (bimonthly) during the maintenance regimen.

In some embodiments, the ADAMTS5 inhibiting polypeptide is administered at a dose of 75 mg once a week for two, four, six, eight, ten, or twelve weeks during the loading regimen, and subsequently dosed at 75 mg once a month during the maintenance regimen. In some embodiments, the ADAMTS5 inhibiting polypeptide is administered at a dose of 75 mg once every two weeks (biweekly) for four, six, eight, ten, or twelve weeks during the loading regimen, and subsequently dosed at 75 mg once a month during the maintenance regimen. In some embodiments, the ADAMTS5 inhibiting polypeptide is administered at a dose of 75 mg once a month for one, two, three, four, five, or six months during the loading regimen, and subsequently dosed at 75 mg once every two months (bimonthly) during the maintenance regimen.

In some embodiments, the dosing regimens for the ADAMTS5 inhibiting polypeptide comprise one or more dosing cycles. The term “dosing cycle,” as used herein, means administering a drug for a period of time (i.e., the dosing period) followed by a resting period before administration of the drug is resumed. A dosing cycle begins with the first administration of the drug in that cycle. The term “resting period,” as used herein, refers to a period of time during which the subject is not given a drug (i.e., a period of time wherein the treatment with a drug is withheld). For example, if a drug is given on a daily, weekly, or monthly basis, there would be rest period if the administration is discontinued for some time, e.g., for some number of days, weeks, or months. The dosing period and/or the resting period of the dosing cycle can be the same or different between cycles. For example, if the dosing period is once weekly the resting period may be one week or more than one week. It is contemplated that the dose of the drug administered can be the same or different between cycles.

In some embodiments, the dosing cycle comprises one or more months, e.g., one month, two months, three months, four months, five months, six months, seven months, eight months, nine months, ten months, eleven months, twelve months, etc. A twelve month dosing cycle may also be referred to as a yearly dosing cycle. In some embodiments, the dosing cycle is a six-month dosing cycle.

Each dosing cycle may comprise the administration of one or more doses during the dosing period. In some embodiments, the dosing cycle comprises the administration of one dose during the dosing period. In some embodiments, the dosing cycle comprises administration of two, three, four, five, six, seven, eight, nine, ten, eleven, twelve, etc. doses during the dosing period. In some embodiments, each dosing cycle in the dosing regimen comprises the same number of doses. For example, in a dosing regimen comprising three dosing cycles, each dosing cycle may comprise the administration of four doses of the ADAMTS5 inhibiting polypeptide. It is also contemplated that in some embodiments the number of doses administered may vary from dosing cycle to dosing cycle within the same dosing regimen. For example, in a dosing regimen comprising three dosing cycles, dosing cycle 1 may comprise administration of three doses of an ADAMTS5 inhibiting polypeptide, and dosing cycles 2 and 3 may comprise administration of one dose of an ADAMTS5 inhibiting polypeptide.

In embodiments comprising administration of two or more doses during the dosing period, the dose may be administered during consecutive periods (weeks or months). In embodiments comprising administration of two or more doses during the dosing period, the doses may be administered in alternating periods of time, e.g., every other week or every other month. In embodiments comprising alternating doses, the resting period would not begin until the final dose of the dosing period has been administered. For example, in a four-week dosing cycle comprising two doses administered in alternate weeks, the doses would be administered in week one and week three, and the resting period would be week four.

In some embodiments, the resting period is of the same period of time as the dosing period. For example, if the dosing period is one month then the resting period is one month. In some embodiments, the resting period is of a different period of time than the dosing period. For example, if the dosing period is one month then the resting period may be one week or two months. It is contemplated that the length of the dosing period and/or the length of the resting period may vary from dosing cycle to dosing cycle within the same dosing regimen.

In some embodiments, the ADAMTS5 inhibiting polypeptide is administered in a dosing regimen comprising one or more six-month dosing cycles. In this embodiment, 300 mg of the ADAMTS5 inhibiting polypeptide is administered once a month for three consecutive months followed by a three-month resting period. In some embodiments, the six-month dosing cycle comprises administering 300 mg doses of the ADAMTS5 inhibiting polypeptide once a month for five consecutive months followed by a one-month resting period. In an alternative embodiment, 300 mg doses of the ADAMTS5 inhibiting polypeptide are administered once a week for four, six, eight, ten, or twelve weeks with the remaining time in the six-month cycle a rest period. In some embodiments, 300 mg doses of the ADAMTS5 inhibiting polypeptide are administered at a frequency of once a week for four weeks and then once a month for four months followed a one-month resting period. In some embodiments, 300 mg doses of the ADAMTS5 inhibiting polypeptide are administered at a frequency of once a week for four weeks and then four 150 mg doses are administered once a month for four months followed by a one-month resting period.

In some embodiments, the ADAMTS5 inhibiting polypeptide is administered in a dosing regimen comprising one or more six-month dosing cycles. In this embodiment, 150 mg of the ADAMTS5 inhibiting polypeptide is administered once a month for three consecutive months followed by a three-month resting period. In some embodiments, the six-month dosing cycle comprises administering 150 mg doses of the ADAMTS5 inhibiting polypeptide once a month for five consecutive months followed by a one-month resting period. In some embodiments, 150 mg doses of the ADAMTS5 inhibiting polypeptide are administered once a week for four, six, eight, ten, or twelve weeks with the remaining time in the six-month cycle a rest period. In some embodiments, 150 mg doses of the ADAMTS5 inhibiting polypeptide are administered at a frequency of once a week for four weeks and then once a month for four months followed a one-month resting period. In some embodiments, 150 mg doses of the ADAMTS5 inhibiting polypeptide are administered at a frequency of once a week for four weeks and then four 75 mg doses are administered once a month for four months followed by a one-month resting period.

In some embodiments, the ADAMTS5 inhibiting polypeptide is administered in a dosing regimen comprising one or more six-month dosing cycles. In this embodiment, 75 mg of the ADAMTS5 inhibiting polypeptide is administered once a month for three consecutive months followed by a three-month resting period. In some embodiments, the six-month dosing cycle comprises administering 75 mg doses of the ADAMTS5 inhibiting polypeptide once a month for five consecutive months followed by a one-month resting period. In some embodiments, 75 mg doses of the ADAMTS5 inhibiting polypeptide are administered once a week for four, six, eight, ten, or twelve weeks with the remaining time in the six-month cycle a rest period. In some embodiments, 75 mg doses of the ADAMTS5 inhibiting polypeptide are administered at a frequency of once a week for four weeks and then once a month for four months followed a one-month resting period. In some embodiments, 75 mg doses of the ADAMTS5 inhibiting polypeptide are administered at a frequency of once a week for four weeks and then four 50 mg doses are administered once a month for four months followed by a one-month resting period.

The timing of dosing in the dosing regimen is generally measured from the day of the first dose of the ADAMTS5 inhibiting polypeptide. However, different health care providers use different naming conventions. Notably, week zero may be referred to as week 1 by some health care providers, while day zero may be referred to as day one by some health care providers. Thus, it is possible that different physicians will designate, e.g., a dose as being given during week 3/on day 21, during week 3/on day 22, during week 4/on day 21, during week 4/on day 22, while referring to the same dosing schedule. For consistency, the first week of dosing will be referred to herein as week 1, while the first day of dosing will be referred to as day 1. However, it will be understood by a skilled artisan that this naming convention is simply used for consistency and should not be construed as limiting, i.e., weekly dosing is the provision of a weekly dose of the ADAMTS5 inhibiting polypeptide regardless of whether the physician refers to a particular week as “week 1” or “week 2”. It will further be understood that a dose need not be provided at an exact time point, e.g., a dose due approximately on day 29 could be provided, e.g., on day 24 to day 34, e.g., day 30, as long as it is provided in the appropriate week. Moreover, a “monthly dose” can be provided four to five weeks after the preceding dose.

In some embodiments, the methods may further comprise administration one or more doses of an effective amount of the ADAMTS5 inhibiting polypeptide. In some embodiments, the dose of the ADAMTS5 inhibiting polypeptide is about 50-1000 mg. In some embodiments, the dose of the ADAMTS5 inhibiting polypeptide is about 75-800 mg. In some embodiments, the dose of the ADAMTS5 inhibiting polypeptide is about 150-600 mg. In some embodiments, the dose of the ADAMTS5 inhibiting polypeptide is about 300-600 mg. In some embodiments, the dose of the ADAMTS5 inhibiting polypeptide is about 450-600 mg. In some embodiments, the dose of the ADAMTS5 inhibiting polypeptide is about 450-800 mg. In some embodiments, the dose of the ADAMTS5 inhibiting polypeptide is about 75-300 mg. In some embodiments, the dose of the ADAMTS5 inhibiting polypeptide is about 75-150 mg. In some embodiments, the dose of the ADAMTS5 inhibiting polypeptide is about 150-300 mg.

In some embodiments, the dose of the ADAMTS5 inhibiting polypeptide is about 75 mg. In some embodiments, the dose of the ADAMTS5 inhibiting polypeptide is about 150 mg. In some embodiments, the dose of the ADAMTS5 inhibiting polypeptide is about 300 mg. In some embodiments, the dose of the ADAMTS5 inhibiting polypeptide is about 450 mg. In some embodiments, the dose of the ADAMTS5 inhibiting polypeptide is about 600 mg. In some embodiments, the dose of the ADAMTS5 inhibiting polypeptide is about 750 mg. In some embodiments, the dose of the ADAMTS5 inhibiting polypeptide is about 800 mg. In some embodiments, the dose of the ADAMTS5 inhibiting polypeptide is about 1000 mg.

In some embodiments, the dose of the ADAMTS5 inhibiting polypeptide is about 300 mg. In some embodiments, the dose of the ADAMTS5 inhibiting polypeptide is about 150 mg. In some embodiments, the dose of the ADAMTS5 inhibiting polypeptide is about 75 mg.

In some embodiments, the dose of the ADAMTS5 inhibiting polypeptide is 50-1000 mg. In some embodiments, the dose of the ADAMTS5 inhibiting polypeptide is 75-800 mg. In some embodiments, the dose of the ADAMTS5 inhibiting polypeptide is 150-600 mg. In some embodiments, the dose of the ADAMTS5 inhibiting polypeptide is 300-600 mg. In some embodiments, the dose of the ADAMTS5 inhibiting polypeptide is 450-600 mg. In some embodiments, the dose of the ADAMTS5 inhibiting polypeptide is 450-800 mg. In some embodiments, the dose of the ADAMTS5 inhibiting polypeptide is 75-300 mg. In some embodiments, the dose of the ADAMTS5 inhibiting polypeptide is 75-150 mg. In some embodiments, the dose of the ADAMTS5 inhibiting polypeptide is 150-300 mg.

In some embodiments, the dose of the ADAMTS5 inhibiting polypeptide is 75 mg. In some embodiments, the dose of the ADAMTS5 inhibiting polypeptide is 150 mg. In some embodiments, the dose of the ADAMTS5 inhibiting polypeptide is 300 mg. In some embodiments, the dose of the ADAMTS5 inhibiting polypeptide is 450 mg. In some embodiments, the dose of the ADAMTS5 inhibiting polypeptide is 600 mg. In some embodiments, the dose of the ADAMTS5 inhibiting polypeptide is 750 mg. In some embodiments, the dose of the ADAMTS5 inhibiting polypeptide is 800 mg. In some embodiments, the dose of the ADAMTS5 inhibiting polypeptide is 1000 mg.

In some embodiments, the dose of the ADAMTS5 inhibiting polypeptide is 300 mg. In some embodiments, the dose of the ADAMTS5 inhibiting polypeptide is 150 mg. In some embodiments, the dose of the ADAMTS5 inhibiting polypeptide is 75 mg.

### EXAMPLES

The following examples are intended to illustrate various embodiments of the disclosure and are not to be construed as being limitations thereon. Abbreviations are used as is conventional in the art.

### Example 1: A Single-Center, Phase I, Randomized, Double Blind, Placebo-Controlled, First-In-Human Trial to Assess the Safety, Tolerability, Immunogenicity, Pharmacokinetics, and Pharmacodynamics of Single Ascending Doses of Subcutaneous Injected Compound 1 (Anti-ADAMTS-5 Nanobody) in Healthy Male Subjects

Study Design: This was a Phase I, single-center, randomized, double-blind, placebo-controlled, first-in-human trial, to assess the safety, tolerability, immunogenicity, pharmacokinetics and pharmacodynamics of single ascending doses of subcutaneous Compound 1 (Comp 1), an ADAMTS5 inhibiting polypeptide, in healthy male subjects. The main criteria for eligibility were that volunteers be healthy, non-smoking males aged 18-55 years (inclusive), with a body mass index between (BMI) 18.5 and 29.9 kg/m².

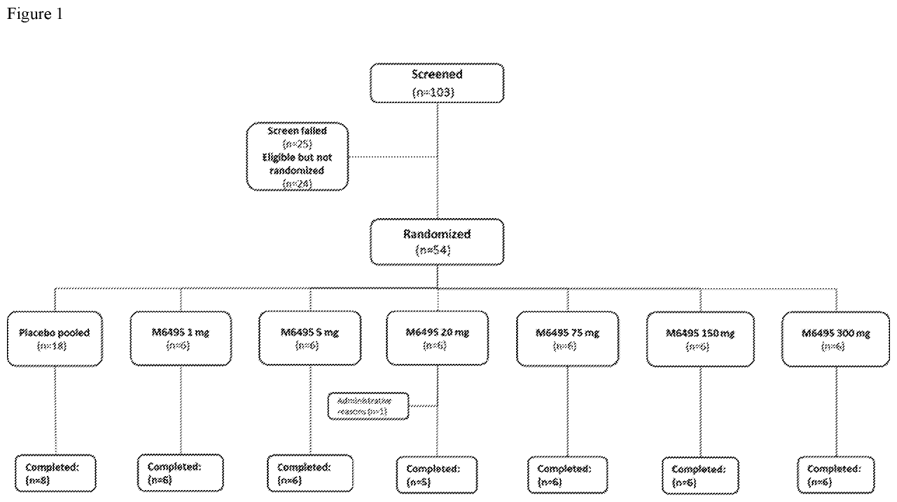

Subjects were randomized into 6 dose level cohorts (1 mg, 5 mg, 20 mg, 75 mg, 150 mg, 300 mg), of 9 subjects each. (FIG. 1). In each DL cohort, 9 healthy male participants received single doses of either Compound 1 or placebo in the ratio 6:3. The study included a 1-week in-house period after study intervention. After discharge, all subjects were to return for 7 ambulatory visits from Day 11 to Day 61 and a Follow up Visit on Day 75.

The primary study objective was to evaluate the safety and tolerability of single ascending doses of Compound 1 compared with placebo over 75 days. The safety endpoints were the nature, frequency, and severity of treatment-emergent adverse events (TEAEs) and serious adverse events (SAEs), local tolerability at the injection site, and ECG parameters. The secondary objectives included assessment of the pharmacokinetics of Compound 1, characterization and modelling of the PK/PD relationship between Compound 1 and ARGS, investigation of the proportional change from baseline in serum ARGS, assessment of the immunogenicity of Compound 1, and, as a safety evaluation, characterization of the slope of Compound 1 exposure versus corrected QT interval.

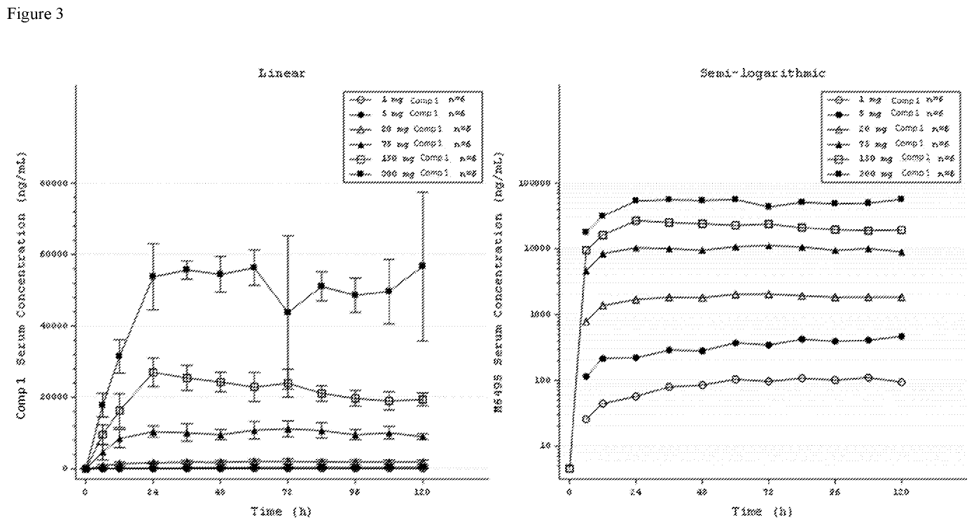

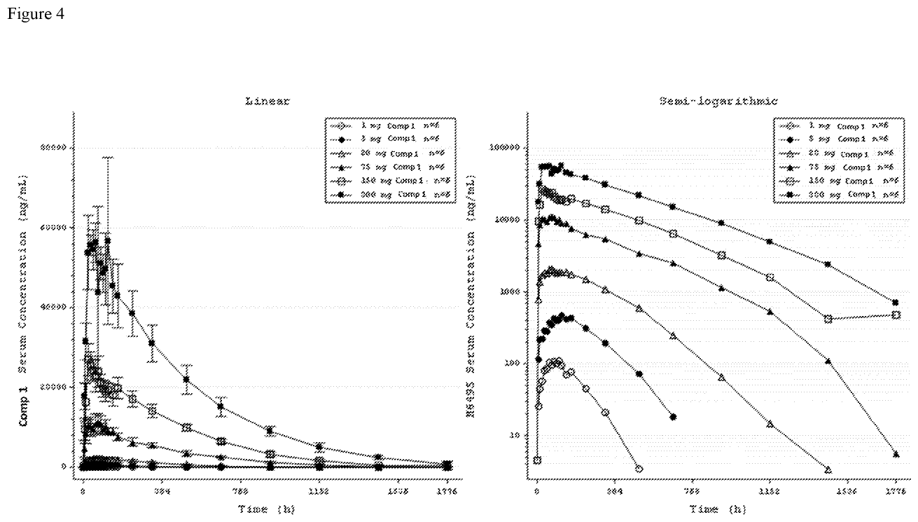

PK Results: Compound 1 was rapidly absorbed with detectable systemic levels at the earliest time point postdose (6 hours). Mean serum concentrations were then plateauing with levels relatively unchanged between approximately 24 and 120 hours. Then concentrations appeared to decline in a multiphasic manner and different slope according to the dose level (FIG. 3 and FIG. 4). Geometric mean AUCinf and Cmax increased greater than proportional with escalating doses from 1 mg to 300 mg. Similarly, observed terminal Tt1/2 was dose-dependent and increased supraproportionally with increasing dose.

Serum PK parameters and associated variabilities are presented below:

TABLE 6
Compound 1 Serum Key PK Parameters after
Single Dose in Healthy Male Participants

| | Geometric Mean (GCV %) | | | | | |
|---|---|---|---|---|---|---|
| | 1 mg | 5 mg | 20 mg | 75 mg | 150 mg | 300 mg |
| Parameter | n = 6 | n = 6 | n = 6 | n = 6 | n = 6 | n = 6 |
| AUCinf (μg · | 23.8 | 137 | 763 | 4550 | 11800 | 27900 |
| h/mL) | (10.9) | (36.2) | (31.6) | (11.7) | (9.4) | (12.6) |
| Cmax | 0.115 | 0.505 | 2.105 | 12.058 | 27.794 | 64.418 |
| (μg/mL) | (17.6) | (58.8) | (32.3) | (16.2) | (12.5) | (21.7) |
| Tmax (h)a | 90.12 | 120.04 | 72.12 | 66.08 | 24.06 | 36.09 |
| | (60.00- | (108.10- | (24.12- | (24.07- | (24.02- | (24.00- |
| | 108.08) | 168.05) | 168.07) | 120.00) | 60.02) | 119.90) |
| T½ (h) | 79.3 | 96.7 | 121 | 142 | 143 | 267 |
| | (22.6) | (16.7) | (40.5) | (30.1) | (49.5) | (15.9) |

GCV % = geometric coefficient of variation; n = number of subjects with available data in PK analysis.
aPresented as median (minimum-maximum).

Immunogenicity was assessed by measuring anti-drug antibodies (ADAs). Four subjects in the Compound 1 treatment groups (11%) and 2 subjects in the placebo group (11%) had a confirmed, positive ADA response. In 3 of 4 participants receiving Compound 1, ADA positivity was transient and in 1 participant all samples including the sample before dosing were positive. The presence of ADA did not appear to affect exposure to Compound 1.

PD Results: Aggrecan is a major structural proteoglycan found in the extracellular matrix of cartilage and it is depleted and shortened by the proteolytic activity of ADAMTS-5. ARGS is a neo-epitope formed during aggrecan degradation by ADAMTS-5 and its concentration in serum was used as the target engagement biomarker in nonclinical as well as clinical investigations. The ARGS analysis was performed in human serum samples using a partially validated, semi quantitative sandwich ELISA. In this assay, a biotinylated mAb that targets the G2 domain of aggrecan is coated on a streptavidin plate. ARGS-G2 present in serum binds to the antibody, and is detected with a monoclonal antibody specific to the ARGS neo-epitope linked to horse-radish peroxidase. ARGS concentrations were revealed by chemiluminescence reaction. The assay sensitivity was 55 pmol/L.

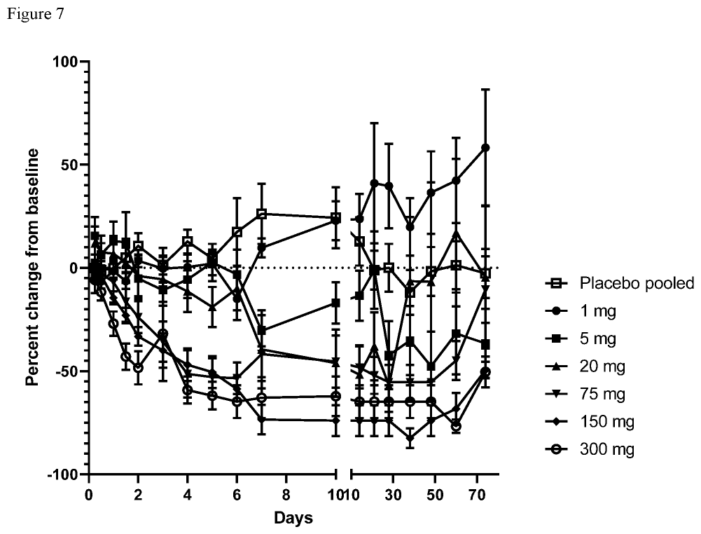

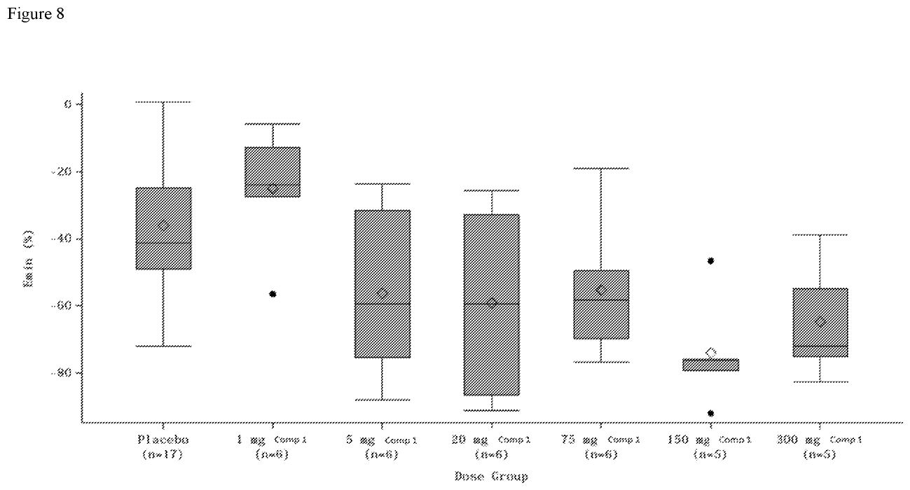

The ARGS response suggests target engagement at a systemic level and was in good agreement with the predictions based on the nonclinical data for the ARGS response in serum. Percent change from Baseline (CFB) and percent change from baseline profiles exhibited decreases in ARGS levels starting at 24 h with a clear differentiation at doses from 75 mg to 300 mg, and those at 20 mg and below by approximately 96 hours postdose. (FIG. 7). Mean percent serum concentrations were lower in the 75 mg, 150 mg, and 300 mg dose groups than in the placebo groups at all time-points. However, differentiation of a dose response between the 3 highest dose groups was limited by assay characteristics. In addition, suppression of ARGS level as exemplified by change- and percent change CFB were maintained below the Baseline in the 75 mg, 150 mg, and 300 mg dose groups through the end of the sampling period. A descriptive summary of each participant's maximum decrease in ARGS compared to Baseline (Emin=Imax, %) is shown in FIG. 8. The strongest mean maximum inhibition was observed with the 150 mg dose (74%).

Safety Results: Safety and tolerability of Compound 1 injections were assessed in healthy male participants, after single dose subcutaneous (sc) administrations of placebo or Compound 1 up to 300 mg. Overall, 36 participants were exposed to Compound 1, 6 in each dose group, and 18 received placebo (pooled analysis).

Compound 1 at single doses of up to 300 mg administered via sc injection was safe and well tolerated by the study population of healthy male subjects. No deaths, SAEs, or treatment-emergent adverse events (TEAEs) leading to discontinuation were reported during the study. Overall, 40 (74.1%) participants reported at least 1 TEAE; 12 (66.7%) participants on placebo and 28 (77.8%) participants on Compound 1.

The most prevalent TEAE were nasopharyngitis, headache, contact dermatitis, injection site reaction and myalgia. The majority of TEAEs were mild in severity. One participant, receiving 150 mg Compound 1, reported a severe TEAE of back pain (worsening of existing back pain), which was assessed as unrelated to Compound 1. The participant experienced episodes of back pain also prior to the period of this study. Six of the 36 participants receiving Compound 1 reported TEAEs that were considered related to Compound 1. These were injection site reactions, which were reported in 4 participants receiving Compound 1 (11.1% of all receiving Compound 1: 2 participants in the 75 mg dose cohort and 1 each in the 150 and 300 mg dose cohorts), and single incidences of abdominal pain, injection site pruritus, dizziness, and headache. Overall, there was minimal difference in the incidence of TEAEs between the Compound 1 DL cohorts and between treatment with placebo and Compound 1. No safety concerns based on laboratory measurements, vital signs, 12-lead ECG, or local tolerability were reported.

### Example 2: A Phase Ib, Single-Center, Double-Blind, Randomized, Placebo-Controlled, Parallel-Group, Multiple Ascending Dose Study to Assess Safety, Tolerability, Immunogenicity, Pharmacokinetics, and Pharmacodynamics of Subcutaneous Injections of Compound 1 (Anti-ADAMTS-5 Nanobody) in Participants with Symptomatic Knee Osteoarthritis

Study Design: This was a Phase Ib, single-center, randomized, double-blind, placebo-controlled, parallel-group, multiple ascending dose study, designed to assess safety, tolerability, immunogenicity, PK, and PD of subcutaneous Compound 1 in patients with symptomatic OA of the knee. The study was open to male or female participants, 40-75 years of age (inclusive) with KL radiological grade of 2-4 and Western Ontario McMaster Universities Osteoarthritis Index (WOMAC) pain sub scores of ≥40 out of 100 in the target knee. The knee X-ray was not to be older than 6 months and had to be assessed by a radiologist prior to randomization to verify the radiographic (KL) score.

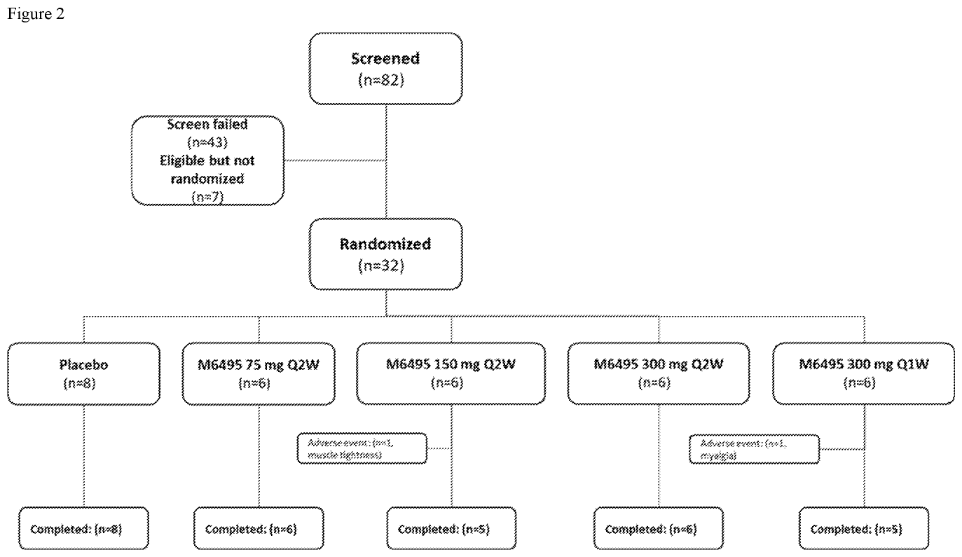

Patients were randomized into one of 3 dose level cohorts, of 75 mg, 150 mg, and 300 mg, to receive fortnightly subcutaneous Compound 1 or placebo (at a ratio of 6:2). (FIG. 2). As safety data from these cohorts and from the prior single ascending dose study did not suggest any safety concerns, a fourth cohort was formed, for weekly administration of 300 mg. The primary objective of the study was to evaluate the safety and tolerability of repeated doses of Compound 1 in participants with symptomatic knee OA over a period of 106 days. The secondary objectives were to evaluate the PK of Compound 1 and to characterize its immunogenicity. The exploratory objectives included evaluating the effects of Compound 1 on the inhibition of serum ARGS (and the PK/PD relationship), and the exposure-QT interaction expected tₘₐₓ.

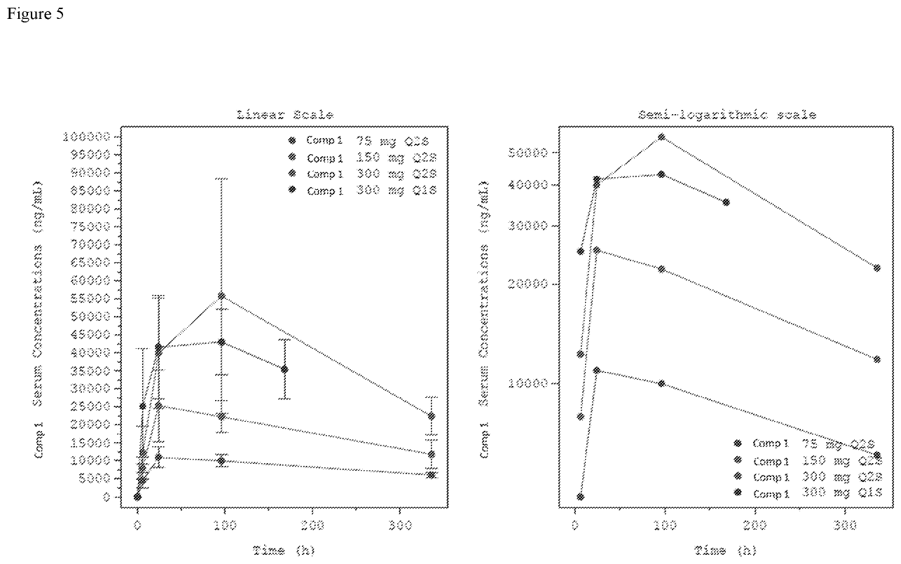

PK Results: Following a single sc injection on Day 1, serum concentrations (above LLOQ of 5.831 ng/mL) of Compound 1 were detected in the 75 mg, 150 mg, and 300 mg EOW and 300 mg weekly Compound 1 cohorts. Mean Compound 1 serum concentrations peaked at 24 hours postdose for the 75 mg and 150 mg EOW cohorts, and at 96 hours postdose for the 300 mg EOW and weekly cohorts. For each nominal time point, the mean Compound 1 concentrations increased with dose in the dose administrations EOW cohorts. Those results showed that Compound 1 was rapidly absorbed with detectable systemic level at the earliest sample time point postdose (6 hours) (FIG. 5).

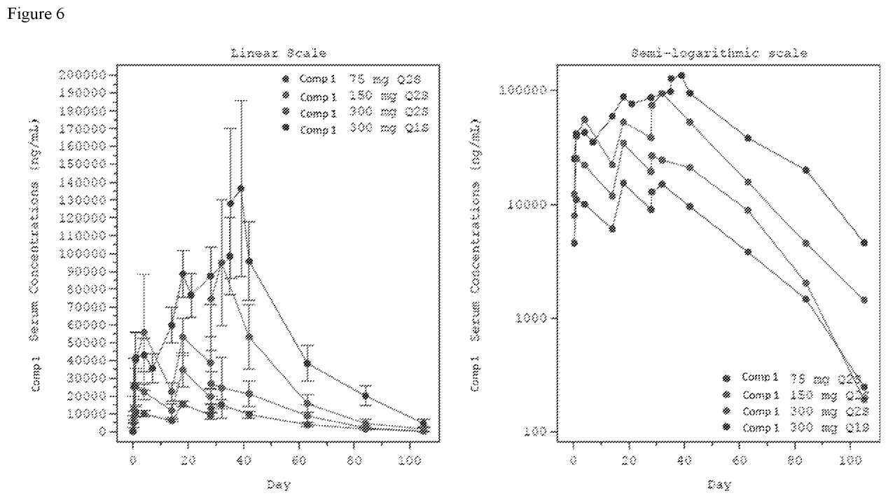

Following multiple sc injections on Day 29 for dose administration EOW and on Day 36 for weekly dose administration, mean Compound 1 serum concentrations peaked at 96 hours postdose for all cohorts except for the 150 mg EOW cohort where mean concentrations peaked at 6 hours postdose. In general, mean Compound 1 concentrations increased with the dose in EOW cohorts. Mean Compound 1 concentrations were highest on Day 36 at 96 hours postdose for 300 mg weekly (FIG. 6).

On Day 1, geometric mean AUCtau and Cmax showed an approximately dose-proportional increase with escalating doses from 75 mg to 300 mg EOW. Median Compound 1 Ttmax was approximately 24 hours in the 75 mg and 150 mg EOW cohorts, and approximately 97 hours in the 300 mg EOW and weekly cohorts:

TABLE 7
Compound 1 Serum Key Pharmacokinetic
Parameters for each Dose Level on Day 1

| | Geometric Mean (GeoCV %) | | | |
|---|---|---|---|---|
| | Comp. 1 | Comp. 1 | Comp. 1 | Comp. 1 |
| | 75 mg | 150 mg | 300 mg | 300 mg |
| Para- | EOW | EOW | EOW | Weekly |
| meter | n = 6 | n = 6 | n = 6 | n = 6 |
| AUCtau | 2.79 (16.9) | 6.77 (4.8) ^{b} | 12.2 (29.1) | 6.19 (27.4) ^{c} |
| (mg · | | | | |
| h/mL) | | | | |
| AUC | 0.0372 (16.9) | 0.0451 (4.8) ^{b} | 0.0406 (29.1) | 0.0206 (27.4) ^{c} |
| tau/Dose | | | | |
| (mg · h/ | | | | |
| mL/mg) | | | | |
| Cmax | 10900 (24.8) | 27200 (30.4) | 54600 (51.3) | 44500 (30.4) |
| (ng/ml) | | | | |
| Cmax/ | 146 (24.8) | 181 (30.4) | 182 (51.3) | 148 (30.4) |
| Dose | | | | |
| (ng/ml/ | | | | |
| mg) | | | | |
| Imax | 24.06 | 24.25 | 97.60 | 97.27 |
| (h) ^{a} | (23.25-98.50) | (23.62-98.05) | (23.88-126.62) | (23.87-98.62) |

GeoCV % = geometric coefficient of variation,
EOW = every other week.
^{a} Presented as median (minimum-maximum)
^{b} n = 3
^{c} n = 4
tau = 336 hours in the EOW regimens and 168 hours in the weekly regimen.

Following multiple dosing in the 300 mg weekly cohort, Compound 1 exposure doubled in the first 2 weeks with an Racc (Cmax) value approximating 2. On Day 29, median Compound 1 Ttmax ranged from approximately 95 hours to 109 hours in the EOW dose administration cohorts compared to approximately 119 hours in the 300 mg weekly cohort. Accumulation of AUCtau and Cmax was generally within 2-fold across the dose administration EOW cohorts while it was approximately 3-fold in the 300 mg weekly cohort:

TABLE 8
Compound 1 Serum Key Pharmacokinetic Parameters for each
Dose Level on Day 29 (Day 36 for 300 mg weekly)

| | Geometric Mean (GeoCV %) | | | |
|---|---|---|---|---|
| | Comp. 1 | Comp. 1 | Comp. 1 | Comp. 1 |
| | 75 mg | 150 mg | 300 mg | 300 mg |
| Para- | EOW | EOW | EOW | Weekly |
| meter | n = 6 | n = 5 | n = 5 | n = 5 |
| AUCtau | 4.20 (16.4) | 7.79 (66.7) | 24.1 (31.8) | 20.1 (37.7) |
| (mg · | | | | |
| h/mL) | | | | |
| AUCtau/ | 0.0560 (16.4) | 0.0519 (66.7) | 0.0804 (31.8) | 0.0671 (37.7) |
| Dose | | | | |
| (mg · h/ | | | | |
| mL/mg) | | | | |
| Cmax | 15100 (14.8) | 35600 (52.7) | 93900 (35.1) | 136000 (43.1) |
| (ng/mL) | | | | |
| Cmax/ | 202 (14.8) | 238 (52.7) | 313 (35.1) | 454 (43.1) |
| Dose | | | | |
| (ng/ml/ | | | | |
| mg) | | | | |
| Tmax | 109.20 | 95.95 | 95.45 | 119.33 |
| (h) ^{a} | (6.02-124.33) | (5.92-220.68) | (95.18-311.17) | (5.80-124.75) |
| Ctrough | 8870 (21.5) | 19100 (20.9) | 36800 (35.4) | 96500 (24.0) |
| (ng/ml) | | | | |
| Ctrough/ | 118 (21.5) | 128 (20.9) | 123 (35.4) | 322 (24.0) |
| Dose | | | | |
| (ng/ml/ | | | | |
| mg) | | | | |
| Racc | 1.39 (16.8) | 1.32 (28.1) | 1.63 (76.5) | 3.06 (21.8) |
| (Cmax) | | | | |
| Racc | 1.51 (11.6) | 1.46 (69.8) | 1.95 (49.6) | 3.12 (17.6) ^{b} |
| (AUCtau) | | | | |

GeoCV % = geometric coefficient of variation,
EOW = every other week.
^{a} Presented as median (minimum-maximum)
^{b} n = 3
tau = 336 hours in the EOW regimens and 168 hours in the weekly regimen.

PK parameters from the EOW and the weekly cohorts are only comparable to a limited extent, due to the different dosing regimens. Apparently, steady state had not been reached in the EOW and weekly cohorts based on visual inspection of the PK concentration versus time plots. Assessment of dose proportionality was conducted using data from the EOW regimens only. Overall, AUCtau and Cmax appeared to increase in an approximately dose-proportional manner across the dose range of 75 mg to 300 mg.

One participant in the 150 mg EOW cohort, 1 participant in the 300 mg EOW cohort, and 3 participants in the 300 mg weekly cohort had confirmed positive ADA test results. The participant in the 150 mg EOW cohort had preexisting ADAs, which were not boosted during treatment. In summary, a total of 4 out of 24 participants had treatment-emergent ADAs. Positive treatment-emergent ADA test results were observed starting at Day 64 (N=2) and Day 85 (N=2). AUCtau data were visually compared between ADA negative and positive results. Except for 1 participant in the 300 mg weekly dose level cohort without detectable ADA, AUCtau observed in participants with ADA positive results were comparable to AUCtau in participants with ADA negative results. Over the entire sampling period the Compound 1 exposure of this 1 ADA positive participant in the 300 mg weekly dose level cohort was lower than for other participants in the same dose cohort. There was no clear temporal relationship between ADA formation (Day 64 and Day 85) and changes in Compound 1 exposure levels.

PD Results: ARGS was measured as a target engagement and PD biomarker with the same method as described in Example 1. The LLOQ of the assay in this study was 80 pmol/L.

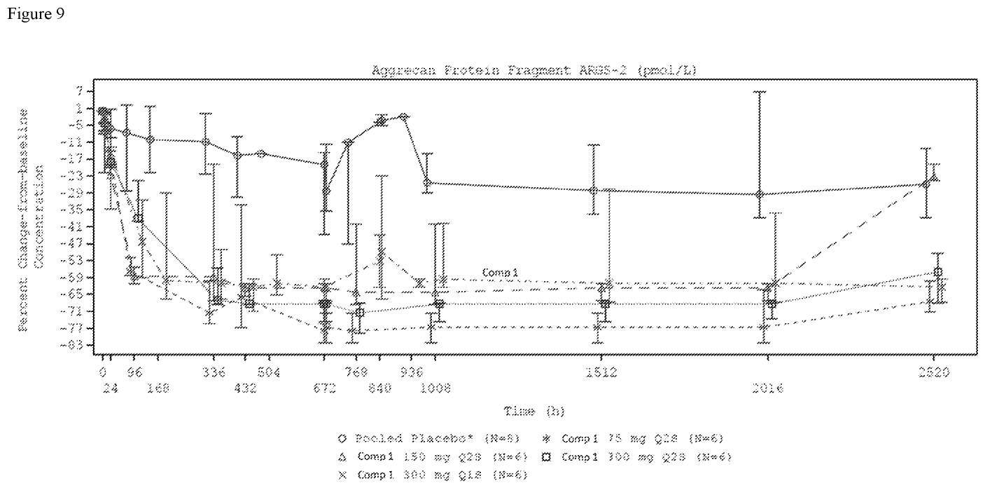

Mean change from baseline (CFB) and percent CFB exhibited decreases in ARGS levels starting at 6 hours (FIG. 9). Numerically, mean CFB serum concentrations were generally lower in all 4 dose levels than in the placebo group at most of the time-points: target engagement was achieved (≥35% mean CFB compared to placebo). The least square mean of percent CFB (95% CI) for Compound 1 75 mg EOW at end of treatment was −76.8% (−90.7-62.8%). An approximately 50% mean decrease (versus placebo) in ARGS concentration was observed from Day 15 and maintained through the EOS (Day 106). However, differentiation of dose response among the 4 treatments was not apparent.

Safety Results: Multiple sc doses of Compound 1 up to 300 mg weekly were generally considered well tolerated and safe for the population evaluated. Overall, there was minimal difference in the incidence of TEAEs between the different Compound 1 cohorts and between treatment with Compound 1 and placebo, however the incidence of treatment-related TEAEs were higher in the higher dose cohorts.

No deaths were observed. Two patients presented serious TEAEs (grade 3). One patient in cohort 2 (150 mg EOW) reported asthma (grade 3) after the second dose, but the event was considered to be provoked by a flu and not to be related to Compound 1 by the investigator. The event resolved with no recurrence and the patient received the third dose without further complications. The second participant was allocated to placebo and registered increase in knee OA pain (grade 3) that was not considered to be related to the study treatment, required no change to the treatment and resolved before the EOS.

Two participants were discontinued from the study due to musculoskeletal TEAEs. One participant from dose cohort 2 (150 mg EOW) experienced muscle tightness grade 1 and was withdrawn from the study before receiving their third dose. The event was mild, did not prevent any daily activities, and was considered as unrelated to the study treatment. The outcome was recorded as not recovered/not resolved as the participant was lost to follow-up. The second participant, who was allocated to cohort 4 (300 mg weekly), reported myalgia grade 1 in the upper arms, buttocks, thigh, upper back and calves on Days 17, 29 and 36 and was permanently discontinued from the study but attended the follow-up visits. The patient received treatment for pain management and the AEs resolved at the time of discontinuation. The events were not considered treatment related.

Ten patients reported TEAEs that were assessed as related to the study treatment. The most common TEAEs reported for all treatments were headache, arthralgia, injection site reaction and muscle tightness. All reported injection site reactions were grade 1 in severity and resolved before the EOS. Two participants received topical treatment and the other reported events resolved without intervention. Injection site pain was reported by 1 participant receiving 300 mg EOW. The TEAE resolved before the EOS with no intervention. There was no apparent correlation between the formation of ADA and the incidence of injection site reactions.

The incidence of muscle-related events was higher in subjects receiving Compound 1 compared to placebo and severity was higher in patients in the 300 mg EOW and 300 mg weekly cohorts. None of these events were associated with any relevant creatine phosphokinase (CPK) increase.

### Example 3: A Randomized, Two-Arm, Placebo-Controlled, Participant, Investigator and Sponsor-Blinded, Proof-of-Concept Study Investigating the Efficacy, Safety and Tolerability of Compound 1 (Anti-ADAMTS-5 Nanobody) in Patients with Symptomatic Knee Osteoarthritis

This is a proof-of-concept (POC) study to establish the effect of Compound 1 on pain and cartilage preservation, in patients with symptomatic knee OA. While the benefits on pain and structure of the inhibition of ADAMTS-5 have been demonstrated in pre-clinical studies, its role as a therapeutic target for patients with OA is still to be defined. The effect of Compound 1 on pain in the target knee of patients with symptomatic knee OA will be evaluated after 12 weeks of treatment, and the effect on preservation of cartilage volume in the target knee after 52 weeks of treatment.

Study Design—This is a non-confirmatory study using a randomized, two-treatment arm, parallel-group, participant, investigator and sponsor-blinded, placebo-controlled design, with the purpose of investigating the efficacy, safety and tolerability of s.c. injections of Compound 1 300 mg vs placebo every 4 weeks (q4w), in approximately 98 patients with symptomatic knee OA. The study consists of a screening period of up to approximately 6 weeks, used to assess eligibility and to taper participants off prohibited medications. Between Day −4 to −1 visit, eligible participants will be randomized to one of the treatment arms in a ratio of 1:1. On Day 1 randomized participants will enter the treatment period, during which they will receive q4w sc injections of Compound 1 300 mg or placebo. The last study treatment administration will occur at Week 48 and the end of treatment (EOT) visit will occur 4 weeks later, at Week 52. An end of study (EOS) visit will occur 8 weeks after the EOT visit, at Week 60. The total study duration from screening to EOS is expected to be a maximum of 66 weeks. The primary analysis will be performed when all participants have reached Week 12, in order to assess the primary endpoint (KOOS pain at Week 12).

The screening period will consist of three visits:
Screening 1—Following the signing of the informed consent, participants will undergo assessments to confirm their eligibility. The required assessments may be conducted over several days if it is in the best interest of the participants, or for logistical reasons. X-ray will be performed in the target knee and sent for central assessment by an independent reviewer, to assess participant's eligibility. Participants treated with prohibited concomitant medications will initiate a washout period at Screening 1. Screening 1 may be scheduled up to 40 calendar days prior to Day 1 but should ideally occur as close as possible prior to Day 1.
Screening 2—It should be scheduled between 20 and 9 calendar days prior to Day 1. Participants will go to the clinical site for pain assessment and review of the results of the Screening 1 assessments. If the participants are still eligible, they will be instructed to complete a pain diary every day from this visit until the Week 12 visit, and to wear an actigraphy sensor during daytime from this visit until the Week 4 visit.

Screening 3 (phone call)—Eligibility will be re-assessed based on the results of the pain diary and KOOS pain of 7 days prior to the phone call. The investigator may schedule the call between Day −4 and Day −1, allowing the participant to complete at least 7 days of the pain diary and the KOOS pain questionnaire. If eligibility is confirmed, the participant will be randomized to one of the treatment arms.

During the treatment period, participants will undergo efficacy, safety, PK and PD assessments. The treatment period will consist of 16 visits:
Treatment initiation visit on Day 1: first study treatment administration.
Participants will have on-site assessments on Day 1, Day 5, Week 4, Week 8, and Week 12, as outlined in the assessment schedule.
After Week 12, participants may have the possibility to perform some visits in an off-site setting if allowed by local legislation and if feasible based on local capabilities:
   On-site visits: Week 28 (including a knee MRI) and Week 40;
   On-site or off-site visits (as applicable): Weeks 16, 20, 24, 32, 36, 44, 48. Off-site visits will include s.c. administration of QUC398 300 mg or placebo, as well as pain assessment with PROs and blood/urine sampling according to the Assessment Schedule.
An on-site EOT visit will occur at Week 52, i.e. 4 weeks after the last study treatment administration, and will include a knee MRI.

Participants will be followed up for 8 weeks after the EOT visit. An optional visit will occur at week 56, during which blood samples will be taken for PK and PD assessment. Participants may have the possibility to perform this visit in a remote setting, if allowed by local legislation and feasible based on local capabilities. An on-site EOS visit will then occur at Week 60, i.e., 8 weeks after the EOT visit.

Study Population—The study population consists of male and female adult patients between 40 and 80 years of age with symptomatic, mild to severe radiographic knee OA (Kellgren-Lawrence [KL] grade 2-4). Approximately 98 participants are planned to be randomized in this study. The investigator must ensure that a patient meets all of the inclusion and none of the exclusion criteria before enrolling him/her into the trial. No additional criteria should be applied by the investigator when considering a patient's eligibility. In the case where a safety laboratory assessment at screening meets any of the exclusion criteria but the condition is expected to be transient and resolved prior to first dose, the assessment may be repeated once prior to randomization. If the repeat value remains exclusionary, then the participant will be excluded from the study.

Results—The primary endpoint to be evaluated is efficacy of Compound 1 at relieving OA pain in the target knee at Week 12. Pain relief will be measured by the change from baseline in KOOS pain subscale at Week 12. KOOS pain subscale score will be analyzed on 100 points scale, a low score meaning high pain. It is anticipated that treatment with Compound 1 will result in a decrease in OA pain in the target knee as compared to placebo.

The study will also assess the efficacy of Compound 1 compared to placebo in meeting several secondary objectives:
The efficacy of Compound 1 at relieving OA pain in the target knee over time. Pain relief will be measured by the change from baseline in KOOS pain subscale and the change from baseline in Pain Numerical Rating Scale (NRS). KOOS pain subscale score will be analyzed on 100 points scale, a low score meaning high pain. It is anticipated that treatment with Compound 1 will result in a decrease in OA pain in the target knee over time as compared to placebo.
The efficacy of Compound 1 at preserving cartilage in the medial compartment of the target knee as compared to placebo. MRI will be used to monitory the effects of Compound 1 on cartilage structure and composition. MRI will be used to quantify changes in volume and thickness of cartilage; furthermore changes in cartilage quality based on its T2 relaxation time and a so-called Radiomics approach, a quantitative method that extracts a large number of features from T2 maps using data-characterization algorithms to inform on cartilage texture will be assessed. MRI will be taken at Day 1, Week 28 and Week 52. MRI can be performed before or after other clinical assessments at a visit, also on a different day if needed for logistical reasons, but should always be done within the visit window. It is anticipated that treatment with Compound 1 will result in a preservation of cartilage in the target knee as compared to placebo.
The efficacy of Compound 1 at relieving clinical symptoms of OA and improving function in the target knee as compared to placebo. Relief of clinical symptoms of OA and improvement in function will be measured by the change in total KOOS score, change in KOOS subscale, and the change from baseline in Patient's Global Assessment (PGA) as assessed by NRS. The 30 second chair stand test and 2 minute walk test are standardized performance outcome measurements will also be used to evaluate the change in function throughout and at completion of the treatment period. It is anticipated that treatment with Compound 1 will result in relief of clinical symptoms and an improved function in the target knee as compared to placebo.

### Numbered Embodiments

Notwithstanding the appended claims, the following numbered embodiments are also contemplated herein and form part of the instant disclosure.
1. A method of treating osteoarthritis in a subject in need thereof comprising administering 75-300 mg of an ADAMTS5 inhibiting polypeptide to a human subject.
2. The method of embodiment 1, wherein the ADAMTS5 inhibiting polypeptide comprises at least one immunoglobulin single variable domain (ISVD) comprising 3 complementarity determining regions (CDR), wherein the complementary determining regions are CDR1 to CDR3, in which (i) CDR1 is selected from the group consisting of SEQ ID NOs: 21, 35, 20, 22, 25, 33, 28, 24, 23, 26, 27, 29, 30, 31, 32 and 34; (ii) CDR2 is selected from the group consisting of SEQ ID NOs: 37, 53, 36, 40, 50, 51, 44, 45, 43, 39, 38, 41, 119, 42, 46, 47, 48, 49 and 52; and (iii) CDR3 is selected from the group consisting of SEQ ID NOs: 55, 118, 71, 54, 58, 68, 69, 62, 63, 61, 57, 56, 59, 60, 64, 65, 66, 67 and 70.
3. The method of embodiment 2, wherein the ADAMTS5 inhibiting polypeptide comprises at least one ISVD comprising 3 CDRs, wherein the CDRs are CDR1 to CDR3, in which:
   (i) CDR1 is SEQ ID NO: 21, CDR2 is SEQ ID NO: 37 and CDR3 is SEQ ID NO: 55;
   (ii) CDR1 is SEQ ID NO: 35, CDR2 is SEQ ID NO: 53 and CDR3 is SEQ ID NO: 118;
   (iii) CDR1 is SEQ ID NO: 35, CDR2 is SEQ ID NO: 53 and CDR3 is SEQ ID NO: 71;
   (iv) CDR1 is SEQ ID NO: 20, CDR2 is SEQ ID NO: 36 and CDR3 is SEQ ID NO: 54;
   (v) CDR1 is SEQ ID NO: 22, CDR2 is SEQ ID NO: 36 and CDR3 is SEQ ID NO: 54;
   (vi) CDR1 is SEQ ID NO: 25, CDR2 is SEQ ID NO: 40 and CDR3 is SEQ ID NO: 58;
   (vii) CDR1 is SEQ ID NO: 33, CDR2 is SEQ ID NO: 50 and CDR3 is SEQ ID NO: 68;
   (viii) CDR1 is SEQ ID NO: 33, CDR2 is SEQ ID NO: 51 and CDR3 is SEQ ID NO: 69;
   (ix) CDR1 is SEQ ID NO: 28, CDR2 is SEQ ID NO: 44 and CDR3 is SEQ ID NO: 62;
   (x) CDR1 is SEQ ID NO: 28, CDR2 is SEQ ID NO: 45 and CDR3 is SEQ ID NO: 63;
   (xi) CDR1 is SEQ ID NO: 28, CDR2 is SEQ ID NO: 43 and CDR3 is SEQ ID NO: 61;
   (xii) CDR1 is SEQ ID NO: 24, CDR2 is SEQ ID NO: 39 and CDR3 is SEQ ID NO: 57;
   (xiii) CDR1 is SEQ ID NO: 23, CDR2 is SEQ ID NO: 38 and CDR3 is SEQ ID NO: 56;
   (xiv) CDR1 is SEQ ID NO: 26, CDR2 is SEQ ID NO: 41 and CDR3 is SEQ ID NO: 59;
   (xv) CDR1 is SEQ ID NO: 27, CDR2 is SEQ ID NO: 119 and CDR3 is SEQ ID NO: 60;
   (xvi) CDR1 is SEQ ID NO: 27, CDR2 is SEQ ID NO: 42 and CDR3 is SEQ ID NO: 60;
   (xvii) CDR1 is SEQ ID NO: 29, CDR2 is SEQ ID NO: 46 and CDR3 is SEQ ID NO: 64;
   (xviii) CDR1 is SEQ ID NO: 30, CDR2 is SEQ ID NO: 47 and CDR3 is SEQ ID NO: 65;
   (xix) CDR1 is SEQ ID NO: 31, CDR2 is SEQ ID NO: 48 and CDR3 is SEQ ID NO: 66;
   (xx) CDR1 is SEQ ID NO: 32, CDR2 is SEQ ID NO: 49 and CDR3 is SEQ ID NO: 67; or
   (xxi) CDR1 is SEQ ID NO: 34, CDR2 is SEQ ID NO: 52 and CDR3 is SEQ ID NO: 70.
4. The method of embodiment 3, wherein the ADAMTS5 inhibiting polypeptide comprises at least one ISVD comprising 3 CDRs, wherein the CDRs are CDR1 to CDR3, in which CDR1 is SEQ ID NO: 21, CDR2 is SEQ ID NO: 37 and CDR3 is SEQ ID NO: 55.
5. The method of embodiment 1, wherein the ISVD is chosen from the group consisting of SEQ ID NOs: 2, 116, 19, 1, 3, 6, 16, 17, 10, 11, 9, 5, 4, 7, 8, 117, 12, 13, 14, 15, 18, and amino acids 1-124 of SEQ ID NO: 129.
6. The method of embodiment 5, wherein the ISVD is amino acids 1-124 of SEQ ID NO: 129.
7. The method of embodiment 1, wherein the ADAMTS5 inhibiting polypeptide comprises two or more ISVDS, wherein a) at least a first ISVD specifically binds a first antigenic determinant, epitope, part, domain, subunit or conformation of ADAMTS5; and wherein, b) at least a second ISVD specifically binds a second antigenic determinant, epitope, part, domain, subunit or conformation of serum albumin.
8. The method of embodiment 7, wherein the first ISVD is chosen from the group consisting of SEQ ID NOs: 2, 116, 19, 1, 3, 6, 16, 17, 10, 11, 9, 5, 4, 7, 8, 117, 12, 13, 14, 15, 18, and amino acids 1-124 of SEQ ID NO: 129.
9. The method of embodiment 8, wherein the first ISVD is amino acids 1-124 of SEQ ID NO: 129.
10. The method of embodiment 7, wherein the second ISVD is chosen from the group consisting of SEQ ID NOs: 131, 132, 133, 134, 135, 136, 137, 138, 139, 140, 141, 142, 143, 144, or 145.
11. The method of embodiment 10, wherein the second ISVD is SEQ ID NO: 138.
12. The method of embodiment 7, wherein the first ISVD is amino acids 1-124 of SEQ ID NO: 129, and the second ISVD is SEQ ID NO: 138.
13. The method of embodiment 1, wherein the ADAMTS5 inhibiting polypeptide is selected from the group consisting of SEQ ID NO: 129 (clone 577 2F3^{so}-Alb), SEQ ID NO: 130 (clone 579 2F3^{so}-093-Alb), SEQ ID NO: 120 (clone 4 2A12-Alb), SEQ ID NO: 121 (clone 5 2D7-Alb), SEQ ID NO: 122 (clone 6 2F3-Alb), SEQ ID NO: 123 (clone 69 049-Alb), SEQ ID NO: 124 (clone 70 9D3-Alb), SEQ ID NO: 125 (clone 71 3B2-Alb), SEQ ID NO: 126 (clone 129 2F3-093-Alb), SEQ ID NO: 127 (clone 130 049-093-Alb), and SEQ ID NO: 128 (clone 131 9D3-093-Alb).
14. The method of embodiment 13, wherein the ADAMTS5 inhibiting polypeptide is SEQ ID NO: 129 (clone 577 2F3^{so}-Alb).
15. The method of any of embodiments 1-14, wherein the osteoarthritis is knee osteoarthritis.
16. The method of any of embodiments 1-15, wherein the dose of the ADAMTS5 inhibiting polypeptide is 150 mg.
17. The method of any of embodiments 1-15, wherein the dose of the ADAMTS5 inhibiting polypeptide is 300 mg.
18. The method of any of embodiments 1-17, wherein the ADAMTS5 inhibiting polypeptide is administered once a month.
19. The method of any of embodiments 1-18, wherein administration of the ADAMTS5 inhibiting polypeptide decreases the formation of ARGS fragments.
20. The method of any of embodiments 1-19, wherein administration of the ADAMTS5 inhibiting polypeptide decreases pain in an osteoarthritis affected joint as determined by KOOS score or WOMAC score.
21. The method of any of embodiments 1-20, wherein administration of the ADAMTS5 inhibiting polypeptide maintains the structure of an osteoarthritis affected joint as determined by automated segmentation in MRI.

What is claimed is:

1. A method of treating osteoarthritis in a subject in need thereof comprising administering 75-300 mg of an ADAMTS5 inhibiting polypeptide to a human subject.

2. The method of claim 1, wherein the ADAMTS5 inhibiting polypeptide comprises at least one immunoglobulin single variable domain (ISVD) comprising 3 complementarity determining regions (CDR), wherein the complementary determining regions are CDR1 to CDR3, in which
(i) CDR1 is selected from the group consisting of SEQ ID NOs: 21, 35, 20, 22, 25, 33, 28, 24, 23, 26, 27, 29, 30, 31, 32 and 34;
(ii) CDR2 is selected from the group consisting of SEQ ID NOs: 37, 53, 36, 40, 50, 51, 44, 45, 43, 39, 38, 41, 119, 42, 46, 47, 48, 49 and 52; and
(iii) CDR3 is selected from the group consisting of SEQ ID NOs: 55, 118, 71, 54, 58, 68, 69, 62, 63, 61, 57, 56, 59, 60, 64, 65, 66, 67 and 70.

3. The method of claim 2, wherein the ADAMTS5 inhibiting polypeptide comprises at least one ISVD comprising 3 CDRs, wherein the CDRs are CDR1 to CDR3, in which:
(i) CDR1 is SEQ ID NO: 21, CDR2 is SEQ ID NO: 37 and CDR3 is SEQ ID NO: 55;
(ii) CDR1 is SEQ ID NO: 35, CDR2 is SEQ ID NO: 53 and CDR3 is SEQ ID NO: 118;
(iii) CDR1 is SEQ ID NO: 35, CDR2 is SEQ ID NO: 53 and CDR3 is SEQ ID NO: 71;
(iv) CDR1 is SEQ ID NO: 20, CDR2 is SEQ ID NO: 36 and CDR3 is SEQ ID NO: 54;
(v) CDR1 is SEQ ID NO: 22, CDR2 is SEQ ID NO: 36 and CDR3 is SEQ ID NO: 54;
(vi) CDR1 is SEQ ID NO: 25, CDR2 is SEQ ID NO: 40 and CDR3 is SEQ ID NO: 58;
(vii) CDR1 is SEQ ID NO: 33, CDR2 is SEQ ID NO: 50 and CDR3 is SEQ ID NO: 68;
(viii) CDR1 is SEQ ID NO: 33, CDR2 is SEQ ID NO: 51 and CDR3 is SEQ ID NO: 69;
(ix) CDR1 is SEQ ID NO: 28, CDR2 is SEQ ID NO: 44 and CDR3 is SEQ ID NO: 62;
(x) CDR1 is SEQ ID NO: 28, CDR2 is SEQ ID NO: 45 and CDR3 is SEQ ID NO: 63;
(xi) CDR1 is SEQ ID NO: 28, CDR2 is SEQ ID NO: 43 and CDR3 is SEQ ID NO: 61;
(xii) CDR1 is SEQ ID NO: 24, CDR2 is SEQ ID NO: 39 and CDR3 is SEQ ID NO: 57;
(xiii) CDR1 is SEQ ID NO: 23, CDR2 is SEQ ID NO: 38 and CDR3 is SEQ ID NO: 56;
(xiv) CDR1 is SEQ ID NO: 26, CDR2 is SEQ ID NO: 41 and CDR3 is SEQ ID NO: 59;
(xv) CDR1 is SEQ ID NO: 27, CDR2 is SEQ ID NO: 119 and CDR3 is SEQ ID NO: 60;
(xvi) CDR1 is SEQ ID NO: 27, CDR2 is SEQ ID NO: 42 and CDR3 is SEQ ID NO: 60;
(xvii) CDR1 is SEQ ID NO: 29, CDR2 is SEQ ID NO: 46 and CDR3 is SEQ ID NO: 64;
(xviii) CDR1 is SEQ ID NO: 30, CDR2 is SEQ ID NO: 47 and CDR3 is SEQ ID NO: 65;
(xix) CDR1 is SEQ ID NO: 31, CDR2 is SEQ ID NO: 48 and CDR3 is SEQ ID NO: 66;
(xx) CDR1 is SEQ ID NO: 32, CDR2 is SEQ ID NO: 49 and CDR3 is SEQ ID NO: 67; or
(xxi) CDR1 is SEQ ID NO: 34, CDR2 is SEQ ID NO: 52 and CDR3 is SEQ ID NO: 70.

4. The method of claim 3, wherein the ADAMTS5 inhibiting polypeptide comprises at least one ISVD comprising 3 CDRs, wherein the CDRs are CDR1 to CDR3, in which CDR1 is SEQ ID NO: 21, CDR2 is SEQ ID NO: 37 and CDR3 is SEQ ID NO: 55.

5. The method of claim 1, wherein the ISVD is chosen from the group consisting of SEQ ID NOs: 2, 116, 19, 1, 3, 6, 16, 17, 10, 11, 9, 5, 4, 7, 8, 117, 12, 13, 14, 15, 18, and amino acids 1-124 of SEQ ID NO: 129.

6. The method of claim 5, wherein the ISVD is amino acids 1-124 of SEQ ID NO: 129.

7. The method of claim 1, wherein the ADAMTS5 inhibiting polypeptide comprises two or more ISVDS, wherein
a) at least a first ISVD specifically binds a first antigenic determinant, epitope, part, domain, subunit or conformation of ADAMTS5; and wherein,
b) at least a second ISVD specifically binds a second antigenic determinant, epitope, part, domain, subunit or conformation of serum albumin.

8. The method of claim 7, wherein the first ISVD is chosen from the group consisting of SEQ ID NOs: 2, 116, 19, 1, 3, 6, 16, 17, 10, 11, 9, 5, 4, 7, 8, 117, 12, 13, 14, 15, 18, and amino acids 1-124 of SEQ ID NO: 129.

9. The method of claim 8, wherein the first ISVD is amino acids 1-124 of SEQ ID NO: 129.

10. The method of claim 7, wherein the second ISVD is chosen from the group consisting of SEQ ID NOs: 131, 132, 133, 134, 135, 136, 137, 138, 139, 140, 141, 142, 143, 144, or 145.

11. The method of claim 10, wherein the second ISVD is SEQ ID NO: 138.

12. The method of claim 7, wherein the first ISVD is amino acids 1-124 of SEQ ID NO: 129, and the second ISVD is SEQ ID NO: 138.

13. The method of claim 1, wherein the ADAMTS5 inhibiting polypeptide is selected from the group consisting of SEQ ID NO: 129 (clone 577 2F3^{so}-Alb), SEQ ID NO: 130 (clone 579 2F3^{so}-093-Alb), SEQ ID NO: 120 (clone 4 2A12-Alb), SEQ ID NO: 121 (clone 5 2D7-Alb), SEQ ID NO: 122 (clone 6 2F3-Alb), SEQ ID NO: 123 (clone 69 049-Alb), SEQ ID NO: 124 (clone 70 9D3-Alb), SEQ ID NO: 125 (clone 71 3B2-Alb), SEQ ID NO: 126 (clone 129 2F3-093-Alb), SEQ ID NO: 127 (clone 130 049-093-Alb), and SEQ ID NO: 128 (clone 131 9D3-093-Alb).

14. The method of claim 13, wherein the ADAMTS5 inhibiting polypeptide is SEQ ID NO: 129 (clone 577 2F3^{so}-Alb).

15. The method of any of claims 1-14, wherein the osteoarthritis is knee osteoarthritis.

16. The method of any of claims 1-15, wherein the dose of the ADAMTS5 inhibiting polypeptide is 150 mg.

17. The method of any of claims 1-15, wherein the dose of the ADAMTS5 inhibiting polypeptide is 300 mg.

18. The method of any of claims 1-17, wherein the ADAMTS5 inhibiting polypeptide is administered once a month.

19. The method of any of claims 1-18, wherein administration of the ADAMTS5 inhibiting polypeptide decreases the formation of ARGS fragments.

20. The method of any of claims 1-19, wherein administration of the ADAMTS5 inhibiting polypeptide decreases pain in an osteoarthritis affected joint as determined by KOOS score or WOMAC score.

21. The method of any of claims 1-20, wherein administration of the ADAMTS5 inhibiting polypeptide maintains the structure of an osteoarthritis affected joint as determined by automated segmentation in MRI.


SEQUENCE LISTING (175 sequences)

SEQ ID NO: 1 - protein, 124 aa, Artificial Sequence
  Description of Artificial Sequence Synthetic polypeptide
EVQLVESGGGLVQAGGSLRLSCAASGRTFSSYAMGWFRQAPGKERDFVAGISRSAGRTYYVDSVKGRFTISRDSAKNTVYLQMNRLKPEDTAVYYCAADLDPNRIFSRDEAAYWGQGTLVTVSS

SEQ ID NO: 2 - protein, 124 aa, Artificial Sequence
  Description of Artificial Sequence Synthetic polypeptide
EVQLVESGGGLVQAGGSLRLSCAASGRTVSSYAMGWFRLAPGKEREFVAGISRSAERTYYVDSLKGRFTISRDSAKNTVYLHMNRLKPEDTAVYYCAADLDPNRIFSREEYAYWGQGTLVTVSS

SEQ ID NO: 3 - protein, 124 aa, Artificial Sequence
  Description of Artificial Sequence Synthetic polypeptide
EVQLVESGGGLVQSGGSLRLSCAASGSAVSVNAMAWYRQAPGKQRDFVAGISRSAGRTYYTDSVKDRFTIARDSAKNTVYLQMNRLKPEDTAVYYCAADLDPNRIFSRDEAAYWGQGTLVTVSS

SEQ ID NO: 4 - protein, 125 aa, Artificial Sequence
  Description of Artificial Sequence Synthetic polypeptide
EVQLVESGGGLVQAGDSLRLSCAASGRTFSTYFVGWFRQAPGKERDFVAAISRNGARTYYYDSVAGLFTISRDNAKNTVYLQMSSLKPEDTAVYYCAAARISPSDPSNEDGYDYWGQGTLVTVSS

SEQ ID NO: 5 - protein, 126 aa, Artificial Sequence
  Description of Artificial Sequence Synthetic polypeptide
EVQLVESGGGLVQPGGSLRLSCAASRRTFSSYVMAWFRQAPGKEREFVAAISRSGDSTYYYDSLEGRFTISRDNAKNTVHLQMNSLKPEDTAVYICAASRAPSFRTIDAINYYDYWGQGTLVTVSS

SEQ ID NO: 6 - protein, 126 aa, Artificial Sequence
  Description of Artificial Sequence Synthetic polypeptide
EVQLVESGGGLVQAGGSLRLSCSFSGPGRTFARYAMGWFRQAPGKNRDFITGISGSGDSTYYVYPMKDRFTISRDNAKNMVYLQMNALKPEDTAVYYCAADREINRIANDKELDFWGQGTLVTVSS

SEQ ID NO: 7 - protein, 123 aa, Artificial Sequence
  Description of Artificial Sequence Synthetic polypeptide
EVQLVESGGGLVQAGGSLRLSCAASGRTTFSSYAMGWFRQAPGKERAFVATIWSGGLTVYADSAKGRFTISRDNAKNTVYLQMNSLRPEDTAVYYCAAEAVGTYYTPDGWTYWGQGTLVTVSS

SEQ ID NO: 8 - protein, 124 aa, Artificial Sequence
  Description of Artificial Sequence Synthetic polypeptide
EVQLVESGGGLVQAGGSLRLSCAASGLTFRRNAMGWFRQAPGKERELLAGINWSGGTTYYVDSVKGRFTISRDNAKNTVDLQMISPKPEDTAVYYCAADGDIGTLVNDENPRYWGQGTLVTVSS

SEQ ID NO: 9 - protein, 124 aa, Artificial Sequence
  Description of Artificial Sequence Synthetic polypeptide
EVQLVESGGGLVQAGGSLRLSCATSGFTFSPYYMGWFRQAPGKERDFVAAITRSRGTTYYLDSTEGRFTISRDNAKNTMYLQMNSLNPEDTAVYYCAAGRSPGDPSRTYLYEYWGQGTLVTVSS

SEQ ID NO: 10 - protein, 124 aa, Artificial Sequence
  Description of Artificial Sequence Synthetic polypeptide
EVQLVESGGGLVQAGGSLRLSCAPSGFTFSPYYMGWFRQAPGKERDFVAAISRSRGTTYYLDSTEGRFTISRDNANDTVYLQMNSLNPEDTAVYYCAAGRSPGDPSRTYLYDYWGQGTLVTVSS

SEQ ID NO: 11 - protein, 124 aa, Artificial Sequence
  Description of Artificial Sequence Synthetic polypeptide
EVQLVESGGGLVQAGASLRLSCATSGFTFSPYYMGWFRQAPGKERDFVAAISWSRGILYYTDSTEGRFTISRDNAKNTMYLQMDNLNPEDTAVYYCAASRSPGDPSRTYLYDYWGQGTLVTVSS

SEQ ID NO: 12 - protein, 127 aa, Artificial Sequence
  Description of Artificial Sequence Synthetic polypeptide
EVQLVESGGGLVQAGGSLRLSCAASGRALSSSIMGWFRQAPGKEREFVAAITWSGGRAYYADVSDFEKGRFTISRDNGKNTVNLQMKGLKPEDTAVYYCAAALAIPVTMSPHEYPYWGQGTLVTVSS

SEQ ID NO: 13 - protein, 121 aa, Artificial Sequence
  Description of Artificial Sequence Synthetic polypeptide
EVQLVESGGDLVQPGGSLRLSCAASGSDVVVNDMGWYRQAPGKQRELVADITTGGRTNYADSVKGRFTISRDNVKNTVYLQMNSLKPEDTAVYYCNAQVGDSDDDVWYAYWGQGTLVTVSS

SEQ ID NO: 14 - protein, 119 aa, Artificial Sequence
  Description of Artificial Sequence Synthetic polypeptide
EVQLVESGGGLVQPGGSLRLSCAASGSIFSINVMGWYRQAPGKQRELVAAIISGGRTNYADSVKGRFTISRDNSKNTVYLQMNSLRPEDTAVYYCNAEVDAGIYAYGYWGQGTLVTVSS

SEQ ID NO: 15 - protein, 124 aa, Artificial Sequence
  Description of Artificial Sequence Synthetic polypeptide
EVQLVESGGGLVQAGGSLRLSCAASGLTFSSYTMGWFRQAPGQEREFVSAISWNTFTTYYVDSVKDRFTVSRDNAKNTLYLRMNSLKPEDTAVYYCAAAGGSPRQHEPYEYRVWGQGTLVTVSS

SEQ ID NO: 16 - protein, 125 aa, Artificial Sequence
  Description of Artificial Sequence Synthetic polypeptide
EVQLVESGGGFVQAGGSLRLSCVASRRTISSGTMGWFRQAPGKEREFVAAIRWSSGMPYYLDSVMDRFTISRDNAKNTVSLQMNSLQPEDTAVYYCAADRSAFRDPSFDVNYEYWGRGTLVTVSS

SEQ ID NO: 17 - protein, 125 aa, Artificial Sequence
  Description of Artificial Sequence Synthetic polypeptide
EVQLVESGGGFVQAGGSLRLSCAASRRTISSGTMGWFRQAPGKEREFVAAIRWSSGITFYPDSVEGRFTISGDNAKNTVSLQMNSLKPEDTAVYYCAADRSALRDPSFEVNYEYWGRGTLVTVSS

SEQ ID NO: 18 - protein, 120 aa, Artificial Sequence
  Description of Artificial Sequence Synthetic polypeptide
EVQLVESGGGLVQAGGSLRLSCVASGSIFSIDAMGWYRQAPGKERELVASVTTGASPNYGDSVTGRFTASRDRAKNALYLQMNSLKPEDTAVYYCNLIMTIPGGSQIMYWGQGTLVTVSS

SEQ ID NO: 19 - protein, 120 aa, Artificial Sequence
  Description of Artificial Sequence Synthetic polypeptide
EVQLVESGGGSVQAGGSLRLSCVASGRYPMAWFRQAPGKEREFVAGVSWGGDRTYYADSVQGRFTVSRDYAKNTLYLQMNSLKPEDAAVYYCAGDPWGRLFRVKDNYSDWGQGTLVTVSS

SEQ ID NO: 20 - protein, 10 aa, Artificial Sequence
  Description of Artificial Sequence Synthetic peptide
GRTFSSYAMG

SEQ ID NO: 21 - protein, 10 aa, Artificial Sequence
  Description of Artificial Sequence Synthetic peptide
GRTVSSYAMG

SEQ ID NO: 22 - protein, 10 aa, Artificial Sequence
  Description of Artificial Sequence Synthetic peptide
GSAVSVNAMA

SEQ ID NO: 23 - protein, 10 aa, Artificial Sequence
  Description of Artificial Sequence Synthetic peptide
GRTFSTYFVG

SEQ ID NO: 24 - protein, 10 aa, Artificial Sequence
  Description of Artificial Sequence Synthetic peptide
RRTFSSYVMA

SEQ ID NO: 25 - protein, 12 aa, Artificial Sequence
  Description of Artificial Sequence Synthetic peptide
GPGRTFARYAMG

SEQ ID NO: 26 - protein, 11 aa, Artificial Sequence
  Description of Artificial Sequence Synthetic peptide
GRTTFSSYAMG

SEQ ID NO: 27 - protein, 10 aa, Artificial Sequence
  Description of Artificial Sequence Synthetic peptide
GLTFRRNAMG

SEQ ID NO: 28 - protein, 10 aa, Artificial Sequence
  Description of Artificial Sequence Synthetic peptide
GFTFSPYYMG

SEQ ID NO: 29 - protein, 10 aa, Artificial Sequence
  Description of Artificial Sequence Synthetic peptide
GRALSSSIMG

SEQ ID NO: 30 - protein, 10 aa, Artificial Sequence
  Description of Artificial Sequence Synthetic peptide
GSDVVVNDMG

SEQ ID NO: 31 - protein, 10 aa, Artificial Sequence
  Description of Artificial Sequence Synthetic peptide
GSIFSINVMG

SEQ ID NO: 32 - protein, 10 aa, Artificial Sequence
  Description of Artificial Sequence Synthetic peptide
GLTFSSYTMG

SEQ ID NO: 33 - protein, 10 aa, Artificial Sequence
  Description of Artificial Sequence Synthetic peptide
RRTISSGTMG

SEQ ID NO: 34 - protein, 10 aa, Artificial Sequence
  Description of Artificial Sequence Synthetic peptide
GSIFSIDAMG

SEQ ID NO: 35 - protein, 6 aa, Artificial Sequence
  Description of Artificial Sequence Synthetic peptide
GRYPMA

SEQ ID NO: 36 - protein, 10 aa, Artificial Sequence
  Description of Artificial Sequence Synthetic peptide
GISRSAGRTY

SEQ ID NO: 37 - protein, 10 aa, Artificial Sequence
  Description of Artificial Sequence Synthetic peptide
GISRSAERTY

SEQ ID NO: 38 - protein, 11 aa, Artificial Sequence
  Description of Artificial Sequence Synthetic peptide
AISRNGARTYY

SEQ ID NO: 39 - protein, 10 aa, Artificial Sequence
  Description of Artificial Sequence Synthetic peptide
AISRSGDSTY

SEQ ID NO: 40 - protein, 10 aa, Artificial Sequence
  Description of Artificial Sequence Synthetic peptide
GISGSGDSTY

SEQ ID NO: 41 - protein, 9 aa, Artificial Sequence
  Description of Artificial Sequence Synthetic peptide
TIWSGGLTV

SEQ ID NO: 42 - protein, 10 aa, Artificial Sequence
  Description of Artificial Sequence Synthetic peptide
GINWSGGTTY

SEQ ID NO: 43 - protein, 10 aa, Artificial Sequence
  Description of Artificial Sequence Synthetic peptide
AITRSRGTTY

SEQ ID NO: 44 - protein, 10 aa, Artificial Sequence
  Description of Artificial Sequence Synthetic peptide
AISRSRGTTY

SEQ ID NO: 45 - protein, 10 aa, Artificial Sequence
  Description of Artificial Sequence Synthetic peptide
AISWSRGILY

SEQ ID NO: 46 - protein, 13 aa, Artificial Sequence
  Description of Artificial Sequence Synthetic peptide
AITWSGGRAYYAD

SEQ ID NO: 47 - protein, 9 aa, Artificial Sequence
  Description of Artificial Sequence Synthetic peptide
DITTGGRTN

SEQ ID NO: 48 - protein, 9 aa, Artificial Sequence
  Description of Artificial Sequence Synthetic peptide
AIISGGRTN

SEQ ID NO: 49 - protein, 10 aa, Artificial Sequence
  Description of Artificial Sequence Synthetic peptide
AISWNTFTTY

SEQ ID NO: 50 - protein, 10 aa, Artificial Sequence
  Description of Artificial Sequence Synthetic peptide
AIRWSSGMPY

SEQ ID NO: 51 - protein, 10 aa, Artificial Sequence
  Description of Artificial Sequence Synthetic peptide
AIRWSSGITF

SEQ ID NO: 52 - protein, 9 aa, Artificial Sequence
  Description of Artificial Sequence Synthetic peptide
SVTTGASPN

SEQ ID NO: 53 - protein, 10 aa, Artificial Sequence
  Description of Artificial Sequence Synthetic peptide
GVSWGGDRTY

SEQ ID NO: 54 - protein, 15 aa, Artificial Sequence
  Description of Artificial Sequence Synthetic peptide
DLDPNRIFSRDEAAY

SEQ ID NO: 55 - protein, 15 aa, Artificial Sequence
  Description of Artificial Sequence Synthetic peptide
DLDPNRIFSREEYAY

SEQ ID NO: 56 - protein, 16 aa, Artificial Sequence
  Description of Artificial Sequence Synthetic peptide
ARISPSDPSNEDGYDY

SEQ ID NO: 57 - protein, 17 aa, Artificial Sequence
  Description of Artificial Sequence Synthetic peptide
SRAPSFRTIDAINYYDY

SEQ ID NO: 58 - protein, 15 aa, Artificial Sequence
  Description of Artificial Sequence Synthetic peptide
DREINRIANDKELDF

SEQ ID NO: 59 - protein, 14 aa, Artificial Sequence
  Description of Artificial Sequence Synthetic peptide
EAVGTYYTPDGWTY

SEQ ID NO: 60 - protein, 15 aa, Artificial Sequence
  Description of Artificial Sequence Synthetic peptide
DGDIGTLVNDENPRY

SEQ ID NO: 61 - protein, 15 aa, Artificial Sequence
  Description of Artificial Sequence Synthetic peptide
GRSPGDPSRTYLYEY

SEQ ID NO: 62 - protein, 15 aa, Artificial Sequence
  Description of Artificial Sequence Synthetic peptide
GRSPGDPSRTYLYDY

SEQ ID NO: 63 - protein, 15 aa, Artificial Sequence
  Description of Artificial Sequence Synthetic peptide
SRSPGDPSRTYLYDY

SEQ ID NO: 64 - protein, 15 aa, Artificial Sequence
  Description of Artificial Sequence Synthetic peptide
ALAIPVTMSPHEYPY

SEQ ID NO: 65 - protein, 13 aa, Artificial Sequence
  Description of Artificial Sequence Synthetic peptide
QVGDSDDDVWYAY

SEQ ID NO: 66 - protein, 11 aa, Artificial Sequence
  Description of Artificial Sequence Synthetic peptide
EVDAGIYAYGY

SEQ ID NO: 67 - protein, 15 aa, Artificial Sequence
  Description of Artificial Sequence Synthetic peptide
AGGSPRQHEPYEYRV

SEQ ID NO: 68 - protein, 16 aa, Artificial Sequence
  Description of Artificial Sequence Synthetic peptide
DRSAFRDPSFDVNYEY

SEQ ID NO: 69 - protein, 16 aa, Artificial Sequence
  Description of Artificial Sequence Synthetic peptide
DRSALRDPSFEVNYEY

SEQ ID NO: 70 - protein, 12 aa, Artificial Sequence
  Description of Artificial Sequence Synthetic peptide
IMTIPGGSQIMY

SEQ ID NO: 71 - protein, 15 aa, Artificial Sequence
  Description of Artificial Sequence Synthetic peptide
DPWGRLFRVKDNYSD

SEQ ID NO: 72 - protein, 25 aa, Artificial Sequence
  Description of Artificial Sequence Synthetic peptide
EVQLVESGGGLVQAGGSLRLSCAAS

SEQ ID NO: 73 - protein, 25 aa, Artificial Sequence
  Description of Artificial Sequence Synthetic peptide
EVQLVESGGGLVQSGGSLRLSCAAS

SEQ ID NO: 74 - protein, 25 aa, Artificial Sequence
  Description of Artificial Sequence Synthetic peptide
EVQLVESGGGLVQAGDSLRLSCAAS

SEQ ID NO: 75 - protein, 25 aa, Artificial Sequence [sequence omitted]

SEQ ID NO: 76 - protein, 25 aa, Artificial Sequence
  Description of Artificial Sequence Synthetic peptide
EVQLVESGGGLVQAGGSLRLSCSFS

SEQ ID NO: 77 - protein, 25 aa, Artificial Sequence
  Description of Artificial Sequence Synthetic peptide
EVQLVESGGGLVQAGGSLRLSCATS

SEQ ID NO: 78 - protein, 25 aa, Artificial Sequence
  Description of Artificial Sequence Synthetic peptide
EVQLVESGGGLVQAGGSLRLSCAPS

SEQ ID NO: 79 - protein, 25 aa, Artificial Sequence
  Description of Artificial Sequence Synthetic peptide
EVQLVESGGGLVQAGASLRLSCATS

SEQ ID NO: 80 - protein, 25 aa, Artificial Sequence
  Description of Artificial Sequence Synthetic peptide
EVQLVESGGDLVQPGGSLRLSCAAS

SEQ ID NO: 81 - protein, 25 aa, Artificial Sequence
  Description of Artificial Sequence Synthetic peptide
EVQLVESGGGFVQAGGSLRLSCVAS

SEQ ID NO: 82 - protein, 25 aa, Artificial Sequence
  Description of Artificial Sequence Synthetic peptide
EVQLVESGGGFVQAGGSLRLSCAAS

SEQ ID NO: 83 - protein, 25 aa, Artificial Sequence
  Description of Artificial Sequence Synthetic peptide
EVQLVESGGGLVQAGGSLRLSCVAS

SEQ ID NO: 84 - protein, 25 aa, Artificial Sequence
  Description of Artificial Sequence Synthetic peptide
EVQLVESGGGSVQAGGSLRLSCVAS

SEQ ID NO: 85 - protein, 14 aa, Artificial Sequence
  Description of Artificial Sequence Synthetic peptide
WFRQAPGKERDFVA

SEQ ID NO: 86 - protein, 14 aa, Artificial Sequence
  Description of Artificial Sequence Synthetic peptide
WFRLAPGKEREFVA

SEQ ID NO: 87 - protein, 14 aa, Artificial Sequence
  Description of Artificial Sequence Synthetic peptide
WYRQAPGKQRDFVA

SEQ ID NO: 88 - protein, 14 aa, Artificial Sequence
  Description of Artificial Sequence Synthetic peptide
WFRQAPGKEREFVA

SEQ ID NO: 89 - protein, 14 aa, Artificial Sequence
  Description of Artificial Sequence Synthetic peptide
WFRQAPGKNRDFIT

SEQ ID NO: 90 - protein, 14 aa, Artificial Sequence
  Description of Artificial Sequence Synthetic peptide
WFRQAPGKERAFVA

SEQ ID NO: 91 - protein, 14 aa, Artificial Sequence
  Description of Artificial Sequence Synthetic peptide
WFRQAPGKERELLA

SEQ ID NO: 92 - protein, 14 aa, Artificial Sequence
  Description of Artificial Sequence Synthetic peptide
WYRQAPGKQRELVA

SEQ ID NO: 93 - protein, 14 aa, Artificial Sequence
  Description of Artificial Sequence Synthetic peptide
WFRQAPGQEREFVS

SEQ ID NO: 94 - protein, 14 aa, Artificial Sequence
  Description of Artificial Sequence Synthetic peptide
WYRQAPGKERELVA

SEQ ID NO: 95 - protein, 39 aa, Artificial Sequence
  Description of Artificial Sequence Synthetic polypeptide
YVDSVKGRFTISRDSAKNTVYLQMNRLKPEDTAVYYCAA

SEQ ID NO: 96 - protein, 39 aa, Artificial Sequence
  Description of Artificial Sequence Synthetic polypeptide
YVDSLKGRFTISRDSAKNTVYLHMNRLKPEDTAVYYCAA

SEQ ID NO: 97 - protein, 39 aa, Artificial Sequence
  Description of Artificial Sequence Synthetic polypeptide
YTDSVKDRFTIARDSAKNTVYLQMNRLKPEDTAVYYCAA

SEQ ID NO: 98 - protein, 38 aa, Artificial Sequence
  Description of Artificial Sequence Synthetic polypeptide
YDSVAGLFTISRDNAKNTVYLQMSSLKPEDTAVYYCAA

SEQ ID NO: 99 - protein, 39 aa, Artificial Sequence
  Description of Artificial Sequence Synthetic polypeptide
YYDSLEGRFTISRDNAKNTVHLQMNSLKPEDTAVYICAA

SEQ ID NO: 100 - protein, 39 aa, Artificial Sequence
  Description of Artificial Sequence Synthetic polypeptide
YVYPMKDRFTISRDNAKNMVYLQMNALKPEDTAVYYCAA

SEQ ID NO: 101 - protein, 39 aa, Artificial Sequence
  Description of Artificial Sequence Synthetic polypeptide
YADSAKGRFTISRDNAKNTVYLQMNSLRPEDTAVYYCAA

SEQ ID NO: 102 - protein, 39 aa, Artificial Sequence
  Description of Artificial Sequence Synthetic polypeptide
YVDSVKGRFTISRDNAKNTVDLQMISPKPEDTAVYYCAA

SEQ ID NO: 103 - protein, 39 aa, Artificial Sequence
  Description of Artificial Sequence Synthetic polypeptide
YLDSTEGRFTISRDNAKNTMYLQMNSLNPEDTAVYYCAA

SEQ ID NO: 104 - protein, 39 aa, Artificial Sequence
  Description of Artificial Sequence Synthetic polypeptide
YLDSTEGRFTISRDNANDTVYLQMNSLNPEDTAVYYCAA

SEQ ID NO: 105 - protein, 39 aa, Artificial Sequence
  Description of Artificial Sequence Synthetic polypeptide
YTDSTEGRFTISRDNAKNTMYLQMDNLNPEDTAVYYCAA

SEQ ID NO: 106 - protein, 39 aa, Artificial Sequence
  Description of Artificial Sequence Synthetic polypeptide
VSDFEKGRFTISRDNGKNTVNLQMKGLKPEDTAVYYCAA

SEQ ID NO: 107 - protein, 39 aa, Artificial Sequence
  Description of Artificial Sequence Synthetic polypeptide
YADSVKGRFTISRDNVKNTVYLQMNSLKPEDTAVYYCNA

SEQ ID NO: 108 - protein, 39 aa, Artificial Sequence
  Description of Artificial Sequence Synthetic polypeptide
YADSVKGRFTISRDNSKNTVYLQMNSLRPEDTAVYYCNA

SEQ ID NO: 109 - protein, 39 aa, Artificial Sequence
  Description of Artificial Sequence Synthetic polypeptide
YVDSVKDRFTVSRDNAKNTLYLRMNSLKPEDTAVYYCAA

SEQ ID NO: 110 - protein, 39 aa, Artificial Sequence
  Description of Artificial Sequence Synthetic polypeptide
YLDSVMDRFTISRDNAKNTVSLQMNSLQPEDTAVYYCAA

SEQ ID NO: 111 - protein, 39 aa, Artificial Sequence
  Description of Artificial Sequence Synthetic polypeptide
YPDSVEGRFTISGDNAKNTVSLQMNSLKPEDTAVYYCAA

SEQ ID NO: 112 - protein, 39 aa, Artificial Sequence
  Description of Artificial Sequence Synthetic polypeptide
YGDSVTGRFTASRDRAKNALYLQMNSLKPEDTAVYYCNL

SEQ ID NO: 113 - protein, 39 aa, Artificial Sequence
  Description of Artificial Sequence Synthetic polypeptide
YADSVQGRFTVSRDYAKNTLYLQMNSLKPEDAAVYYCAG

SEQ ID NO: 114 - protein, 11 aa, Artificial Sequence
  Description of Artificial Sequence Synthetic peptide
WGQGTLVTVSS

SEQ ID NO: 115 - protein, 11 aa, Artificial Sequence
  Description of Artificial Sequence Synthetic peptide
WGRGTLVTVSS

SEQ ID NO: 116 - protein, 120 aa, Artificial Sequence
  Description of Artificial Sequence Synthetic polypeptide
EVQLVESGGGSVQAGGSLRLSCVASGRYPMAWFRQAPGKEREFVAGVSWGGDRTYYADSVQGRFTVSRDYAKNTLYLQMNSLKPEDAAVYYCAGDPWGRLFRVKDQYSDWGQGTLVTVSS

SEQ ID NO: 117 - protein, 124 aa, Artificial Sequence
  Description of Artificial Sequence Synthetic polypeptide
EVQLVESGGGLVQAGGSLRLSCAASGLTFRRNAMGWFRQAPGKERELLAGISWSGGTTYYVDSVKGRFTISRDNAKNTVDLQMISPKPEDTAVYYCAADGDIGTLVNDENPRYWGQGTLVTVSS

SEQ ID NO: 118 - protein, 15 aa, Artificial Sequence
  Description of Artificial Sequence Synthetic peptide
DPWGRLFRVKDQYSD

SEQ ID NO: 119 - protein, 10 aa, Artificial Sequence
  Description of Artificial Sequence Synthetic peptide
GISWSGGTTY

SEQ ID NO: 120 - protein, 274 aa, Artificial Sequence
  Description of Artificial Sequence Synthetic polypeptide
EVQLVESGGGLVQAGGSLRLSCAASGRTFSSYAMGWFRQAPGKERDFVAGISRSAGRTYYVDSVKGRFTISRDSAKNTVYLQMNRLKPEDTAVYYCAADLDPNRIFSRDEAAYWGQGTLVTVSSGGGGSGGGGSGGGGSGGGGSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 121 - protein, 276 aa, Artificial Sequence
  Description of Artificial Sequence Synthetic polypeptide
EVQLVESGGGLVQAGGSLRLSCSFSGPGRTFARYAMGWFRQAPGKNRDFITGISGSGDSTYYVYPMKDRFTISRDNAKNMVYLQMNALKPEDTAVYYCAADREINRIANDKELDFWGQGTLVTVSSGGGGSGGGGSGGGGSGGGGSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 122 - protein, 274 aa, Artificial Sequence
  Description of Artificial Sequence Synthetic polypeptide
EVQLVESGGGLVQAGGSLRLSCAASGRTVSSYAMGWFRLAPGKEREFVAGISRSAERTYYVDSLKGRFTISRDSAKNTVYLHMNRLKPEDTAVYYCAADLDPNRIFSREEYAYWGQGTLVTVSSGGGGSGGGGSGGGGSGGGGSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 123 - protein, 274 aa, Artificial Sequence
  Description of Artificial Sequence Synthetic polypeptide
EVQLVESGGGLVQAGGSLRLSCAASGLTFRRNAMGWFRQAPGKERELLAGISWSGGTTYYVDSVKGRFTISRDNAKNTVDLQMISPKPEDTAVYYCAADGDIGTLVNDENPRYWGQGTLVTVSSGGGGSGGGGSGGGGSGGGGSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 124 - protein, 274 aa, Artificial Sequence
  Description of Artificial Sequence Synthetic polypeptide
EVQLVESGGGLVQSGGSLRLSCAASGSAVSVNAMAWYRQAPGKQRDFVAGISRSAGRTYYTDSVKDRFTIARDSAKNTVYLQMNRLKPEDTAVYYCAADLDPNRIFSRDEAAYWGQGTLVTVSSGGGGSGGGGSGGGGSGGGGSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 125 - protein, 275 aa, Artificial Sequence
  Description of Artificial Sequence Synthetic polypeptide
EVQLVESGGGFVQAGGSLRLSCVASRRTISSGTMGWFRQAPGKEREFVAAIRWSSGMPYYLDSVMDRFTISRDNAKNTVSLQMNSLQPEDTAVYYCAADRSAFRDPSFDVNYEYWGRGTLVTVSSGGGGSGGGGSGGGGSGGGGSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 126 - protein, 429 aa, Artificial Sequence
  Description of Artificial Sequence Synthetic polypeptide
EVQLVESGGGLVQAGGSLRLSCAASGRTVSSYAMGWFRLAPGKEREFVAGISRSAERTYYVDSLKGRFTISRDSAKNTVYLHMNRLKPEDTAVYYCAADLDPNRIFSREEYAYWGQGTLVTVSSGGGGSGGGGSGGGGSGGGGSGGGGSGGGGSGGGGSEVQLVESGGGSVQAGGSLRLSCVASGRYPMAWFRQAPGKEREFVAGVSWGGDRTYYADSVQGRFTVSRDYAKNTLYLQMNSLKPEDAAVYYCAGDPWGRLFRVKDQYSDWGQGTLVTVSSGGGGSGGGGSGGGGSGGGGSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 127 - protein, 429 aa, Artificial Sequence
  Description of Artificial Sequence Synthetic polypeptide
EVQLVESGGGLVQAGGSLRLSCAASGLTFRRNAMGWFRQAPGKERELLAGISWSGGTTYYVDSVKGRFTISRDNAKNTVDLQMISPKPEDTAVYYCAADGDIGTLVNDENPRYWGQGTLVTVSSGGGGSGGGGSGGGGSGGGGSGGGGSGGGGSGGGGSEVQLVESGGGSVQAGGSLRLSCVASGRYPMAWFRQAPGKEREFVAGVSWGGDRTYYADSVQGRFTVSRDYAKNTLYLQMNSLKPEDAAVYYCAGDPWGRLFRVKDQYSDWGQGTLVTVSSGGGGSGGGGSGGGGSGGGGSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 128 - protein, 429 aa, Artificial Sequence
  Description of Artificial Sequence Synthetic polypeptide
EVQLVESGGGLVQSGGSLRLSCAASGSAVSVNAMAWYRQAPGKQRDFVAGISRSAGRTYYTDSVKDRFTIARDSAKNTVYLQMNRLKPEDTAVYYCAADLDPNRIFSRDEAAYWGQGTLVTVSSGGGGSGGGGSGGGGSGGGGSGGGGSGGGGSGGGGSEVQLVESGGGSVQAGGSLRLSCVASGRYPMAWFRQAPGKEREFVAGVSWGGDRTYYADSVQGRFTVSRDYAKNTLYLQMNSLKPEDAAVYYCAGDPWGRLFRVKDQYSDWGQGTLVTVSSGGGGSGGGGSGGGGSGGGGSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 129 - protein, 275 aa, Artificial Sequence
  Description of Artificial Sequence Synthetic polypeptide
DVQLVESGGGVVQPGGSLRLSCAASGRTVSSYAMGWFRQAPGKEREFVAGISRSAERTYYVDSLKGRFTISRDNSKNTVYLQMNSLRPEDTALYYCAADLDPNRIFSREEYAYWGQGTLVTVSSGGGGSGGGGSGGGGSGGGGSGGGGSGGGGSGGGGSEVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 130 - protein, 430 aa, Artificial Sequence
  Description of Artificial Sequence Synthetic polypeptide
DVQLVESGGGVVQPGGSLRLSCAASGRTVSSYAMGWFRQAPGKEREFVAGISRSAERTYYVDSLKGRFTISRDNSKNTVYLQMNSLRPEDTALYYCAADLDPNRIFSREEYAYWGQGTLVTVSSGGGGSGGGGSGGGGSGGGGSGGGGSGGGGSGGGGSEVQLVESGGGVVQPGGSLRLSCAASGRYPMAWFRQAPGKEREFVAGVSWGGDRTYYADSVKGRFTISRDYSKNTLYLQMNSLRPEDTALYYCAGDPFGRLFRVKDQYSDWGQGTLVTVSSGGGGSGGGGSGGGGSGGGGSGGGGSGGGGSGGGGSEVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 131 - protein, 115 aa, Artificial Sequence [sequence omitted]

SEQ ID NO: 132 - protein, 115 aa, Artificial Sequence [sequence omitted]

SEQ ID NO: 133 - protein, 116 aa, Artificial Sequence
  Description of Artificial Sequence Synthetic polypeptide
EVQLVESGGGVVQPGNSLRLSCAASGFTFSSFGMSWVRQAPGKGLEWVSSISGSGSDTLYADSVKGRFTISRDNAKTTLYLQMNSLRPEDTATYYCTIGGSLSRSSQGTLVTVSSA

SEQ ID NO: 134 - protein, 116 aa, Artificial Sequence
  Description of Artificial Sequence Synthetic polypeptide
EVQLVESGGGVVQPGGSLRLSCAASGFTFRSFGMSWVRQAPGKGPEWVSSISGSGSDTLYADSVKGRFTISRDNSKNTLYLQMNSLRPEDTATYYCTIGGSLSRSSQGTLVTVSSA

SEQ ID NO: 135 - protein, 115 aa, Artificial Sequence [sequence omitted]

SEQ ID NO: 136 - protein, 116 aa, Artificial Sequence
  Description of Artificial Sequence Synthetic polypeptide
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVKVSSA

SEQ ID NO: 137 - protein, 115 aa, Artificial Sequence
  Description of Artificial Sequence Synthetic polypeptide
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSS

SEQ ID NO: 138 - protein, 116 aa, Artificial Sequence
  Description of Artificial Sequence Synthetic polypeptide
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 139 - protein, 117 aa, Artificial Sequence
  Description of Artificial Sequence Synthetic polypeptide
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAA

SEQ ID NO: 140 - protein, 118 aa, Artificial Sequence
  Description of Artificial Sequence Synthetic polypeptide
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAAA

SEQ ID NO: 141 - protein, 116 aa, Artificial Sequence
  Description of Artificial Sequence Synthetic polypeptide
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSG

SEQ ID NO: 142 - protein, 117 aa, Artificial Sequence
  Description of Artificial Sequence Synthetic polypeptide
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGG

SEQ ID NO: 143 - protein, 118 aa, Artificial Sequence
  Description of Artificial Sequence Synthetic polypeptide
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGGG

SEQ ID NO: 144 - protein, 115 aa, Artificial Sequence
  Description of Artificial Sequence Synthetic polypeptide
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 145 - protein, 116 aa, Artificial Sequence
  Description of Artificial Sequence Synthetic polypeptide
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 146 - protein, 5 aa, Artificial Sequence
  Description of Artificial Sequence Synthetic peptide
SFGMS

SEQ ID NO: 147 - protein, 17 aa, Artificial Sequence
  Description of Artificial Sequence Synthetic peptide
SISGSGSDTLYADSVKG

SEQ ID NO: 148 - protein, 6 aa, Artificial Sequence
  Description of Artificial Sequence Synthetic peptide
GGSLSR

SEQ ID NO: 149 - protein, 930 aa, Homo sapiens
MLLGWASLLLCAFRLPLAAVGPAATPAQDKAGQPPTAAAAAQPRRRQGEEVQERAEPPGHPHPLAQRRRSKGLVQNIDQLYSGGGKVGYLVYAGGRRFLLDLERDGSVGIAGFVPAGGGTSAPWRHRSHCFYRGTVDGSPRSLAVFDLCGGLDGFFAVKHARYTLKPLLRGPWAEEEKGRVYGDGSARILHVYTREGFSFEALPPRASCETPASTPEAHEHAPAHSNPSGRAALASQLLDQSALSPAGGSGPQTWWRRRRRSISRARQVELLLVADASMARLYGRGLQHYLLTLASIANRLYSHASIENHIRLAVVKVVVLGDKDKSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEETFGSTEDKRLMSSILTSIDASKPWSKCTSATITEFLDDGHGNCLLDLPRKQILGPEELPGQTYDATQQCNLTFGPEYSVCPGMDVCARLWCAVVRQGQMVCLTKKLPAVEGTPCGKGRICLQGKCVDKTKKKYYSTSSHGNWGSWGSWGQCSRSCGGGVQFAYRHCNNPAPRNNGRYCTGKRAIYRSCSLMPCPPNGKSFRHEQCEAKNGYQSDAKGVKTFVEWVPKYAGVLPADVCKLTCRAKGTGYYVVFSPKVTDGTECRLYSNSVCVRGKCVRTGCDGIIGSKLQYDKCGVCGGDNSSCTKIVGTFNKKSKGYTDVVRIPEGATHIKVRQFKAKDQTRFTAYLALKKKNGEYLINGKYMISTSETIIDINGTVMNYSGWSHRDDFLHGMGYSATKEILIVQILATDPTKPLDVRYSFFVPKKSTPKVNSVTSHGSNKVGSHTSQPQWVTGPWLACSRTCDTGWHTRTVQCQDGNRKLAKGCPLSQRPSAFKQCLLKKC

SEQ ID NO: 150 - protein, 636 aa, Bos taurus
MLLGWAALMLCALRLPPVAAGPTAAPAQDKAGQPRAAAVAAAAQPRGRRGEEAQEPAEPPGHPHPLAPQRGSRGLVQNIDQLYSGGGKVGYLVYAGGRRFLLDLERDDSVGAAGLVPAGGGPNATRRHRGHCFYRGTVDGSPRSLAVFDLCGGLDGFFAVKRARYTLQPLLRGPWAEAEGDARVYGDESARILHVYTREGFSFEALPPRTSCETHASPPGARERPPAPSRPDGRWALAPQQLPGQSAPSSDGSQGPRTWWRRRRRSISRARQVELLLVADASMARMYGRGLQHYLLTLASIANKLYSHASIENHIRLVVVKVVVLGDKDKSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEENFGSTEDKRLMSSILTSIDASKPWSKCTSATITEFLDDGHGNCLLDLPRKQIPGPEELPGQTYDASQQCNLTFGPEYSVCPGMDVCARLWCAVVRQGQMVCLTKKLPAVEGTPCGKGRICLQGKCVDKTKKKYYSTSSHGNWGSWGSWGQCSRSCGGGVQFAYRHCNNPAPRNNGRYCTGKRAIYRSCSVTPCPHHHHHHHHHH

SEQ ID NO: 151 - protein, 629 aa, Rattus norvegicus
MRLEWASLLLLLLLLCASCLALAADNPAAAPAQDKTRQPRAAAAAAQPDQRQWEETQERGHPQPLARQRRSSGLVQNIDQLYSGGGKVGYLVYAGGRRFLLDLERDDTVGAAGGIVTAGGLSASSGHRGHCFYRGTVDGSPRSLAVFDLCGGLDGFFAVKHARYTLKPLLRGSWAESERVYGDGSSRILHVYTREGFSFEALPPRTSCETPASPSGAQESPSVHSSSRRRTELAPQLLDHSAFSPAGNAGPQTWWRRRRRSISRARQVELLLVADSSMAKMYGRGLQHYLLTLASIANRLYSHASIENHIRLAVVKVVVLTDKSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEENFGSTEDKRLMSSILTSIDASKPWSKCTSATITEFLDDGHGNCLLDVPRKQILGPEELPGQTYDATQQCNLTFGPEYSVCPGMDVCARLWCAVVRQGQMVCLTKKLPAVEGTPCGKGRICLQGKCVDKTKKKYYSTSSHGNWGSWGPWGQCSRSCGGGVQFAYRHCNNPAPRNSGRYCTGKRAIYRSCSVIPCPHHHHHHHHHH

SEQ ID NO: 152 - protein, 632 aa, Cavia porcellus
MLLGWASLLLCAFRLPQAAASAAAAPAQDKAGQPRAAAAAPQPRRRQGEHAPLRVEPPGHPHALAPQRRGRGLLQSIDRLYSGGGKVGYLVYAGGRRFLLDLERDGSVGAAGLFPAGGGLSAPRRHRSHCFYRGTVDGSPRSLAVFDLCGGLRGFFAVKHARYTVKPLLRGPWAEADTPRVYGDESARIPHVYTREGFSFEALPPRASCETPASQPGPHERPPAHNSPGRHSTVDPQLPELSALSPAGDPGQQIWWRRRRRSISRARQVELLLVADGSMAKMYGRGLQHYLLTLASIANRLYSHASIENHIRLAVVKVVVLGDKDKSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEENFGLTEDKRLMSSILTSIDASKPWSKCTSATMTEFLDDGHGNCLLDVPRKQIPSPEELPGQTYDATQQCNLTFGPEYSVCPGMDVCARLWCAVVRQGQMVCLTKKLPAVEGTPCGKGRICLQGKCVDKTKKKYYSTSSHGNWGSWGPWGQCSRSCGGGVQFAYRHCNNPAPRNSGRYCTGKRAIYRSCSVTPCPHHHHHHHHHH

SEQ ID NO: 153 - protein, 632 aa, Mus musculus
MRLEWAPLLLLLLLLSASCLSLAADSPAAAPAQDKTRQPQAAAAAAEPDQPQGEETRERGHLQPLAGQRRSGGLVQNIDQLYSGGGKVGYLVYAGGRRFLLDLERDDTVGAAGSIVTAGGGLSASSGHRGHCFYRGTVDGSPRSLAVFDLCGGLDGFFAVKHARYTLKPLLRGSWAEYERIYGDGSSRILHVYNREGFSFEALPPRASCETPASPSGPQESPSVHSRSRRRSALAPQLLDHSAFSPSGNAGPQTWWRRRRRSISRARQVELLLVADSSMARMYGRGLQHYLLTLASIANRLYSHASIENHIRLAVVKVVVLTDKDTSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEENFGTTEDKRLMSSILTSIDASKPWSKCTSATITEFLDDGHGNCLLDLPRKQILGPEELPGQTYDATQQCNLTFGPEYSVCPGMDVCARLWCAVVRQGQMVCLTKKLPAVEGTPCGKGRVCLQGKCVDKTKKKYYSTSSHGNWGSWGPWGQCSRSCGGGVQFAYRHCNNPAPRNSGRYCTGKRAIYRSCSVTPCPHHHHHHHHHH

SEQ ID NO: 154 - protein, 632 aa, Macaca mulatta
MLLGWASLLLCAFRLPLAAAGPAAAPAQDKAGQPATAAAAAQPRRRQGEEVQERTEPPGHPHPLAQRRSSKGLVQNIDQLYSGGGKVGYLVYAGGRRFLLDLERDGSVGTAGFVPTEGGTSAPWRHRSHCFYRGTVDGSPRSLAVFDLCGGLDGFFAVKHARYTLKPLLRGPWAEEETRRVYGDGSARILHVYTREGFSFEALQPRASCETPASTPEPHERPPAHSNPGGRAALASQLLDQSAVSPAGGPGPQTWWRRRRRSISRARQVELLLVADASMARLYGRGLQHYLLTLASIANRLYSHASIENHIRLAVVKVVVLGDKDKSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEETFGSTEDKRLMSSILTSIDASKPWSKCTSATITEFLDDGHGNCLLDQPRKQILGPEELPGQTYDATQQCNLTFGPEYSVCPGMDVCARLWCAVVRQGQMVCLTKKLPAVEGTPCGKGRICLQGKCVDKTKKKYYSTSSHGNWGSWGSWGQCSRSCGGGVQFAYRHCNNPAPRNNGRYCTGKRAIYRSCGLMPCPHHHHHHHHHH

SEQ ID NO: 155 - protein, 2415 aa, Homo sapiens
MTTLLWVFVTLRVITAAVTVETSDHDNSLSVSIPQPSPLRVLLGTSLTIPCYFIDPMHPVTTAPSTAPLAPRIKWSRVSKEKEVVLLVATEGRVRVNSAYQDKVSLPNYPAIPSDATLEVQSLRSNDSGVYRCEVMHGIEDSEATLEVVVKGIVFHYRAISTRYTLDFDRAQRACLQNSAIIATPEQLQAAYEDGFHQCDAGWLADQTVRYPIHTPREGCYGDKDEFPGVRTYGIRDTNETYDVYCFAEEMEGEVFYATSPEKFTFQEAANECRRLGARLATTGHVYLAWQAGMDMCSAGWLADRSVRYPISKARPNCGGNLLGVRTVYVHANQTGYPDPSSRYDAICYTGEDFVDIPENFFGVGGEEDITVQTVTWPDMELPLPRNITEGEARGSVILTVKPIFEVSPSPLEPEEPFTFAPEIGATAFAEVENETGEATRPWGFPTPGLGPATAFTSEDLVVQVTAVPGQPHLPGGVVFHYRPGPTRYSLTFEEAQQACPGTGAVIASPEQLQAAYEAGYEQCDAGWLRDQTVRYPIVSPRTPCVGDKDSSPGVRTYGVRPSTETYDVYCFVDRLEGEVFFATRLEQFTFQEALEFCESHNATATTGQLYAAWSRGLDKCYAGWLADGSLRYPIVTPRPACGGDKPGVRTVYLYPNQTGLPDPLSRHHAFCFRGISAVPSPGEEEGGTPTSPSGVEEWIVTQVVPGVAAVPVEEETTAVPSGETTAILEFTTEPENQTEWEPAYTPVGTSPLPGILPTWPPTGAETEESTEGPSATEVPSASEEPSPSEVPFPSEEPSPSEEPFPSVRPFPSVELFPSEEPFPSKEPSPSEEPSASEEPYTPSPPEPSWTELPSSGEESGAPDVSGDFTGSGDVSGHLDFSGQLSGDRASGLPSGDLDSSGLTSTVGSGLTVESGLPSGDEERIEWPSTPTVGELPSGAEILEGSASGVGDLSGLPSGEVLETSASGVGDLSGLPSGEVLETTAPGVEDISGLPSGEVLETTAPGVEDISGLPSGEVLETTAPGVEDISGLPSGEVLETTAPGVEDISGLPSGEVLETTAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETAAPGVEDISGLPSGEVLETTAPGVEEISGLPSGEVLETTAPGVDEISGLPSGEVLETTAPGVEEISGLPSGEVLETSTSAVGDLSGLPSGGEVLEISVSGVEDISGLPSGEVVETSASGIEDVSELPSGEGLETSASGVEDLSRLPSGEEVLEISASGFGDLSGVPSGGEGLETSASEVGTDLSGLPSGREGLETSASGAEDLSGLPSGKEDLVGSASGDLDLGKLPSGTLGSGQAPETSGLPSGFSGEYSGVDLGSGPPSGLPDFSGLPSGFPTVSLVDSTLVEVVTASTASELEGRGTIGISGAGEISGLPSSELDISGRASGLPSGTELSGQASGSPDVSGEIPGLFGVSGQPSGFPDTSGETSGVTELSGLSSGQPGVSGEASGVLYGTSQPFGITDLSGETSGVPDLSGQPSGLPGFSGATSGVPDLVSGTTSGSGESSGITFVDTSLVEVAPTTFKEEEGLGSVELSGLPSGEADLSGKSGMVDVSGQFSGTVDSSGFTSQTPEFSGLPSGIAEVSGESSRAEIGSSLPSGAYYGSGTPSSFPTVSLVDRTLVESVTQAPTAQEAGEGPSGILELSGAHSGAPDMSGEHSGFLDLSGLQSGLIEPSGEPPGTPYFSGDFASTTNVSGESSVAMGTSGEASGLPEVTLITSEFVEGVTEPTISQELGQRPPVTHTPQLFESSGKVSTAGDISGATPVLPGSGVEVSSVPESSSETSAYPEAGFGASAAPEASREDSGSPDLSETTSAFHEANLERSSGLGVSGSTLTFQEGEASAAPEVSGESTTTSDVGTEAPGLPSATPTASGDRTEISGDLSGHTSQLGVVISTSIPESEWTQQTQRPAETHLEIESSSLLYSGEETHTVETATSPTDASIPASPEWKRESESTAAAPARSCAEEPCGAGTCKETEGHVICLCPPGYTGEHCNIDQEVCEEGWNKYQGHCYRHFPDRETWVDAERRCREQQSHLSSIVTPEEQEFVNNNAQDYQWIGLNDRTIEGDFRWSDGHPMQFENWRPNQPDNFFAAGEDCVVMIWHEKGEWNDVPCNYHLPFTCKKGTVACGEPPVVEHARTFGQKKDRYEINSLVRYQCTEGFVQRHMPTIRCQPSGHWEEPRITCTDATTYKRRLQKRSSRHPRRSRPSTAH

SEQ ID NO: 156 - protein, 122 aa, Artificial Sequence
  Description of Artificial Sequence Synthetic polypeptide
EVQLVESGGGVVQPGGSLRLSCAASGSTFIINVVRWYRRAPGKQRELVATISSGGNANYVDSVRGRFTISRDNSKNTVYLQMNSLRPEDTALYYCNVPTTHYGGVYYGPYWGQGTLVTVSSA

SEQ ID NO: 157 - protein, 125 aa, Artificial Sequence
  Description of Artificial Sequence Synthetic polypeptide
EVQLVESGGGVVQPGGSLRLSCAASGRTFSSYTMGWFRQAPGKEREFVAAISWSGGRTYYADSVKGRFTISRDNSKNTVYLQMNSLRPEDTALYYCAAYRRRRASSNRGLWDYWGQGTLVTVSSA

SEQ ID NO: 158 - protein, 3 aa, Artificial Sequence
  Description of Artificial Sequence Synthetic peptide
AAA

SEQ ID NO: 159 - protein, 5 aa, Artificial Sequence
  Description of Artificial Sequence Synthetic peptide
GGGGS

SEQ ID NO: 160 - protein, 7 aa, Artificial Sequence
  Description of Artificial Sequence Synthetic peptide
SGGSGGS

SEQ ID NO: 161 - protein, 8 aa, Artificial Sequence
  Description of Artificial Sequence Synthetic peptide
GGGGGGGS

SEQ ID NO: 162 - protein, 9 aa, Artificial Sequence
  Description of Artificial Sequence Synthetic peptide
GGGGSGGGS

SEQ ID NO: 163 - protein, 10 aa, Artificial Sequence
  Description of Artificial Sequence Synthetic peptide
GGGGSGGGGS

SEQ ID NO: 164 - protein, 15 aa, Artificial Sequence
  Description of Artificial Sequence Synthetic peptide
GGGGSGGGGSGGGGS

SEQ ID NO: 165 - protein, 18 aa, Artificial Sequence
  Description of Artificial Sequence Synthetic peptide
GGGGSGGGGSGGGGGGGS

SEQ ID NO: 166 - protein, 20 aa, Artificial Sequence
  Description of Artificial Sequence Synthetic peptide
GGGGSGGGGSGGGGSGGGGS

SEQ ID NO: 167 - protein, 25 aa, Artificial Sequence
  Description of Artificial Sequence Synthetic peptide
GGGGSGGGGSGGGGSGGGGSGGGGS

SEQ ID NO: 168 - protein, 30 aa, Artificial Sequence
  Description of Artificial Sequence Synthetic polypeptide
GGGGSGGGGSGGGGSGGGGSGGGGSGGGGS

SEQ ID NO: 169 - protein, 35 aa, Artificial Sequence
  Description of Artificial Sequence Synthetic polypeptide
GGGGSGGGGSGGGGSGGGGSGGGGSGGGGSGGGGS

SEQ ID NO: 170 - protein, 40 aa, Artificial Sequence
  Description of Artificial Sequence Synthetic polypeptide
GGGGSGGGGSGGGGSGGGGSGGGGSGGGGSGGGGSGGGGS

SEQ ID NO: 171 - protein, 15 aa, Artificial Sequence
  Description of Artificial Sequence Synthetic peptide
EPKSCDKTHTCPPCP

SEQ ID NO: 172 - protein, 24 aa, Artificial Sequence
  Description of Artificial Sequence Synthetic peptide
GGGGSGGGSEPKSCDKTHTCPPCP

SEQ ID NO: 173 - protein, 12 aa, Artificial Sequence
  Description of Artificial Sequence Synthetic peptide
EPKTPKPQPAAA

SEQ ID NO: 174 - protein, 62 aa, Artificial Sequence
  Description of Artificial Sequence Synthetic polypeptide
ELKTPLGDTTHTCPRCPEPKSCDTPPPCPRCPEPKSCDTPPPCPRCPEPKSCDTPPPCPRCP

SEQ ID NO: 175 - protein, 4 aa, Artificial Sequence
  Description of Artificial Sequence Synthetic peptide
ARGS